(19) United States
(12) Patent Application Publication
(10) Pub. No.: US 2023/0330328 A1
(43) Pub. Date: Oct. 19, 2023

(54) SYSTEMS AND DEVICES FOR CONTROLLED DRUG DELIVERY

(71) Applicant: Bexson Biomedical, Inc., Santa Barbara, CA (US)

(72) Inventors: Sheldon Blain MOBERG, Santa Barbara, CA (US); Moshe BARKAN, Santa Barbara, CA (US); Jeffrey BECKER, Santa Barbara, CA (US); Gregg PETERSON, Santa Barbara, CA (US)

(21) Appl. No.: 18/009,612

(22) PCT Filed: Jun. 11, 2021
(86) PCT No.: PCT/US2021/037114

§ 371 Date: Dec. 9, 2022

Related U.S. Application Data
(60) Provisional application No. 63/081,085, filed on Sep. 21, 2020.
(60) Provisional application No. 63/038,618, filed on Jun. 12, 2020.

(51) Int. Cl. A61M 5/142; A61M 5/14; A61M 5/00

(52) U.S. Cl. CPC A61M 5/14248; A61M 5/1413; A61M 5/002; A61M 5/3213

(57) ABSTRACT

Provided herein are systems, devices, kits, and methods for administering liquid drug formulation by titratable subcutaneous or intramuscular administration.

### CROSS REFERENCE

This application claims the benefit of U.S. Provisional Application No. 63/038,618 filed Jun. 12, 2020 and U.S. Provisional Application No. 63/081,085 filed Sep. 21, 2020, each of which is incorporated herein by reference in its entirety.

### BACKGROUND

There are a multitude of medicinal therapies available today to treat diseases, conditions, or disorders that come in a range of modalities, including tablets, aerosols, powders, or liquids. In each of the available modalities, there is a vast range in the cost of therapy and the patient risk associated with the treatment. Additionally, some of the treatments include controlled substances that can be abused and lethal if not taken as prescribed by the intended patient.

### SUMMARY

For therapies that involve patient administration of a high value medicine, a controlled substance (e.g., ketamine), a substance with a high propensity for abuse or addiction, or a substance with harmful side effects if taken at an improper dose, it is important to ensure that only the prescribed patient has access to the medication and administers it as prescribed. In some cases, there are benefits of a liquid formulation that is designed for injection, such as by intramuscular or subcutaneous administration. Any high value or controlled substance in liquid formulation intended for intramuscular or subcutaneous administration and is intended for patient administration, or administered outside of the healthcare system controls (in-home), needs to ensure that only the prescribed patient received the therapy as indicated, including any suitable Schedule 1, Schedule 2, Schedule 3, or Schedule 4 drug as listed by the Controlled Substances Act. For instance, ketamine, an NMDA receptor antagonist that has found use in treating pain, depression, and numerous other psychiatric and physical disorders, can be a very effective drug when administered according to the recommendations of a medical professional but has a high potential for abuse. As another example, opioids such as oxycodone, hydrocodone, and fentanyl have a high potential for abuse that can lead to addiction and/or overdose.

When medicinal delivery is not under the direct control of a licensed health practitioner, there is a risk that the patient or intended user fails to follow the prescribed instructions for use that help ensure treatment is both safe and effective. For patients administered intramuscular or subcutaneous combination products outside of a healthcare facility, there may be a need or benefit to leverage the administration technology (device) to reduce the risk associated with the intended patient misusing the prescribed medicine. For example, a patient who receives the prescription might attempt to withdrawal the medication from the medicinal container or delivery device with the intent to deliver not as prescribed, provide it to someone other than the intended patient, or sell it. Accordingly, there exists a need for controlled drug delivery devices that a subject can administer at home that enforce strict requirement to treatment protocols provided by medical professionals.

In addition, some medications are designed for intramuscular or subcutaneous injections over time. This is the case with the liquid formulation of ketamine and numerous other drugs. Depending on the disease treated (e.g., depression, suicidal ideation, chronic pain, acute pain), various drugs can be formulated for subcutaneous basal delivery over several minutes, several hours or up to multiple days. In addition, multiple predetermined bolus deliveries can be delivered by patient action during the therapy period. Since many drugs, including ketamine, often require a titrated delivery over an extended period of time, a wearable pump or tubing-set based ambulatory infusion pump has the capability to deliver this medicine over the desired period of time. A benefit of the wearable pump is in the simplicity, and opportunity to design in tamper resistance to make it difficult to extract the medicine from the prescribed intended use. A patient use delivery system that is easy to operate and provides tamper resistance to the medication offers many advantages.

The systems, devices, kits, formulations, and methods provided herein provide an innovative solution to problems of abuse of prescribed drug substances formulated for delivery by injection, including subcutaneous and intramuscular administration. The systems, devices, kits, formulations, and methods also enable users to abide by dosing regimens prescribed by medical professionals, thus ensuring proper treatment and minimization of the risk of abuse or intentional or unintentional misuse of controlled substances prescribed by medical professionals.

One advantage of the systems, devices, kits, and methods provided herein compared to other systems for subcutaneous or intramuscular delivery by subjects at home is the prevention of a subject from being able to abuse the medication by attaching multiple devices comprising the drug formulation to his or her body at once. The drug formulation may be provided in one component of a drug delivery system or device that is detachable or separate from another component that controls delivery. This can help prevent multiple components containing the drug formulation from being administered simultaneously. For example, a subject may be provided with a component for controlling delivery and multiple cartridges or tamper resistant single-use drug formulation-containing reservoir components, wherein the component that controls delivery can only facilitate administration of the drug formulation from a single cartridge or tamper resistant single-use drug formulation containing reservoir at a time. A subject would be provided with only a single component for controlling delivery, for example, a user interface component comprising one or more buttons or other interactive elements for controlling administration or delivery of the drug formulation. This provides a limiting factor on the subject's ability to self-administer more than the necessary or prescribed amount of the drug formulation. By providing a drug delivery device system comprising a distinct drug formulation-containing reservoir component, and a separate user interface component, a medical professional can prescribe a kit for administering a dosage regimen spanning multiple reservoirs that cannot be taken simultaneously by the patient while still offering the tamper-proof protection of single component, fully integrated devices.

Another advantage of the systems, devices, kits, and methods provided herein are cost savings from decoupling the user interface component and the reservoir and other components of the device. As provided herein, the user interface and its electronic components can be reused with multiple disposable drug reservoir cartridges. This allows the cost of the reusable user interface to be amortized over multiple disposable components.

The systems, devices, kits, and methods provided herein also provide the advantage of improved manufacturability, particularly in the sterilization process of preparing pre-loaded, pre-filled drug delivery devices to patients. Single component, fully integrated drug delivery devices and drug delivery devices comprising a reusable user interface and single disposable component containing a drug reservoir pose manufacturing difficulties and complexities. This is due to the fact that drug containing reservoirs containing liquid drug formulations possess different sterilization requirements than other portions of the device, such as the flow path or other hard surfaces of the device. In certain embodiments, this difficulty is overcome by providing the reservoir portion as a separate component from the flow path and other hard surfaces, thus allowing two separate sterilization procedures to be employed, thus giving additional manufacturing control over the process.

Further, the systems, devices, kits, and methods provided herein couple the manufacturing and sterilization advantages described above with the tamper-proofing advantages of other pre-filled and pre-loaded drug delivery devices. This is accomplished by providing a component comprising a distinct drug reservoir inside of a tamper-proof container configured to dispense from single drug reservoir compartments according to a pre-determined dosage regimen. This allows a system or kit comprising two or more separate disposable components (e.g., a component comprising the drug formulation reservoir and/or a component comprising the flow path and other hard surface components) to still prevent a subject from abusing the drug by preventing the subject from taking multiple dosages at one time.

The systems, devices, kits, and methods provided herein also provide the advantage of ensuring that any air trapped in the delivery system can be removed prior to administration by the patient or subject, thus ensuring that a drug formulation requiring carefully titrated delivery is administered accurately and according to the proper dosage regimen. The innovative solution involves the placement of one or more sensors on the device whose sensor readings allow a determination or indication of orientation of the drug filled reservoir and/or the outlet through which the drug is administered. The one or more sensors can include a positional sensor and may be positioned on the interior of the device. In some cases, the positional sensor is coupled to a level indicator which may be positioned on an exterior surface of the device or on a component separate from the device, such as the subject's mobile phone. The level indicator informs the user when the reservoir is in the proper orientation to expel trapped air from the device and/or drug reservoir in the device or cartridge, thereby enabling removal of trapped air and allowing controlled and accurate dosing of the drug formulation.

In one aspect, provided herein, is a drug delivery system comprising a delivery device comprising: a) a pump or injection mechanism configured for administering a drug formulation from a reservoir; and b) an activation mechanism configured to selectively lock the pump or injection mechanism to prevent administration of the drug formulation. In some embodiments, the injection mechanism comprises a needle configured to administer the drug formulation. In some embodiments, the activation mechanism is configured to allow setting of a dosage of the drug formulation. In some embodiments, the delivery device is a single part prefilled injector. In some embodiments, the delivery device is a two part prefilled injector. In some embodiments, the delivery device is a three part injector. In some embodiments, the delivery device comprises a reusable injector component comprising an electronic control module. In some embodiments, the delivery device comprises a disposable component comprising the reservoir containing the drug formulation. In some embodiments, the delivery device comprises a magnetic or mechanical coupling mechanism for combining a reusable component and a disposable component making up the delivery device. In some embodiments, the delivery device comprises a shield activated trigger for unlocking the delivery device for administration of the drug formulation. In some embodiments, further comprising a tamper resistant package comprising one or more cartridges containing the reservoir. In some embodiments, the tamper resistant package comprises a plurality of cartridges. In some embodiments, the drug delivery system is configured to cause the tamper resistant package to release a cartridge from the plurality of cartridges upon obtaining user authorization. In some embodiments, the plurality of cartridges are individually locked prior to obtaining user authorization. In some embodiments, further comprising a cap assembly. In some embodiments, the cap assembly comprises a decontamination sponge. In some embodiments, further comprising a controlled cartridge septum lockout function. In some embodiments, the controlled cartridge septum lockout function is configured to open or close an iris to control access to the reservoir.

In another aspect, provided herein, is a drug delivery system comprising: a reservoir comprising a drug formulation; a drive mechanism configured to pump the drug formulation from the reservoir through a delivery needle upon activation; and a lockout mechanism configured to prevent unauthorized activation of the drive system. In some embodiments, the drug delivery system has a multi-component configuration comprising: a reusable component; and a disposable component housing the reservoir comprising the drug formulation. In some embodiments, the lockout mechanism keeps the drive mechanism locked until the reusable component and the disposable component are coupled. In some embodiments, the lockout mechanism comprises a magnet and a magnetic detection element that are brought into proximity upon coupling of the reusable component and the disposable component. In some embodiments, the magnet is an electromagnet housed within the reusable component and the magnetic detection element is housed within the disposable component. In some embodiments, coupling of the reusable component and the disposable component causes an electromagnetic field produced by the electromagnet to act upon the magnetic detection element, thereby unlocking the drive mechanism for authorized activation to administer the drug formulation. In some embodiments, the lockout mechanism is configured to provide active electronic control or passive mechanical control over activation of the drive mechanism. In some embodiments, the lockout mechanism is configured to unlock the drive mechanism using RFID, Near Field Communication (NFC), or radio frequency. In some embodiments, the drug delivery system comprises an electronic module configured to provide active control over activation of the drive mechanism. In some embodiments, the electronic module is configured to control the drive mechanism based on receipt of a signal indicating authorized activation. In some embodiments, the signal indicating authorized activation is provided by a wireless signal from a computing device. In some embodiments, the computing device is a desktop computer, a laptop computer, a tablet, or a smartphone. In some embodiments, the drug delivery system comprises a wearable pump. In some embodiments, the lockout mechanism is a completely mechanical mechanism providing control over activation of the drive mechanism without an electronic module.

In another aspect, provided herein, is a drug delivery system comprising: a reservoir comprising a drug formulation; a drive mechanism configured to pump the drug formulation from the reservoir through a delivery needle; and a needle protection system that blocks external access to the delivery needle until activation of a needle insertion mechanism. In some embodiments, activation of the needle insertion mechanism drives the delivery needle forward into an unretracted configuration and displaces a needle protection door that blocks external access to the delivery needle. In some embodiments, the delivery needle is in a retracted configuration when the needle insertion mechanism is inactivated. In some embodiments, the needle protection door blocks a delivery port through which the delivery needle extends in the unretracted configuration. In some embodiments, a fluid path connects the reservoir to the delivery needle. In some embodiments, the drug delivery system is a single component or a multi-component drug delivery device. In some embodiments, the drug delivery system comprises a wearable pump.

In another aspect, provided herein, is a drug delivery system comprising a watertight enclosure comprising: a reservoir comprising a drug formulation; a drive mechanism configured to pump the drug formulation from the reservoir through a delivery needle; and a water barrier system configured to selectively allow passage of gases while preventing passage of a liquid. In some embodiments, the water barrier system is configured to allow pressure equalization between the interior of the enclosure and the exterior of the enclosure. In some embodiments, the water barrier system comprises a water barrier membrane. In some embodiments, the water barrier membrane comprises a hydrophobic material. In some embodiments, the hydrophobic material comprises a hydrophobic polymer comprising polytetrafluoroethylene, polypropylene, polyvinylidene difluoride, or an acrylic polymer. In some embodiments, the water barrier membrane is positioned over a delivery port through which the delivery needle extends for delivery of the drug formulation. In some embodiments, the water barrier membrane is configured to maintain a watertight seal upon penetration by the delivery needle. In some embodiments, the water barrier membrane is constructed from a composite of different materials. In some embodiments, the water barrier membrane comprises a hydrophobic membrane and a needle sealing barrier configured to maintain the watertight seal upon penetration by the delivery needle. In some embodiments, the needle sealing barrier comprises silicone, low durometer polyethylene, butyl rubber, or high density foam. In some embodiments, the water barrier membrane is a single membrane or a multi-membrane. In some embodiments, the drug delivery system is a single component or a multi-component drug delivery device. In some embodiments, the drug delivery system comprises a wearable pump.

In another aspect, provided herein, is a drug delivery system comprising a watertight enclosure comprising: a reservoir comprising a drug formulation; a drive mechanism configured to pump the drug formulation from the reservoir; and a liquid absorbing material positioned in proximity to the reservoir. In some embodiments, the liquid absorbing material is configured to absorb a leak of the drug formulation from the reservoir. In some embodiments, the liquid absorbing material comprises an absorbent material formed from one or more of polyester, polyurethane, or vegetal cellulose. In some embodiments, the liquid absorbing material comprises an absorbent material that forms a solid or gelatin material upon contact with leaked liquid formulation. In some embodiments, the absorbent material comprises slush powder, wherein the slush powder optionally comprises sodium polyacrylate or silicon dioxide. In some embodiments, the reservoir holds the drug formulation within a breakable container. In some embodiments, the drug delivery system comprises a wearable pump.

In another aspect, disclosed herein is a drug delivery system comprising: a cartridge comprising a reservoir holding a drug formulation; a drive mechanism configured to pump the drug formulation from the reservoir; and an electronic module configured to detect damage to the reservoir. In some embodiments, drug delivery system comprises a conductive trace incorporated in or around the reservoir and coupled to the electronic module. In some embodiments, the electronic module is configured to detect damage to the cartridge based on a change in a property of the conductive trace. In some embodiments, the property of the conductive trace is impedance. In some embodiments, the electronic module is configured to lock the drive mechanism to prevent the drug formulation from being pumped from the reservoir upon detection of damage to the cartridge. In some embodiments, the conductive trace is wrapped around the outside surface of the cartridge. In some embodiments, the drug delivery system comprises a wearable pump.

In another aspect, provided herein, is a drug delivery system comprising: a cartridge comprising reservoir holding a drug formulation; a drive mechanism configured to pump the drug formulation from the reservoir when a fluid path is established between the reservoir and a cartridge needle; and a control module configured to selectively establish the fluid path between the reservoir and the cartridge needle by controlling extension of the cartridge needle into the cartridge. In some embodiments, the drug delivery system comprises a solenoid operatively coupled to the cartridge needle whereby retraction of the solenoid causes translation of the cartridge needle. In some embodiments, the retraction of the solenoid causes translation of the cartridge needle to penetrate a cartridge septum to establish the fluid path between the reservoir and the cartridge needle. In some embodiments, the control module comprises an electronic module or a mechanical module for controlling extension of the cartridge needle into the cartridge. In some embodiments, the electronic module is configured to establish the fluid path between the reservoir and the cartridge needle upon authorized activation of the drug delivery system. In some embodiments, the electronic module is configured to terminate the fluid path between the reservoir and the cartridge needle based on determination of one or more of: end of deliverable medicine within the primary container, end of intended therapy, error state, or premature removal of the delivery device from the body. In some embodiments, the mechanical module comprises a mechanical drive system configured to automatically extend the cartridge needle into the cartridge upon authorized activation of the drug delivery system and retract the cartridge needle from the cartridge upon completion of delivery of the drug formulation. In some embodiments, the drug delivery system comprises a wearable pump.

In another aspect, provided herein, is a pen injector comprising: an injector body comprising: a reservoir comprising a drug formulation; an injection mechanism configured to pump the drug formulation from the reservoir through a delivery needle; and a lockout mechanism configured to prevent unauthorized activation of the injection mechanism. In some embodiments, further comprising an injector cap configured to be removably coupled to the injector body, wherein the injector cap covers the delivery needle when coupled to the pen injector body. In some embodiments, the lockout mechanism is configured to lock the injector cap by preventing the injector cap from being decoupled or removed from the injector body without receiving an authorized activation. In some embodiments, the lockout mechanism is controlled by an electronic module configured to detect a signal providing the authorized activation from an external computing device. In some embodiments, the lockout mechanism is configured to automatically relock the injector cap following removal and recoupling to the injector body after use of the pen injector to administer the drug formulation. In some embodiments, the lockout mechanism is a mechanical or an electronic mechanism. In some embodiments, further comprising a fingerprint scanner wherein detection of an authorized user by the fingerprint scanner causes the lockout mechanism to unlock the injection mechanism. In some embodiments, the lockout mechanism unlocks the injection mechanism based upon receipt of a signal from a mobile device indicating detection of the authorized user using the fingerprint scanner. In some embodiments, the fingerprint scanner is located on the mobile device. In some embodiments, the pen injector is configured to deliver a fixed delivery dose. In some embodiments, the pen injector is configured to deliver one or more doses according to a factory set unchangeable dosage regimen. In some embodiments, the pen injector is configured to deliver one or more doses according to a programmable dosage regimen. In some embodiments, the programmable dosage regimen is set using an authorized programming device. In some embodiments, the authorized programming device is a computing device of a healthcare provider for a user of the pen injector or a mobile device of the user. In some embodiments, the lockout mechanism comprises a septum lockout mechanism to prevent unauthorized access to the reservoir. In some embodiments, the septum lockout mechanism comprises an iris that opens to provide authorized access to the reservoir. In some embodiments, the iris is configured to be opened upon rotation of an iris activation body.

In another aspect, disclosed herein, is a pen injector comprising: a reservoir comprising a drug formulation; an injection mechanism configured to pump the drug formulation from the reservoir through a delivery needle; and an adjustable dose feature configured to allow user dose selection; and an injector cap configured to be removably coupled to the injector body.

In another aspect, disclosed herein, is a pen injector comprising: an injector body comprising: a reservoir comprising a drug formulation; and an injection mechanism configured to pump the drug formulation from the reservoir through a delivery needle; and a needle assembly comprising a decontamination element for decontaminating an interface between the custom needle assembly and the injector body. In some embodiments, the decontamination element comprises a decontamination sponge containing an antimicrobial compound. In some embodiments, the reservoir comprises a septum whereby the decontamination element automatically comes into contact with or wipes the septum when the needle assembly is coupled to the injector body.

In another aspect, provided herein, is a pen injector comprising: a reservoir comprising a drug formulation; and an injection mechanism configured to pump the drug formulation from the reservoir through a delivery needle; and a needle shield feature configured to inhibit activation of the injection mechanism. In some embodiments, the needle shield feature is configured to inhibit activation of the injection mechanism until the needle shield feature is fully depressed by pressure from the pen injector being pressed against an injection site. In some embodiments, the needle shield feature is configured to allow automatic injection by the injection mechanism when the needle shield feature is fully depressed by pressure from the pen injector being pressed against an injection site. In some embodiments, the needle shield feature is configured to unlock the pen injector when the pen injector is pressed against an injection site, thereby allowing user control of injection by pressing an injection or activation button. In some embodiments, the needle shield feature comprises an alcohol wipe for disinfecting an injection site.

In some aspects, provided herein, is a drug delivery device comprising a) a pump mechanism configured for administering a drug formulation from a reservoir; and b) a user interface comprising an indicator configured to display orientation information of an outlet of the reservoir, wherein the pump mechanism is configured to expel air from the reservoir when the indicator displays that the outlet is oriented in an upward direction.

In some embodiments, the outlet of the reservoir is not visible from a position exterior of the device.

In some embodiments, the pump mechanism is configured to automatically expel the air from the reservoir when the indicator displays that the outlet is oriented in the upward direction. In some embodiments, the user interface prompts a user to manually operate the pump to expel the air from the reservoir when the outlet is oriented in an upward direction. In some embodiments, the pump mechanism is configured to stop the pump once all of the air is expelled from the reservoir. In some embodiments, the pump mechanism comprises a torque or force sensor configured to automatically stop the pump mechanism when an increase in pressure is detected. In some embodiments, the pump mechanism comprises a torque or force sensor configured to stop the pump mechanism in response to an input from a user.

In some embodiments, expelling air from the reservoir comprises engaging the pump mechanism. In some embodiments, expelling the air from the reservoir comprises driving the air through an injection needle. In some embodiments, expelling the air from the reservoir comprises driving through a membrane, wherein the membrane is permeable to air and impermeable to fluids. In some embodiments, the membrane comprises a sensor configured to detect when fluid contacts the membrane and stop the pump mechanism.

In some embodiments, comprising an accelerometer and/or positional sensor configured to detect the orientation information of the outlet. In some embodiments, the accelerometer and/or positional sensor comprises a triaxial accelerometer, a triaxial gyroscope, a triaxial geomagnetic sensor, or any combination thereof. In some embodiments, the accelerometer and/or positional sensor comprise a microchip integrated into an electronic system of the drug delivery device.

In some embodiments, the indicator comprises a light or graphical display. In some embodiments, the indicator comprises a multi-colored light system, wherein different colors of light indicate different orientation statuses. In some embodiments, the indicator comprises a multiple segment light-bar configured to convey orientation information. In some embodiments, the indicator comprises a graphical display. In some embodiments, the graphical display further displays instructions for the user. In some embodiments, the user interface is attached to the device. In some embodiments, the user interface is a wireless enabled device in wireless communication with the drug delivery device. In some embodiments, the user interface further comprises additional information about the drug delivery device and/or the drug formulation. In some embodiments, the user interface allows a subject to self-administer the dose of the drug formulation.

In some embodiments, the drug delivery device is a single component fully integrated device. In some embodiments, the drug delivery device is a multi-component device assembled by a user. In some embodiments, the drug delivery device is configured to attach to a subject. In some embodiments, the drug delivery device is configured for titrated delivery. In some embodiments, the drug delivery device is configured to deliver the drug formulation over a pre-determined period of time. In some embodiments, the drug delivery device is configured for intramuscular or subcutaneous administration of the drug formulation. In some embodiments, the drug delivery device is pre-filled and/or pre-loaded.

In another aspect, provided herein, is a drug delivery device comprising: a) a user interface component comprising a user interface allowing a subject to self-administer a dose of a drug formulation; b) a reservoir component comprising a reservoir comprising the drug formulation; c) a pump mechanism configured for administering the drug formulation; and d) a system for expelling air from the drug delivery device; wherein the user interface and the reservoir are distinct components configured to be assembled by the subject; and wherein the user interface is configured to administer a pre-programmed dosage regimen, the pre-programmed dosage regimen requiring multiple reservoir components to be used sequentially.

In some embodiments, the reservoir component is configured to not administer the drug formulation in the absence of the user interface component. In some embodiments, the reservoir component further comprises the pump mechanism or a portion thereof, the system for expelling air or a portion thereof, a fluid path configured to deliver the dose of the drug formulation, or a component for attaching the device to the subject, or any combination thereof.

In some embodiments, the user interface component further comprises electronics, a power system, the pump mechanism or a portion thereof, the system for expelling air or a portion thereof, or a component for attaching the device to the subject, or any combination thereof. In some embodiments, the user interface component is reusable and the reservoir component is disposable.

In some embodiments, the reservoir component comprises an identification tag configured to be read by the user interface component, wherein the identification tag contains information about the pre-programmed dose regimen and/or the drug formulation.

In some embodiments, the user interface component is in wireless communication with a wireless enabled device, the wireless enabled device configured to program the pre-programmed dosage regimen.

In another aspect, provided herein, is a drug delivery device comprising: a) a user interface component comprising a user interface allowing a subject to self-administer a dose of a drug formulation; b) a pump mechanism configured for administering the drug formulation; c) a reservoir component comprising a reservoir comprising the drug formulation; and d) a body contact surface component configured for attachment of the device to the subject's body; wherein the user interface, the reservoir, and the body contact surface are each distinct components configured to be assembled by the subject.

In some embodiments, the reservoir component and the body contact surface component are disposable. In some embodiments, the user interface component is reusable with a plurality of reservoir components and/or body contact surface components.

In some embodiments, the body contact surface component further comprises a fluid path configured to deliver the dose of the drug formulation, the pump mechanism or a portion thereof, a system for expelling air from the delivery device or a portion thereof, or any combination thereof.

In some embodiments, the reservoir component is configured to not administer the drug formulation in the absence of the user interface component. In some embodiments, the body contacting surface component is sterilized separately from the other components prior to assembly. In some embodiments, the body contacting surface component is stored in a sterilized blister pack prior to assembly.

In another aspect, provided herein, is a kit for assembling the drug delivery device comprising: a) a user interface component comprising a user interface allowing a subject to self-administer a dose of a drug formulation; b) a pump comprising a pump mechanism configured for administering the drug formulation; c) a plurality of reservoir components each comprising a reservoir comprising the drug formulation; and d) a plurality of body contact surface components each configured for attachment of the device to the subject; wherein the user interface, a single reservoir component, and a single body contact surface component are each distinct components configured to be assembled by the subject; and wherein the plurality of reservoir components are stored in a tamper resistant package configured to dispense a subset of the reservoir components according to a pre-programmed dosage regimen.

In some embodiments, the tamper resistant package is structurally sound to inhibit access other than according to the pre-programmed dosage regimen.

In some embodiments, the tamper resistant package comprises a locking mechanism configured to open and allow access to the subset of the reservoirs after a specified period of time in the pre-programmed dosage regimen. In some embodiments, the tamper resistant package dispenses the subset of the reservoirs in response to a signal from the user interface component, a user mobile device, or a pump component containing the pump mechanism.

In some embodiments, the kit further comprises a sterilizing agent for sterilizing or decontaminating an exterior surface of a reservoir of the plurality of reservoirs.

In another aspect, provided herein, is a drug delivery device comprising: a) a pump mechanism configured to administer a drug formulation from a reservoir; and b) a sensor configured to detect a signal corresponding to one or more of a position or acceleration of the drug delivery device or reservoir; c) a user interface configured to indicate whether the pump mechanism is primed to expel gas from the drug reservoir based on an orientation of the drug delivery device or reservoir determined using the signal of the sensor.

In another aspect, provided herein, is a tamper resistant dispenser comprising: a housing providing an internal storage space configured to hold one or more components for facilitating drug delivery, the housing comprising an access port for dispensing the one or more components; a locking mechanism configured to prevent the one or more components from being dispensed from the access port without an authorization signal; and an electronic module configured to unlock the locking mechanism based on receipt of the authorization signal. In some embodiments, the authorization signal is provided through a user interface of the tamper resistant dispenser or a mobile device. In some embodiments, the user interface or the mobile device provides the authorization signal based on a passcode provided by a user. In some embodiments, the one or more components comprises at least one cartridge comprising a reservoir holding a drug formulation, a pump comprising a pump mechanism, an autoinjector, a body contact surface component configured for attachment to a subject. In some embodiments, the dispenser is configured to dispense each of the one or more components individually upon receiving the authorization signal.

In another aspect, disclosed herein is a prefilled and preloaded pen injector comprising: a) a prefilled reservoir containing a drug formulation; b) an injector mechanism comprising a septum configured to receive an injector needle; c) an activation mechanism configured to, upon receiving user input, activate the injector mechanism to deliver a dose of the drug formulation from the prefilled reservoir to a patient through the injector needle; and d) a lockout mechanism configured to, when engaged, prevent the injector mechanism from delivering the dose of the drug formulation from being activated by the activation mechanism, wherein the lockout mechanism is configured to control timing of delivery of the dose of the drug formulation.

In some embodiments, the drug formulation comprises ketamine. In some embodiments, the drug formulation comprises a dissociative medication compound, a dissociative hallucinogen compound, a dissociative anesthetic compound, an arylcyclo-hexylamine, a 1,2-diarylethylamine, a β-keto-arylcyclohexylamine, or a compound that modulates the NMDA receptor. In some embodiments, the drug formulation comprises a pharmaceutical compound having psychedelic properties. In some embodiments, the drug formulation comprises an opioid. In some embodiments, the drug formulation comprises an empathogenic or entactogenic compound. In some embodiments, the drug formulation comprises a gamma-aminobutyric acid (GABA) receptor antagonist.

In some embodiments, the activation mechanism comprises an activation button that is configured to activate the injector mechanism upon being depressed by a user.

In some embodiments, the lockout mechanism is a mechanical lockout timing mechanism. In some embodiments, the mechanical lockout timing mechanism comprises a spring-driven mechanical timepiece. In some embodiments, the mechanical lockout timing mechanism is configured to limit a number of doses that can be delivered within a set time period.

In some embodiments, the lockout mechanism is an electronic lockout timing mechanism. In some embodiments, the electronic lockout timing mechanism comprises an Application Specific Integrated Circuit (ASIC) and a crystal configured to control timing of the dose. In some embodiments, the electronic lockout timing mechanism is configured to limit a number of doses that can be delivered within a set time period. In some embodiments, power to the electronic lockout timing mechanism is initiated upon receiving user input activating the activation mechanism. In some embodiments, the electronic lockout timing mechanism comprises an electromagnet configured to release a lever upon disengagement of the electronic lockout timing mechanism, thereby unlocking the injector mechanism to allow delivery of the dose of the drug formulation.

In some embodiments, the pen injector further comprises a user interface comprising a readiness indicator showing a readiness status of the pen injector for delivery of the dose of the drug formulation. In some embodiments, the user interface comprises a biometric sensor for recognizing an authorized user, wherein the lockout mechanism is configured to be engaged unless at least the authorized user is recognized by the biometric sensor. In some embodiments, the readiness indicator comprises one or more LED lights.

In some embodiments, the user interface is configured to allow pairing of the pen injector with a mobile device. For example, the pen injector can have a processor and/or network element (e.g., transceiver microchip for WiFi or Bluetooth wireless communications) for communicating with the mobile device. In some embodiments, the mobile device is a smartphone, a tablet, or a laptop. In some embodiments, the pen injector is configured to receive authorization from the mobile device to disengage the lockout mechanism. In some embodiments, the pen injector is configured to communicate with the mobile device through a software application installed on the mobile device that provides the authorization.

In some embodiments, all components of the pen injector are integrated in a single-part design.

In some embodiments, all components of the pen injector are integrated in a two-part design. In some embodiments, the two-part design comprises a disposable pen injector body containing the prefilled reservoir and a reusable pen injector driver containing the injector mechanism, the activation mechanism, and the lockout mechanism. In some embodiments, the disposable pen injector body is configured to prevent access to the prefilled reservoir unless it is coupled to the reusable pen injector driver.

In some embodiments, the pen injector incorporates on-device unlocking elements, such as a fingerprint scanner, to identify the authorized patient and only unlock for that patient. In some embodiments, the pen injector is designed such that a fingerprint is necessary in order to unlock the pen for a single bolus delivery or pattern of bolus deliveries. In some cases, once the device has learned the authorized fingerprint, only an authenticated scan of this fingerprint will unlock the pen for any additional bolus injections. Instead of utilizing a fingerprint scanner, a combination lock, optical scan of the patient's face, or patient provided key can be utilized to unlock the pen injector for any purpose.

In another aspect, disclosed herein is a prefilled and preloaded pen injector comprising: a) a prefilled reservoir containing a drug formulation; b) an injector mechanism comprising a septum configured to receive an injector needle; c) an activation mechanism configured to, upon receiving user input, activate the injector mechanism to deliver a dose of the drug formulation from the prefilled reservoir to a patient through the injector needle; e) and a cartridge septum lockout feature to inhibit unintended access to the cartridge septum. Having access to the cartridge septum could allow for unauthorized or unintended withdrawal of medication by using an empty syringe to puncture the cartridge septum and withdrawal the medication from the prefilled cartridge within the pen injector. Accordingly, in some embodiments, a programmable and software controlled lockout cap is incorporated within the pen injector to inhibit access to the cartridge septum until authorized by time delay between uses, mobile device command, fingerprint scan, combination lock, key, or other methods of authorization or unlocking.

In some embodiments, the cartridge septum lockout utility is accomplished by implementing a septum lockout door or iris located in front of the cartridge septum, within the pen injector, to inhibit access to the cartridge septum. In the use of a pen injector, a single use sterile disposable needle assembly is attached to the pen injector in which the patient needle punctures the cartridge septum providing access to the medicine contained within the primary container such as a cartridge assembly. In some embodiments, the single use disposable pen needle assembly incorporates a decontamination solution, such as isopropyl alcohol, to clean the cartridge septum prior to the delivery needle penetration. In alternative embodiments, the pen injector cap incorporates a decontamination solution such as isopropyl alcohol to decontaminate the cartridge septum whenever the pen injector cap is placed on the pen injector.

In some embodiments, the pen injector incorporates some or all of the delivery lockout controls and some or all of the cartridge septum lockout utility described herein.

In another aspect, disclosed herein is a prefilled and preloaded pen injector comprising: a) a prefilled reservoir containing a drug formulation; b) an injector mechanism comprising a septum configured to receive an injector needle; c) an activation mechanism configured to, upon receiving user input, activate the injector mechanism to deliver a dose of the drug formulation from the prefilled reservoir to a patient through the injector needle; e) a secondary needle shield feature to inhibit device activation until fully depressed as a result of pen injector placement against the patient's body. The needle shield feature can perform the function of obscuring the needle insertion from the patient during administration.

In some embodiments, the prefilled and preloaded pen injector is directly activated for patient administration by the needle shield activation feature only when the lockouts controlling delivery are lifted. For example, if and when the pen injector lockout mechanism becomes disengaged via one or many of the mechanisms described in this document, the injector mechanism can be triggered to deliver the dose of the drug formulation simply by being pressed firmly into the skin to engage the needle shield activation mechanism.

In another aspect, disclosed herein is a prefilled and preloaded single bolus delivery pen injector comprising: a) a prefilled reservoir containing a drug formulation; b) an injector mechanism comprising a staked needle syringe; c) an activation mechanism configured to, upon receiving user input, activate the injector mechanism to deliver a dose of the drug formulation from the prefilled reservoir to a patient through the injector needle; and d) a lockout mechanism configured to, when engaged, prevent the injector mechanism from delivering the dose of the drug formulation from being activated by the activation mechanism, wherein the lockout mechanism is configured to control timing of delivery of the dose of the drug formulation.

In some embodiments, the single bolus delivery pen injector is referred to as an autoinjector that will deliver a single bolus following an authorized unlock input. The autoinjector could contain some or all of the delivery lockout features described herein. In some embodiments, the autoinjector will comprise a prefilled and preloaded cartridge primary container and one or more of the cartridge septum lockout features described herein. In some embodiments, the autoinjector will comprise a prefilled and preloaded staked needle syringe that will include needle lockout features with the same utility as defined herein.

Disclosed herein, in some aspects, is a drug delivery system comprising a delivery device comprising: a) a pump or injection mechanism configured for administering a drug formulation from a reservoir; and b) an activation mechanism configured to selectively lock the pump or injection mechanism to prevent administration of the drug formulation. In some embodiments, the injection mechanism comprises a needle configured to administer the drug formulation. In some embodiments, the activation mechanism is configured to allow setting of a dosage of the drug formulation. In some embodiments, the delivery device is a single part prefilled injector. In some embodiments, the delivery device is a two part prefilled injector. In some embodiments, the delivery device is a three part injector. In some embodiments, the delivery device comprises a reusable injector component comprising an electronic control module. In some embodiments, the delivery device comprises a disposable component comprising the reservoir containing the drug formulation. In some embodiments, the delivery device comprises a magnetic or mechanical coupling mechanism for combining a reusable component and a disposable component making up the delivery device. In some embodiments, the delivery device comprises a shield activated trigger for unlocking the delivery device for administration of the drug formulation. In some embodiments, the device further comprises a tamper resistant package comprising one or more cartridges containing the reservoir. In some embodiments, the tamper resistant package comprises a plurality of cartridges. In some embodiments, the drug delivery system is configured to cause the tamper resistant package to release a cartridge from the plurality of cartridges upon obtaining user authorization. In some embodiments, the plurality of cartridges are individually locked prior to obtaining user authorization. In some embodiments, the system further comprises a cap assembly. In some embodiments, the cap assembly comprises a decontamination sponge. In some embodiments, the system further comprises a controlled cartridge septum lockout function. In some embodiments, the controlled cartridge septum lockout function is configured to open or close an iris to control access to the reservoir.

The systems, devices, kits, and dispensers disclosed herein are contemplated to include any combination of the various features described within the body of this document. The single component and multi-component devices, whether they are a wearable pump/injection device or a pen injector, may each include one or more of the elements described herein. For example, the systems, devices, kits, and dispensers may include various elements or features such as a drive system lockout feature (e.g., locking mechanism), needle protection feature, water intrusion membrane, leaked fluid detection/conversion, broken container/cartridge detection/monitoring, control of cartridge septum fluid path connection, activation button for drug delivery, locking (and relocking) of the pen injector cap, predetermined factory set dosage regimen/delivery profile, programmable dosage regimen/delivery profile, fixed delivery mechanism, dial-a-dose mechanism, device unlocking features (e.g., fingerprint, mobile device), pen injector cap lockout feature, septum lockout feature, needle assembly for locking out septum access which can include alcohol wipe function, pen injector cap with decontamination feature (e.g., septum wipe), needle shield assembly, and various other features described throughout the present disclosure. Accordingly, embodiments described with respect to one aspect of a delivery device are also contemplated for other aspects.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 schematically illustrates a computer control system of a drug delivery device that is programmed or otherwise configured to implement methods provided herein.

FIG. 2 shows a non-limiting embodiment of a single-use prefilled and preloaded integrated disposable patch pump.

FIG. 3A shows a non-limiting embodiment of a two-part wearable delivery system with a reusable component and a disposable component.

FIG. 3B shows a non-limiting illustration of a magnetic drive coupling interface that can be utilized in a two-part delivery system such as a delivery pump.

FIG. 3C shows non-limiting embodiment of a side view of the two-part delivery system.

FIG. 3D shows non-limiting embodiment of an A-A section view of a two-part delivery system with a mechanical drive coupling interface.

FIG. 3E shows non-limiting embodiment of an A-A section view of a two-part delivery system with a magnetic drive coupling interface.

FIG. 4 shows a non-limiting embodiment of a two-part wearable delivery system with an RFID tag incorporated into the disposable component.

FIG. 5 schematically illustrates a non-limiting embodiment of a drug delivery device in wireless communication with a mobile phone or other wireless enabled device.

FIG. 6 shows a cross section view of a non-limiting embodiment of a pre-filled cartridge comprising an air bubble leftover from filling the device.

FIG. 7 shows a cross section view of a non-limiting embodiment of a pre-filled cartridge comprising an air bubble in the proper orientation for expelling the air from the device.

FIG. 8 shows a cross sectional view of a non-limiting embodiment of a pre-filled cartridge after an air bubble has been expelled from the device.

FIG. 9 shows a non-limiting embodiment of a device comprising a positional sensor and an indicator displaying orientation information placed on a user interface of a device.

FIG. 10A shows a cross-sectional view of a non-limiting embodiment of the disposable component of the two-part wearable delivery system in the locked state.

FIG. 10B shows a cross-sectional view of a non-limiting embodiment of the reusable component of the two-part wearable delivery system in the locked state.

FIG. 11 shows a close-up cross-sectional view of a non-limiting embodiment of the combined disposable and reusable two-part wearable delivery system displaying an unlocked delivery system.

FIG. 12 shows a non-limiting embodiment of the wearable delivery system with the needle port covered when the needle insertion mechanism is not activated, including a top view of the delivery system (FIG. 12A), a side cross-sectional view with the delivery port covered (FIG. 12B), a bottom view with the delivery port covered (FIG. 12C), and another side cross-sectional view showing the needle protection system (FIG. 12D) that is expanded in a close-up C-C view with the needle port covered (FIG. 12E).

FIG. 13 shows a non-limiting embodiment of the wearable delivery system with the needle port covered when the needle insertion mechanism is activated, including a side cross-sectional view showing the needle protection system (FIG. 13A) that is expanded in a close-up E-E view with the needle port covered (FIG. 13B).

FIG. 14 shows a non-limiting embodiment of a water barrier membrane with FIG. 14A showing a single-part water barrier membrane and FIG. 14B showing a composite water barrier membrane.

FIG. 15 shows a non-limiting embodiment of the cartridge or reservoir and electronic module of a wearable device with an isometric view (FIG. 15A) and a top cross-sectional view (FIG. 15B).

FIG. 16 shows a non-limiting embodiment of the cartridge or reservoir and electronic module of a wearable device with an isometric view (FIG. 16A) and a top cross-sectional view (FIG. 16B).

FIG. 17 shows a non-limiting embodiment of the cartridge or reservoir and electronic module of the wearable pump with a configuration where the cartridge needle is not fluidly coupled to the cartridge (FIG. 17A) and a configuration where the cartridge needle is fluidly coupled to the cartridge (FIG. 17B).

FIG. 18A, FIG. 18B, and FIG. 18C show a non-limiting embodiment of a single-part prefilled and preloaded (fixed bolus) pen injector.

FIG. 19A shows a section view of a non-limiting embodiment of the single-part prefilled and preloaded pen injector. FIG. 19B shows a close-up section view with the lock-out mechanism engaged to lock out the activation button. FIG. 19C shows a close-up section view with the lock-out mechanism disengaged. FIG. 19D shows an operational sequence for the single-part prefilled and preloaded pen injector.

FIG. 20A shows a close-up section view of the lockout mechanism engaged to lock out the cap for a non-limiting embodiment of the single-part prefilled and preloaded pen injector. FIG. 20B shows a close-up section view of the lockout mechanism disengaged.

FIG. 21 shows a non-limiting embodiment of a single-part prefilled and preloaded (fixed bolus dose) pen injector with a connectivity interface.

FIG. 22 shows a non-limiting embodiment of a single-part prefilled and preloaded pen injector with a patient adjustable dose feature and mobile authorization.

FIG. 23A shows a section view of a non-limiting embodiment of a single-part prefilled and preloaded pen injector with a lockout feature using a mechanical escapement allowing for one button activation. FIG. 23B shows a top-down view of the pen injector in a locked and unlocked configuration.

FIG. 24A and FIG. 24B show a non-limiting embodiment of a two-part prefilled and preloaded pen injector with a mechanical interface for coupling of the pen injector driver and the pen injector body.

FIG. 25A shows a non-limiting embodiment of a two-part prefilled and preloaded pen injector body with a mechanical interface for coupling of the pen injector driver and the pen injector body and an interface comprising a patient adjustable dose feature. FIG. 25B shows a close-up view of the dose set window and readiness LED indicator of a pen injector configured to communicate with a second device such as a mobile phone.

FIGS. 26A and 26B show a non-limiting embodiment of a two-part prefilled and preloaded patient adjustable (dial-a-dose) pen injector having an electronically controlled gearmotor drive system with the activation button and dial-a-dose knob.

FIG. 27 shows a non-limiting embodiment of fingerprint authorization for a single part prefilled and preloaded patient fixed dose pen injector or autoinjector

FIG. 28 shows a non-limiting embodiment of a mobile device being used to unlock an injector.

FIG. 29A, FIG. 29B, FIG. 29C, and FIG. 29D depict a non-limiting embodiment of a cartridge septum lockout feature to inhibit unintended access to the cartridge septum.

FIG. 30A, FIG. 30B, FIG. 30C, FIG. 30D, and FIG. 30E show a non-limiting embodiment of a single or two part prefilled and preloaded fixed or patient adjustable pen injector with a controlled cartridge septum lockout function using an iris and standard needle assay.

FIGS. 31A-31G shows a non-limiting embodiment of a custom patient needle assembly that incorporates a decontamination sponge for the purpose of decontaminating the cartridge septum when the custom needle assembly is placed onto the pen injector.

FIGS. 32A-32D shows a non-limiting embodiment of a pen cap assembly that incorporates the decontamination sponge with a pen cap iris to keep the decontamination sponge from drying out.

FIGS. 33A-33E shows a non-limiting embodiment of a pen injector or autoinjector that incorporates a shield activated trigger that unlocks the injection device enabling injection by the device button activation.

FIGS. 34A-34B shows a non-limiting embodiment of a tamper resistant package designed to release one or more prefilled cartridges after authorization.

FIG. 35 shows a non-limiting embodiment of a tamper resistant package designed to release a reservoir component after an authorizing signal is received by either the wearable delivery system or by a mobile phone.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

Disclosed herein are systems, devices, and methods for administering one or more doses of a drug formulation such as a ketamine formulation according to a controlled delivery mechanism that helps ensure accurate dosage. An air or gas aspiration mechanism and/or interface allows for efficient and effective removal of excess air or gas within the drug reservoir to help ensure appropriate titrated delivery. This is especially beneficial in the case of repeated titrated delivery of a drug in which excess air or gas during any of the deliveries can result in inaccurate dosing. The administration of the drug formulation can be provided according to one or more pre-programmed dosage regimen. The dose is often administered by subcutaneous or intravenous injection using a programmable delivery device. The delivery device can allow for treatment both in the clinic or hospital setting under supervision of a healthcare provider or via self-administration at home. The delivery device can be a pen injector or autoinjector. A doctor or healthcare provider is able to program the delivery device with one or more dosage regimen(s) and optionally sets dosage limits or other limits on the subject's ability to alter the dose and/or dosage regimen(s). The dosage regimen(s) can include selectable dosage options that give the subject limited control over the dose. The device is generally configured to be tamper resistant to prevent unauthorized access to the drug formulation stored on the device. Alternatively or in combination, the drug formulation is stored in a tamper resistant cartridge or vessel that is operably and/or detachably connected to the delivery device. In some cases, the device is remotely programmable to enable a doctor or healthcare provider to configure or modify the dosage regimen(s) via a network connection without requiring the subject to travel to the clinic or hospital. Self-administration of the drug formulation according to the pre-programmed dosage regimen(s) can allow an effective plasma concentration of the active ingredient to be reached and maintained outside of the clinic setting and without requiring large bolus infusions. Accordingly, plasma concentration fluctuation may be reduced compared to standard of care treatments at home.

In some embodiments, the systems, devices, kits, formulations, and methods disclosed herein help mitigate one or more side effect(s) of the main active ingredient and/or metabolites thereof. In some embodiments, the systems, devices, kits, formulations, and methods disclosed herein help mitigate the side effect(s) of ketamine or other controlled substance administration for treating physical, neurological and psychiatric disorder(s). For example, a side effect of ketamine includes hallucination, disorientation, dissociation, dizziness, drowsiness, increased heart rate, elevated blood pressure, nausea, vomiting, fatigue, brain fog, confusion, anxiety, distress, shortness of breath.

In some embodiments, the systems, devices, kits, formulations, and methods disclosed herein are used to administer a drug formulation comprising ketamine or other NMDA agonist, another controlled substance (e.g., an opioid), or other drug with a high risk of abuse, severe side effects, or create a high risk of dependency.

The administration of non-ketamine compounds using the systems, devices, kits, formulations, and methods disclosed herein is contemplated. In some embodiments, the drug formulation comprises a dissociative medication compound, a dissociative hallucinogen compound, a dissociative anesthetic compound, an arylcyclo-hexylamine, a 1,2-diarylethylamine, a β-keto-arylcyclohexylamine, or a compound that modulates the NMDA receptor. In some embodiments, the drug formulation comprises a pharmaceutical compound having psychedelic properties such as tryptamines, phenethylamines, and lysergamide classes of molecules. In some embodiments, the drug formulation comprises an opioid such as, for example, racemorphan, levorphanol, racemethorphan, buprenorphine, morphine, loperamide, morphine, codeine, hydrocodone, oxymorphone, buprenorphine, fentanyl, methadone, tramadol, alpha-methyl acetyl fentanyl, alfentanil, butyryl fentanyl, butyrfentanyl, carfentanil, 3-methylcarfentanil, 4-fluorofentanyl, beta-hydroxyfentanyl, alpha-methylfentanyl, cis-3-methylfentanyl, beta-hydroxy-3-methylfentanyl, remifentanil, sufentanil, 3-methylthiofentanyl, naloxone, or naltrexone. In some embodiments, the drug formulation comprises an empathogenic or entactogenic compound such as cathinones, 3,4-methylenedioxyampehtamines, aminoalkyl-substituted benzofurans, amphetamines, aminoindanes, stimulants, and other compounds. In some embodiments, the drug formulation comprises a gamma-aminobutyric acid (GABA) receptor antagonist such as flumazenil.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The term pen injector as used herein, refers to either a multidose pen injector capable of delivering one or more medicinal doses, or refers to a single delivery bolus autoinjector that is capable of delivering only one medicinal dose.

The term “subject,” as used herein, generally refers to a human. The subject can be a healthy individual, an individual that has or is suspected of having a disease or a pre-disposition to the disease, or an individual that is in need of therapy or suspected of needing therapy. The subject can be a patient. The subject may have or be suspected of having a disease.

The term “patient” or “subject in need thereof”, as used herein, generally refers to a person who is receiving or is expected to receive treatment. For example, a patient can be a person who has been prescribed a dosage regimen of a drug formulation comprising ketamine.

The term “user,” as used herein, generally refers to a person who uses or operates a system, device, or application described herein. The user can be a doctor or medical practitioner who configures the drug delivery device or dosage regimen(s). In some embodiments, the user is an authorized user who provides authentication information (e.g., authorization code or biometrics) to unlock the device or otherwise gain access to the dosage regimen settings. The user can be a subject who uses the drug delivery device to administer a dose according to the dosage regimen. The subject who self-administers doses of the drug formulation is generally not able to configure the dosage regimen.

The term “substantially pure,” as used herein, generally refers to a purity of at least 90% or higher. In some embodiments, a substantially pure substance has a purity of at least 90%, 91%, 92%, 93%, 94%, 95%, 96%, 97%, 98%, 99%, or more.

The term “tamper resistant,” as used herein, generally refers to having one or more features designed to mitigate the risk of tampering or interfering with the normal functioning of a system, device, or method described herein.

A “therapeutically effective amount” or “effective amount,” as used herein, generally refers to the amount of a pharmaceutical agent required to achieve a pharmacological effect. The term “therapeutically effective amount” includes, for example, a prophylactically effective amount. An “effective amount” of an NMDA receptor antagonist, such as ketamine, is an amount effective to achieve a desired pharmacologic effect or therapeutic improvement. The effective amount of an NMDA receptor antagonist, such as ketamine, will be selected by those skilled in the art depending on the particular patient and the disease level. It is understood that “an effective amount” or “a therapeutically effective amount” can vary from subject to subject, due to variation in metabolism of an NMDA receptor antagonist, age, weight, general condition of the subject, the condition being treated, the severity of the condition being treated, tolerance of side effects, and the judgment of the prescribing physician.

“Treat” or “treatment” as used in the context of a physical, neurological and/or psychiatric disorder refers to any treatment of a disorder or disease related to the symptoms of the physical, neurological and/or psychiatric disorder, such as stopping or reducing the symptoms of the disease.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain NMDA receptor antagonists of the present disclosure can exist in unsolvated forms as well as solvated forms, including hydrated forms. In general, the solvated forms are equivalent to unsolvated forms and are encompassed within the scope of the present disclosure.

“Pharmaceutically acceptable excipient” and “pharmaceutically acceptable carrier” refer to a substance that aids the administration of a compound, such as an NMDA receptor antagonist like ketamine, to and absorption by a subject and can be included in the compositions of the present disclosure without causing a significant adverse toxicological effect on the patient.

An “effective amount” is an amount sufficient for a compound to accomplish a stated purpose relative to the absence of the NMDA receptor antagonist (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side effects. Determination of the proper dosage for a particular situation is within the skill of the practitioner. Generally, treatment is initiated with smaller dosages that are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means intravenous, parenteral, intraperitoneal, intramuscular, or subcutaneous administration, or the implantation of a slow-release device, e.g., a mini-osmotic pump, to a subject. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, etc. By “co-administer” it is meant that a composition described herein is administered at the same time, just prior to, or just after the administration of one or more additional therapies (e.g., a benzodiazepine, a selective serotonin 5-HT3 receptor antagonist, a beta-blocker, and/or an inhibitor of CYP2B6 and/or CYP3A and/or CYP2C9). The compound (e.g., drug or active ingredient such as ketamine) of the present disclosure can be administered alone or can be co-administered to the patient. Co-administration is meant to include simultaneous or sequential administration of the compound individually or in combination (more than one compound or agent). Thus, the compositions can also be combined, when desired, with other active substances (e.g., to reduce metabolic degradation).

By “co-administer” it is meant that a composition described herein is administered at the same time, just prior to, or just after the administration of one or more additional therapies. The compounds of the present disclosure can be administered alone or can be co-administered to the patient. Co-administration is meant to include simultaneous or sequential administration of the compounds individually or in combination (more than one compound).

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side effects. Determination of the proper dosage for a particular situation is within the skill of the practitioner. Generally, treatment is initiated with smaller dosages that are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating drug dependence, psychiatric or neurological disorder, or pain disorders.

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

### Drug Delivery Devices Generally

In some aspects, disclosed herein are systems, devices, and methods for administering a drug formulation, which may be according to one or more programmed dosage regimen. The dosage regimen may be programmed or otherwise labeled or indicated in the component comprising the drug reservoir. For example, an RFID tag on a cartridge containing the drug formulation may include or be associated with information regarding the dosage regimen such as maximum bolus amount, total amount of drug that can be delivered over a time period. Accordingly, the dosage information may be obtained directly from the label, for example, an RFID tag containing the full dosage regimen. Alternatively, dosage information can be obtained indirectly from the label, for example, a cartridge tag or label specifies a serial number or identifier that is then used to locate a corresponding dosage regimen by the computer or computer processing component of the drug delivery device. In a non-limiting embodiment, the drug formulation comprises ketamine. In some embodiments, the system or device comprises a pump for administering the drug formulation. In some embodiments, the drug delivery device is a pen injector.

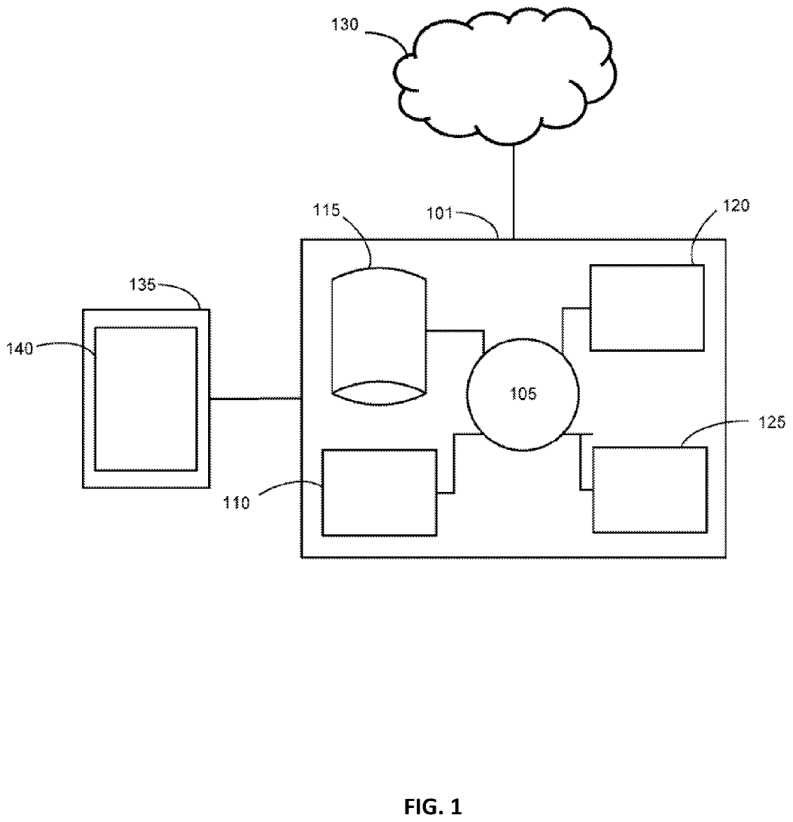

In some embodiments, a drug delivery device comprises a computer or computer system 101 as shown in FIG. 1. In some embodiments, the computer system comprises at least one processor 105 configured to carry out executable instructions to create a software application comprising one or more software modules 125 and configured for administering a dose of a drug formulation according to a programmed dosage regimen. In some embodiments, the drug delivery device comprises a memory 110, an electronic storage unit 115 (e.g., hard drive), a network adaptor or element for wired and/or wireless communications 120 with a network and/or cloud or a wireless communication device (e.g., mobile phone) 130. In some embodiments, the application comprises a control module for configuring at least one dosage regimen according to instructions provided by a user. In some embodiments, the control module operates a pump mechanism to deliver a dose according to at least one programmed dosage regimen. In some embodiments, the control module operates the pump mechanism to deliver a dose selected by a user. In some embodiments, the control module limits or restricts the dose based on one or more dose limits. In some embodiments, a dose limit is set by an authorized or administrative user (e.g., a doctor or medical practitioner). In some embodiments, an authorized user is recognized based on entry of an authentication code or other authenticating information (e.g., biometrics).

In some embodiments, the drug delivery device has an authentication code (e.g., a password) whose entry allows configuration of the dosage regimen and/or dosage limit(s). In some embodiments, the drug delivery device logs user activity relating to changes in at least one dosage regimen and/or dosage limit(s) such as changes made and/or time of change. In some embodiments, the drug delivery device logs every instance the authentication code was entered such as the time and/or place. In some embodiments, the logged information is uploaded over a network to a remote server for storage. In some embodiments, the remote server is accessible by the authorized or administrative user to view and/or download the logged information. In some embodiments, a drug delivery device comprises a software application comprising a monitoring module allowing an authorized user (e.g., a physician for the subject) to remotely monitor at least one dosage regimen over a network. In some embodiments, the monitoring module provides usage data to a remote server that is accessible by the authorized user. In some embodiments, the monitoring module transmits usage data directly to a communication device of the authorized user (e.g., without using an intermediary or remote server). In some embodiments, the software application comprises a remote access module allowing an authorized user to remotely configure or modify at least one dosage regimen over a network. In some embodiments, the remote access module allows an authorized user to login and configure/re-configure the drug delivery device remotely such as over a network. For example, a subject may call his physician asking for a change to the dosage regimen, and the physician may remotely configure the new dosage regimen. In some embodiments, the remote access module allows an authorized user to unlock the drug delivery device remotely such as over a network. In some embodiments, the remote access module communicates with the authorized user over a Wi-Fi, Bluetooth, cellular connection, or a combination thereof.

In some embodiments, the drug delivery device comprises an unlocked mode during which the at least one dosage regimen can be configured and/or modified (e.g., by an authorized user). In some embodiments, the drug delivery device comprises a locked mode during which the at least one dosage regimen cannot be configured and/or modified (e.g., when the device is being used by a subject who is not an authorized user to self-administer and/or alter a dose). In some embodiments, the drug delivery device requires input of an authentication code such as one provided by a doctor or other healthcare provider in order to switch between a locked mode and an unlocked mode. In some embodiments, the drug delivery device switches from an unlocked mode to a locked mode after receiving user input to switch to the locked mode. In some embodiments, the drug delivery device switches from an unlocked mode to a locked mode after receiving user input to switch to the locked mode and input of an authentication code.

Alternatively, in some embodiments, the drug delivery device is locked by the manufacturer after being configured with at least one pre-programmed dosage regimen. In some embodiments, the drug delivery device cannot be unlocked after being locked by the manufacturer such that even a healthcare provider for the subject is unable to reconfigure the at least one dosage regimen (e.g., device is permanently locked). In some embodiments, this permanent lock prevents abuse of the drug delivery device whereby the subject gains access to an authentication code of the healthcare provider by foreclosing the possibility of anyone being able to change the dosage regimen.

In some embodiments, the drug delivery device comprises a pump or injection mechanism configured for administering the drug formulation. In some embodiments, the pump or injection mechanism is configured for pumping or pushing a fluid such as a fluid drug formulation (e.g., ketamine or other controlled substance). In some embodiments, the pump or injection mechanism is configured to couple with a reservoir for storing the drug formulation. In some embodiments, the pump mechanism is configured to detachably couple with a cartridge for storing the drug formulation. In some embodiments, the cartridge is reusable. In some embodiments, the cartridge is disposable. In some embodiments, the cartridge is a single-use disposable cartridge. In some embodiments, the cartridge is configured to be tamper resistant.

In some embodiments, the drug delivery device comprises a user interface 135 allowing a subject to self-administer a dose of the drug formulation according to at least one programmed dosage regimen. In some embodiments, the user interface comprises a display screen or other display element (e.g., light bar and/or indicator or status lights) 140. In some embodiments, the user interface comprises at least one interactive element for receiving user input. In some embodiments, an interactive element is a physical interactive element such as, for example, physical buttons, knobs, dials, switches, toggles, wheels, click wheels, keyboard, or any combination thereof. In some embodiments, a user interacts with an interactive element by touching, tapping, swiping, twisting, turning, clicking, or pressing the element. In some embodiments, a user interface comprises one or more physical interactive elements (e.g., hard buttons). In some embodiments, a physical interactive element is a power button, a volume toggle button, a home button, a back button, menu button, navigation button(s), return button, multi-tasking button, camera button, a button on a physical keyboard, or any other physical button on the device. In some embodiments, the user interface comprises a display screen showing information about the dosage regimen and/or the current dose. In some embodiments, the display screen is an interactive touchscreen. In some embodiments, the user interface comprises a display screen showing information about the dosage regimen and/or the current dose. In some embodiments, a user interacts with the display screen using one or more physical interactive elements. In some embodiments, a user interacts with the display screen using one or more non-physical interactive elements (e.g., soft buttons on a touchscreen). In some embodiments, the user interface presents a user with one or more command options. In some embodiments, the one or more command options include at least one of administering a bolus of the drug formulation, commencing a continuous infusion of the drug formulation, pausing or canceling a dose, accessing an activity log (e.g., record of doses administered), accessing a dosage regimen (e.g., for review or for configuration depending on user authorization), and accessing device settings.

In some embodiments, the drug delivery device comprises at least one network element for carrying out wireless communications. In some embodiments, the at least one network element comprises a radio transceiver for communicating wirelessly over radio waves. In some embodiments, the at least one network element comprises a Bluetooth transceiver for communicating with one or more Bluetooth-enabled devices (e.g., a smartphone, a Bluetooth beacon, etc.). In some embodiments, the at least one network element comprises a WiFi transceiver for communicating with one or more WiFi-enabled devices (e.g., a WiFi router, a smartphone). In some embodiments, a network element communicates over a network using short-range communications with network or communication devices in close proximity (e.g., a personal area network). Examples of technologies that utilize short-range network communications include wireless headsets or earbuds and wireless wearable sensors (e.g., Fitbit). Short-range wireless technologies include communications standards such as ANT, UWB, Bluetooth, ZigBee, and wireless USB. In some embodiments, a drug delivery device uses short-range wireless technologies to communicate with a nearby device such as a subject's smartphone, which then optionally communicates or relays the communications to a remote authorized user. In some embodiments, the drug delivery device communicates using Wi-Fi and/or a cellular network (e.g., 2G, 3G, or 4G networks) to send and receive communications. In some embodiments, the drug delivery device establishes a communication channel with a communication device such as by “pairing” with the device. In some embodiments, the drug delivery device establishes an ongoing or temporary communication session with a communication device. In some embodiments, the communication session comprises data transfer between the drug delivery device and the communication device.

In some embodiments, the communication device comprises a processor that executes instructions to create a software application allowing monitoring and/or uploading of data from the drug delivery device. In some embodiments, the software application comprises a data module storing usage data for the device. In some embodiments, the data module stores information for doses administered by the subject. In some embodiments, the data comprises information on access times such as when the device has been accessed, who accessed the device (e.g., authorized or unauthorized user, subject or healthcare provider), doses administered (time, administered amount, administration rate, duration of administration, dosage number according to the dosage regimen, etc), user information (e.g., name, age, address, etc). In some embodiments, the data is stored on the drug delivery device. In some embodiments, the data is transmitted to the communication device. In some embodiments, the data is sent to a remote server. In some embodiments, the data is provided to the authorized user and/or healthcare provider for the subject. In some embodiments, the data is encrypted. In some embodiments, the data is sent via encrypted data channel(s). In some embodiments, the data is subject to 128 bit or 256-bit encryption. In some embodiments, the data is sent as encrypted files over one or more encrypted channels. In some embodiments, the remote server is part of a HIPAA compliant data center. In some embodiments, the remote server is HIPAA compliant. In some embodiments, the drug delivery device data storage (e.g., hard drive) has file/folder encryption, full disk encryption, or both. In some embodiments, data encryption is carried out according to the Advanced Encryption Standard (AES) for encryption.

In some embodiments, the application comprises a communication module configured to communicate wirelessly with a remote authorized user (e.g., using a network element). In some embodiments, the communication module allows messages or requests to be sent by the user of the drug delivery device to the remote authorized user (e.g., requesting a change to the dosage regimen and/or dosage limit). In some embodiments, the communication module is configured to receive instructions configuring or modifying at least one dosage regimen and/or dosage limit from the remote authorized user. In some embodiments, communications are provided to a remote authorized user indirectly by transmission to a server or communication device accessible by the remote authorized user. In some embodiments, the communication device is a computer, tablet, or phone accessible by the remote authorized user. In some embodiments, the server makes the communications available to the remote authorized user via a web application programming interface (API) that can be accessed by an Internet-enabled electronic device. In some embodiments, communications are provided to a remote authorized user via SMS (short message service), MMS (multimedia messaging service), email, or a chat application (e.g., Google chat, instant messenger, etc.).

Some embodiments of the present disclosure relate to various ways to create a reusable, delivery device. In some embodiments, the delivery device is waterproof. Past solutions range from throw away devices to very expensive and large pump systems. The mechanical sealing of a system has been difficult with removable power systems and cords and communications. The media storage and delivery is also a key problem in past systems and control and authentication thereof. Accordingly, the present disclosure enables simple, reliable solutions that provide a more positive outcome. For example, past wearable solutions are not designed for waterproof use and typically are not designed for everyday use. In addition, other delivery devices on the market actually enclose all of the components and require the user to dispose of the system, which increases overall cost. Size and portability has also been limited. Therefore, embodiments of the present disclosure include a cartridge system allowing better cleaning and ease of use.

In some aspects, disclosed herein are methods of sealing the system, device, and/or cartridge. In some embodiments, provided herein is an ultrasonically sealed enclosure that creates a completely sealed device. In some embodiments, a vent (e.g., GoreTex vent) is provided to allow flexing within pressurized altitudes and temperature changes while preventing moisture from entering. For instance, exposure of a delivery device to hot outdoor environments and cold environments can create pressure changes that the vent could protect against while limiting moisture from entering maintaining the structure and waterproof solution.

In some embodiments, disclosed herein are systems, devices, and methods for monitoring and providing feedback of safety parameters and patient pain rankings. This addresses a problem with securing the delivery material and the device. In some embodiments, the physician provides a prescription, for which the dosage/treatment regimen is monitored including recording the method and/or measurement parameters. Thus, the present disclosure provides methods to track and learn from each user for a prescription and optionally ranks the propensity for patient reactions and functionality (e.g., responsiveness, efficacy of treatment in reducing pain) to a given regiment.

In some aspects, disclosed herein are drug delivery devices. In some embodiments, these drug delivery devices are wearable devices configured to be worn or attached to the body, garment, or other worn equipment of a user (e.g., clipped to a belt, worn on a wrist band, etc.). Embodiments of these wearable devices provide several key solutions to past problems that have been observed and modified for better results in the wearable environment. For example, one challenge in the wearable device space is that the patient is expected to wear a device with and function in life normally. In some embodiments, the wearable device is configured to understand its environment and/or usage to enhance performance and/or understand its own function. For example, being in water or in wet environments and understanding when this is happening is important. In some embodiments, the wearable device comprises conductive and capacitive electrodes configured to monitor the relationship to the body, for example, in which the conductive portion monitors skin resistance. In some embodiments, one or more sensors allow decisions to be made based on sensor data. In some embodiments, sensor data is analyzed to determine the presence of a wet environment. In some embodiments, the wet environment is a wet environment external to a user or subject using the wearable device. In some embodiments, sensor data is analyzed to determine the presence of sweat or perspiration. In some embodiments, the sensor data comprises position as it relates to the body such as, for example, position/location of the sweat or perspiration. In some embodiments, the user is verified by the measurement of impedance between the two electrodes thus verifying to the delivery device control that the patient (e.g., the user) is present and the system is connected when that signal is connected and combines with the capacitive sensor detecting the body mass. In some embodiments, the user is verified with a mobile device ID, a cartridge ID, and their registration to the patient and/or physician. In some embodiments, this unit sends an ID code to the mobile device. In some embodiments, the mobile device is connected to a database such as a database stored on the cloud. In some embodiments, the mobile device is connected to the internet. In some embodiments, the connection utilizes an RF signal. In some embodiments, the link between the mobile device and the wearable device is BTLE, and a cellular link connects the mobile device to the database via the internet. Alternatively, the RF signal is a proprietary server frequency for additional security with a proprietary hub retained within the patient household. In some embodiments, data such as user statistics, processing pain, safety statistics, or any combination thereof are retained and measured over time. In some embodiments, the user charges the delivery device wirelessly until the device is fully charged by placing it on a charging device. In some embodiments, the user pairs and authenticates the mobile device and mobile application via database and/or network authentication. In some embodiments, the user inserts the cartridge and the system verifies and authenticates if the cartridge is valid or if it has been tampered with. In some embodiments, the wearable delivery device and system is authorized using first the database registrations for the cartridge ID, patient ID and password, patient mobile device Mac address ID, the delivery device ID, various device and cartridge security challenges (e.g., security challenge questions), present level and usage data, or any combination thereof. Once authenticated, in some embodiments, the database provides the prescribed delivery options, timing and delivery options. In some embodiments, the device is prepared for skin placement and optionally begins by running a portion of the delivery material. In some embodiments, the cannula moves past the septum and delivers a small amount of fluid. In some embodiments, the position of the cannula after moving past the septum indicates a valid cartridge and/or tampering or past usage, which are optionally stored on the RFID tag as dosage is delivered and past positions are logged on the cartridge. In some embodiments, the device is positioned or attached on the skin with adhesive, straps, elastic bands or other viable mechanical means. In some embodiments, one or more sensors detect the body and optionally set a body contact flag. In some embodiments, the mobile device (e.g., smartphone) authorizes and/or enables automatic cannulas insertion. In some embodiments, the patient presses a button on the mobile software application to cause instructions to be sent to the gear drive of the delivery device to insert the cannulas. In some embodiments, the cannula insertion is verified by using a tiny magnet that moves with the cannula's insertion body in which a Hall Effect semiconductor indicates a proper insertion position. In some embodiments, the protocol (e.g., a dose of a dosage regimen) is then run for that user unless the cartridge or device is removed. In some embodiments, the device delivers the full volume of the cartridge over the prescribed time, or some fraction of its full capacity as prescribed by the physician according to the dosage regimen. In some embodiments, the device allows a user to request additional dosage as it tracks pain level (e.g., user provides feedback on pain level during treatment). In some embodiments, the device allows a user to request additional dosing in either bolus or an increased basal rate (e.g., increasing the infusion rate of the drug). In some embodiments, the device allows a variety of protocols to be entered and administered. In some embodiments, the drug composition or formulation can be formulated to the desired effect based on dosages and delivery volumes of the device. In some embodiments, the pump is a gear drive screw drive. In some embodiments, the pump comprises a sensor configured to track plunger position. In some embodiments, the screw drive comprises a threaded rack molded in plastic that can be flexed about the inner package to accommodate smaller spaces and guided with plastic molded details to form a half loop and utilize a gear drive. In some embodiments, the screw drive is a blade that has a gear drive on one side that can flex about its thin side to enable a flexible rack drive. In some embodiments, the device comprises factor settings that are calibrated to retracted, started, pushed, completed volumes, or any combination thereof. In some embodiments, the device comprises a controller configured to monitor the one or more sensors for body contact, time using a real time clock, status of the cartridge, or any combination thereof. In some embodiments, the device comprises at least one accumulator configured to accumulate dose for the cartridge (or any other component configured to hold or store the drug formulation) first locally and optionally then stores the usage on the cartridge. In some embodiments, the at least one accumulator stores the usage on the cartridge via RFID after every dose so the cartridge has usage data to confirm dosing and authenticated usage. RFID tags can be read-only chips (e.g., information on the RFID chip cannot be altered after manufacture) or read-write chips that allow new information to be added to the tag and/or write over existing information. In some embodiments, the cartridge stores use by dates, patient ID, device ID, mobile ID, dose start date, removal flag, pain scales, or any combination thereof as authenticators for preventing tampering and reuse. In some embodiments, the device comprises tramper resistance features related to sealing the drug reservoir within the pump in such a manner that it is difficult to access the medication contained within the reservoir either, after it is coupled to the pump by the user, or after it is coupled to the pump by the manufacturer, pharmacy, clinician or other certified person. In some embodiments, the reservoir has a sliding window that is moved to cover the medication fill port after the reservoir is loaded. In some embodiments, the reservoir rotates to hide the fill port internally. In some embodiments, the reservoir is completely sealed inside the device after it is filled at the factory, the pharmacy, the clinician office or other certified location.

### Pen Injector Configuration

In some aspects, a drug delivery device disclosed herein is configured as a pen injector. The pen injector design is easy to use and can be configured with tamper resistant features and dosage control, including any of the features disclosed herein. In some embodiments, the pen injector disclosed herein is designed for intramuscular or subcutaneous bolus administration by the patient outside of the clinical environment. The pen injector is configured to be tamper resistant, wherein the design makes it difficult or impractical to remove the medication using conventional means such as with a syringe or other medication transfer devices. This helps address the risk of allowing self-operated drug delivery devices with high value or controlled medicinal formulations, or medicines with a high safety risk profile associated with dosage control or misuse (e.g., ketamine).

In some embodiments, the pen injector is configured as a single-part (i.e., one integrated device) prefilled and preloaded (e.g., fixed bolus amount) device. In some embodiments, the pen injector is configured as a two-part prefilled and preloaded (e.g., fixed bolus amount) device. The two-part configuration can include a separate prefilled and preloaded pen injector body and a reusable pen injector driver. The pen injector body contains the reservoir holding the drug formulation and may be disposable after use (e.g., after the reservoir is depleted), while the pen injector body holds any electronics, the user interface, and the drive system. The single-part configuration is simpler and easier to use, while the two-part configuration may reduce cost and produce less waste (e.g., only the disposable component is disposed, while the pen injector body can be reused).

In some embodiments, the pen injector or autoinjector is designed to deliver a single bolus including all or partial contents stored within the reservoir. The reservoir can be a prefilled and preloaded cartridge or prefilled and preloaded staked needle syringe. The autoinjector can be configured to deliver a single bolus of all or partial contents stored within the reservoir, after which it is no longer be capable of delivery. The single bolus delivery could be activated by button press on top of or around the body of the autoinjector. In some embodiments, the single bolus delivery could be activated by the patient-contacting surface or the needle-shield only. In some embodiments, the single bolus represents the total deliverable volume within the reservoir. In some embodiments, the single bolus represents a partial amount of the deliverable volume within the reservoir. In some embodiments, the amount delivered is determined and set by the patient or healthcare provider. For example, the single bolus autoinjector can incorporate a dial to set a single bolus amount between a range of options. In one embodiment, the range is represented as Low, Medium, and High. In some embodiments, the range of options provides selectable values. Non-limiting examples of the selectable values for a drug such as ketamine include 0.25 mg/kg, 0.5 mg/kg, 1.0 mg/kg, 1.5 mg/kg, and 2.0 mg/kg. Such doses can be administered in one or multiple injections, for example, three separate injections separated by an amount of time. In some embodiments, the multiple separate injections are separated by between 0.1 to 205 minutes. Alternatively, multiple separate injections can be delivered together without spreading them out over time. In some embodiments, the multiple separate injections are used to administer between 3 to 30 mg doses each. The settable bolus amounts could be any increment from the minimum dispensable amount to the maximum deliverable fill volume for the reservoir. In some embodiments, once the patient or healthcare provider selects a bolus dose amount and activates the delivery or delivery mechanism, the single bolus autoinjector becomes locked and can no longer deliver further doses or injections.

In some embodiments, the pen injector or autoinjector is configured to obtain authorization from a mobile device. For example, the mobile device could be a user smartphone that is communicatively coupled to the pen injector or autoinjector and has an app that causes the smartphone to send an unlock authorization signal to the pen injector (either wired or wireless) deactivating the locking mechanism (e.g., unlocking the cap and activation button to allow injection(s)). For reference herein, the term pen injector can be used to represent a single or multidose delivery device, or a single delivery bolus autoinjector that is capable of delivering only a single bolus.

In some embodiments, the pen injector comprises a readiness window and activation button. The readiness window can include an indicator of readiness of the pen injector to perform an injection. For example, the readiness window can have an LED that displays different colors that indicate the state of readiness such as blue for pairing with a mobile device, green for ready for injection, yellow for the device being locked out (e.g., no unlock authorization), red for no more injections available (i.e., reservoir is depleted or has insufficient amount of drug formulation for one more bolus), flashing red for device error, or any combination thereof. In some embodiments, the readiness window provides a graphical display showing the text, symbol(s), or icon(s) indicative of readiness to perform the injection. In some embodiments, the pen injector comprises a dial dose feature and/or a set dosage window. The dial dose feature can allow for the user to adjust the dosage for an injection, and the set dosage window can provide an indication of the dosage amount.

In one embodiment, the pen injector is configured as a prefilled and preloaded disposable pen injector with a defined amount of controlled medication contained within the device. An advantage of a prefilled and preloaded disposable pen injector is that it can be designed with the medicinal container packaged within the pen injector during manufacture such that there is no practical access to the medicine contained within the device primary container (i.e., single-part configuration). This tamper resistant design is important to protect against diversion or misuse of a high value or controlled substance such as ketamine. Additionally, in some cases, this pen injector design includes an integrated lockout feature preventing the pen from delivering the medication until the timing mechanism of the lockout feature is positioned to allow the injection to occur. This timing mechanism can be configured to be initiated after the first patient injection.

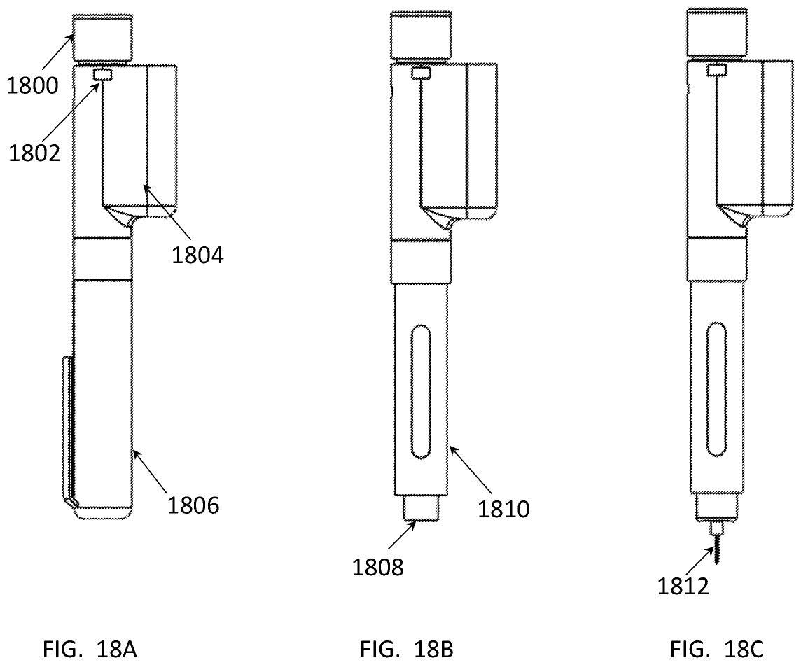

FIG. 18A and FIG. 18B show a non-limiting embodiment of single-part prefilled and preloaded (fixed bolus) pen injector. The pen injector is shown with an activation button 1800, a readiness window 1802, a time controlled injection and cap lock-out mechanism 1804, a pen injector cap 1806 (cap removed 1810 in FIG. 18B), and access to the cartridge septum 1808. FIG. 18C shows the injection needle 1812 extended after activation. FIG. 18A shows an example of the pen injector in an operational state with the injector cap installed but unlocked and ready to be removed (readiness indicator may show color indicating bolus injection is available to be delivered). FIG. 18B shows the pen injector with the cap removed. FIG. 18C shows the bolus injection ready to be delivered with the needle shield removed and the needle assembly 1812 attached and ready for injection.

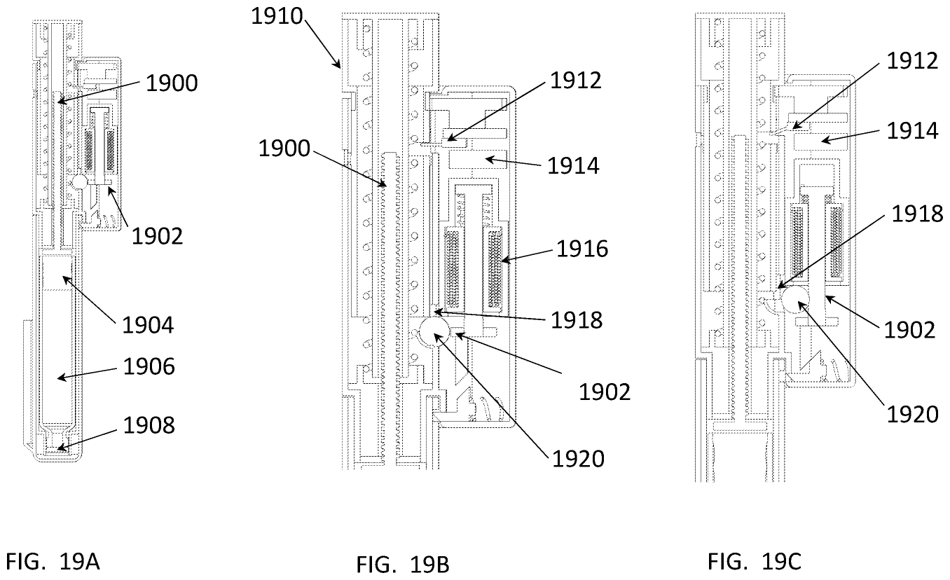

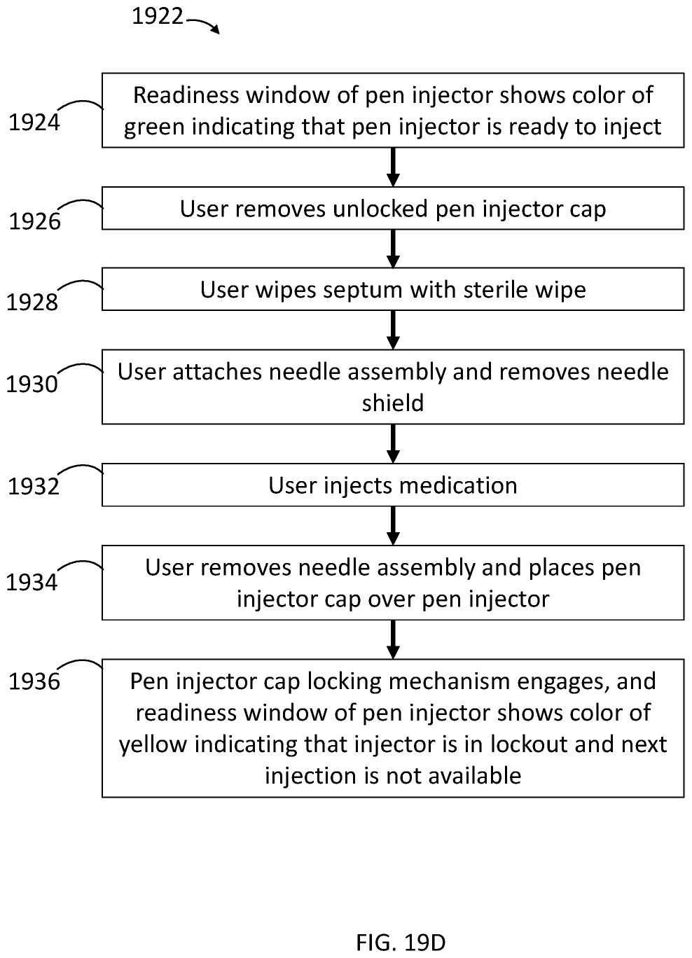

FIG. 19A shows a section view of the single-part prefilled and preloaded pen injector with the injection lock-out mechanism engaged 1902 to lock out the activation button. Also shown are the leadscrew 1900, cartridge plunger 1904, prefilled medication 1906, and the cartridge septum 1908. FIG. 19B shows a close-up section view with the activation sensor 1912 in the non-activation state. The electronic circuit (e.g., uP or ASIC), crystal oscillator, and battery are shown together 1914. Also shown is the electronically driven solenoid 1916, the activation button 1910 that is locked out from advancing 1918 due to the lockout feature 1920 modulated by the upward position of the solenoid 1902. FIG. 19C shows a section view with the injection and lock-out mechanism disengaged, thereby allowing the activation button to activate injection of the prefilled medication. The activation sensor 1912 is showing that the activation button has been depressed, thereby indicating to the internal electronic circuit 1914 that device activation has been activated. The downward movement of the solenoid allows the activation button lockout feature to release 1902, thus disengaging 1920 the lockout feature and allowing the activation button to be unlocked and ready to inject 1918. FIGS. 19D and 18A-18C show an operational sequence for the single-part prefilled and preloaded pen injector. FIG. 19D provides a flow chart for an operational sequence including one or more of: readiness window showing a green color indicating the pen injector is ready to inject 1924, removal of the unlocked cap 1926, wiping the septum with a sterile wipe 1928, attaching the needle assembly and removing needle shield 1930, injecting the medication 1932 with the lockout mechanism deactivated, removing the needle assembly and attaching injector cap 1934, and engagement of the pen injector cap locking mechanism with the indicator showing a yellow color indicating the injector is in lockout with the next injection being unavailable 1936.

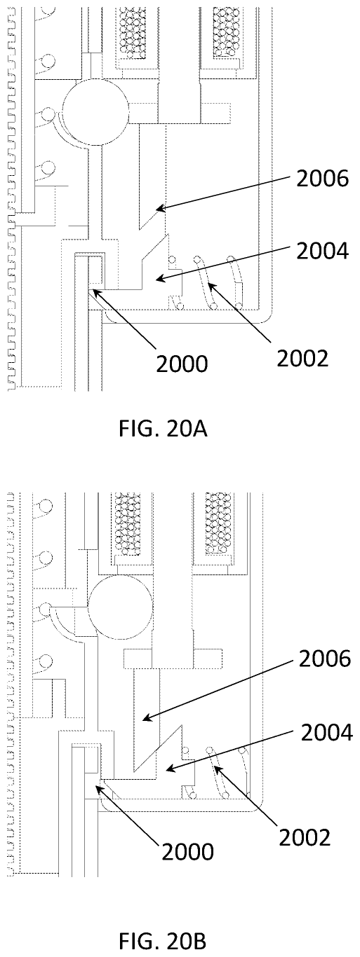

FIG. 20A shows a close-up section view of the lockout mechanism engaged to lock out the cap for a non-limiting embodiment of the single-part prefilled and preloaded pen injector. The solenoid driven cap unlock member is in the cap locked state 2006 in FIG. 20A with the cap lockout engagement pin engaged 2004, and the cap locked from being removed 2000. Also shown is the cap lockout return spring 2002. FIG. 20B shows a close-up section view of the lockout mechanism disengaged in which the solenoid driven cap unlock member is in a cap unlocked state 2006 in which the cap lockout engagement pin is disengaged 2004, and the cap is unlocked and able to be removed 2000.

The cap lockout feature can be a simple catch that is extended and captures a ridge feature inside the cap wall, and the catch can be released by a timer allowing removal. The cap lockout feature as shown in FIGS. 20A-20B allows for the pen cap to be replaced on the pen injector, following disposable pen needle removal, and automatically relocked and not removable again until a cap unlock signal is provided. In another embodiment, the pen injector remains in lockout after the first injection until the pen injector cap is replaced on the pen injector and the pen injector lockout feature is reengaged. This utility will ensure that only one injection is provided with each authorizing signal to remove the pen injector cap.

In some embodiments, the prefilled and preloaded pen injector is configured with either a variable patient settable bolus dose range (e.g., using a dial dose feature), for example from 5 to 300 microliters per dose in 5 microliter dose increments, or with a fixed bolus dose per activation such as 20 microliters per dose. As an example, for the administration of ketamine, a single bolus dose may be 25 microliters (1.75 milligrams) for a 70 mg/mL formulation concentration to provide the targeted effect. If the pen injector contains a total of 2.5 milliliters of a 70 mg/mL Ketamine formulation, the pen would be capable of delivering one hundred 25 microliter boluses.

In some cases, the prescribed treatment of several therapies requires the medication to be delivered by a controlled amount over time, such as hours or days, in order to not over-deliver and cause potential adverse events. For example, this is the case for insulin and ketamine. Over-delivery of insulin could result in hypoglycemia leading to several complications, including coma or death. Over-delivery of ketamine could lead to disorientation, dissociative effects, rhabdomyolysis, seizures, memory loss, sleep disorder, and chronic ulcerative cystitis. Therefore, the pen injector can be configured to provide for controlled delivery amounts over time by leveraging a lockout timing method (mechanical or electronic) that releases the opportunity for another injection over a time period. In the illustrative example of ketamine, the pen injector can be configured to limit the number of doses within a set period of time. For example, the pen injector can be configured such that only one 25 microliter injection could occur per 15 min period, or such that no more than four 25 microliter injections could occur per hour.

In some embodiments, the device, which can be a wearable injector or pump or a pen injector, is configured to limit the dosage regimen to no more than 1, 2, 3, 4, 5, 6, 7, 8, 9, or 10 doses within a time period of no more than 15, 30, 45, or 60 minutes, or within a time period of no more than 2, 3, 4, 5, 6, 7, 8, 9, 10, 11, 12, 13, 14, 15, 16, 17, 18, 19, 20, 21, 22, 23, or 24 hours, or within a time period of no more than 1, 2, 3, 4, 5, 6, or 7 days. In some embodiments, each dose is a bolus of about 75 microliters to about 200 microliters. In some embodiments, each dose is a bolus of about 75 microliters to about 100 microliters, about 75 microliters to about 125 microliters, about 75 microliters to about 150 microliters, about 75 microliters to about 175 microliters, about 75 microliters to about 200 microliters, about 100 microliters to about 125 microliters, about 100 microliters to about 150 microliters, about 100 microliters to about 175 microliters, about 100 microliters to about 200 microliters, about 125 microliters to about 150 microliters, about 125 microliters to about 175 microliters, about 125 microliters to about 200 microliters, about 150 microliters to about 175 microliters, about 150 microliters to about 200 microliters, or about 175 microliters to about 200 microliters. In some embodiments, each dose is a bolus of about 75 microliters, about 100 microliters, about 125 microliters, about 150 microliters, about 175 microliters, or about 200 microliters. In some embodiments, each dose is a bolus of at least about 75 microliters, about 100 microliters, about 125 microliters, about 150 microliters, or about 175 microliters. In some embodiments, each dose is a bolus of at most about 100 microliters, about 125 microliters, about 150 microliters, about 175 microliters, or about 200 microliters. In some embodiments, the concentration of the drug formulation (i.e., the active ingredient such as ketamine, a psychedelic, or other suitable compound described herein) is about 40 mg/mL to about 100 mg/mL. In some embodiments, the concentration of the drug formulation is about 40 mg/mL to about 50 mg/mL, about 40 mg/mL to about 60 mg/mL, about 40 mg/mL to about 70 mg/mL, about 40 mg/mL to about 80 mg/mL, about 40 mg/mL to about 90 mg/mL, about 40 mg/mL to about 100 mg/mL, about 50 mg/mL to about 60 mg/mL, about 50 mg/mL to about 70 mg/mL, about 50 mg/mL to about 80 mg/mL, about 50 mg/mL to about 90 mg/mL, about 50 mg/mL to about 100 mg/mL, about 60 mg/mL to about 70 mg/mL, about 60 mg/mL to about 80 mg/mL, about 60 mg/mL to about 90 mg/mL, about 60 mg/mL to about 100 mg/mL, about 70 mg/mL to about 80 mg/mL, about 70 mg/mL to about 90 mg/mL, about 70 mg/mL to about 100 mg/mL, about 80 mg/mL to about 90 mg/mL, about 80 mg/mL to about 100 mg/mL, or about 90 mg/mL to about 100 mg/mL. In some embodiments, the concentration of the drug formulation is about 40 mg/mL, about 50 mg/mL, about 60 mg/mL, about 70 mg/mL, about 80 mg/mL, about 90 mg/mL, or about 100 mg/mL. In some embodiments, the concentration of the drug formulation is at least about 40 mg/mL, about 50 mg/mL, about 60 mg/mL, about 70 mg/mL, about 80 mg/mL, or about 90 mg/mL. In some embodiments, the concentration of the drug formulation is at most about 50 mg/mL, about 60 mg/mL, about 70 mg/mL, about 80 mg/mL, about 90 mg/mL, or about 100 mg/mL. As an illustrative example, a 100-150 microliter bolus dose of a 70 mg/ml Ketamine formulation (7-10.5 mg ketamine) could be dosed once every 20-24 hours.

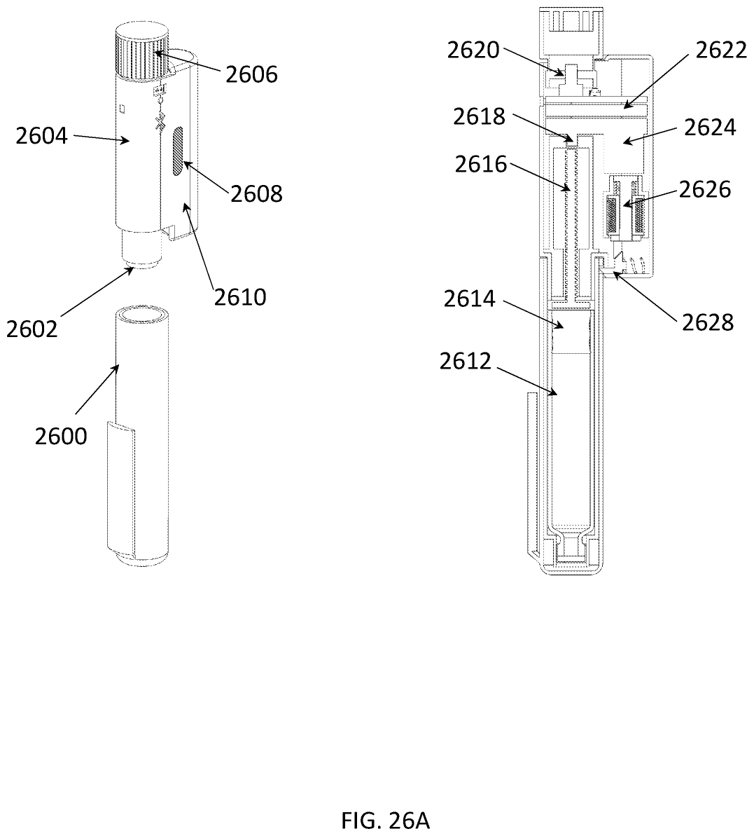

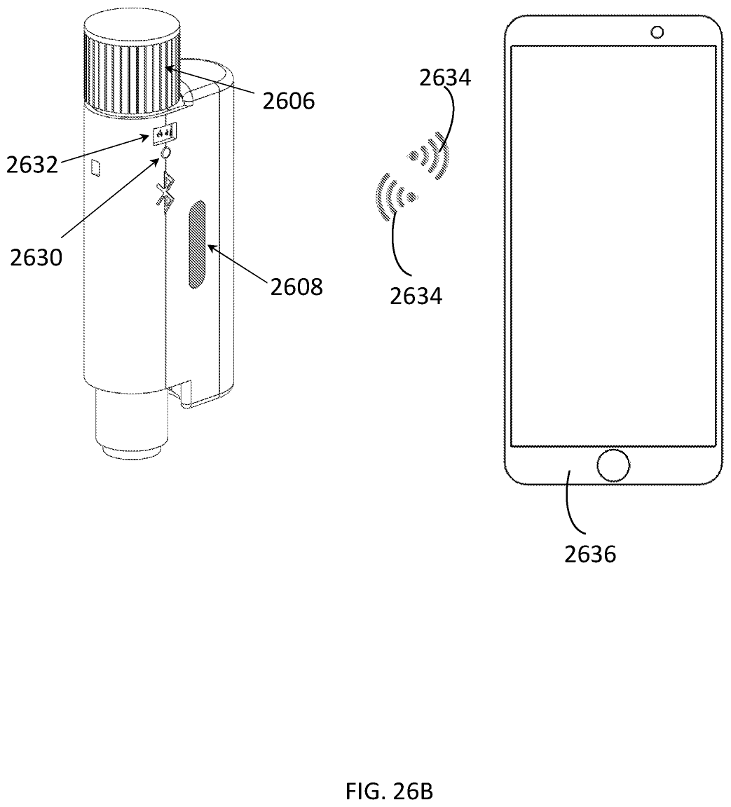

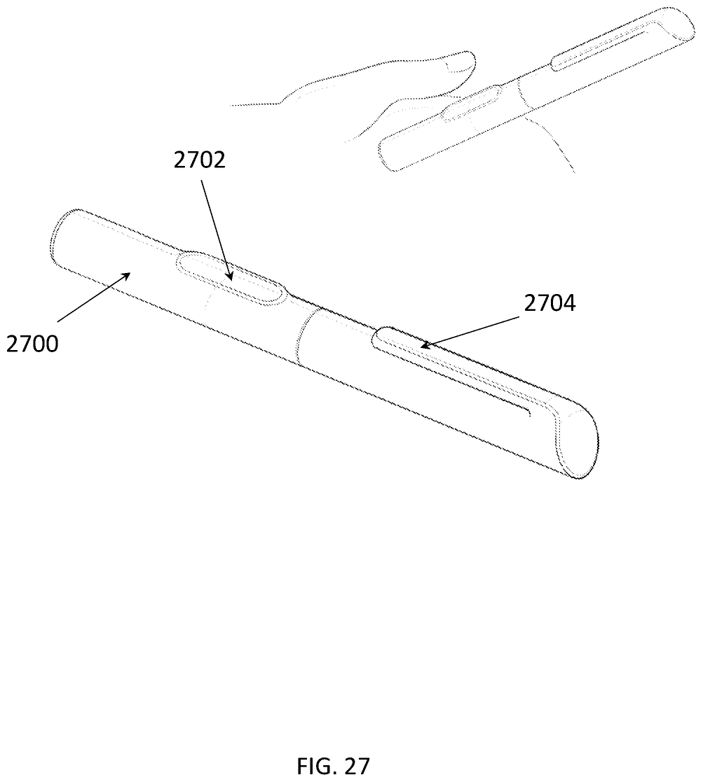

In some embodiments, the pen injector is configured to connect with a mobile device, for example, a user smart phone or tablet. The connection can be wired (e.g., USB/micro-USB connection) or wireless (e.g., Wi-Fi or Bluetooth). In some embodiments, authorization to inject the medication is provided at least in part through the mobile device. As an illustrative example, a mobile application installed on the mobile device provides authorization to the pen injector in response to user input (e.g., entry of password, biometric authentication). As an illustrative example, FIGS. 26-27 represent biometric authentication using a fingerprint scanner on a pen injector or autoinjector.

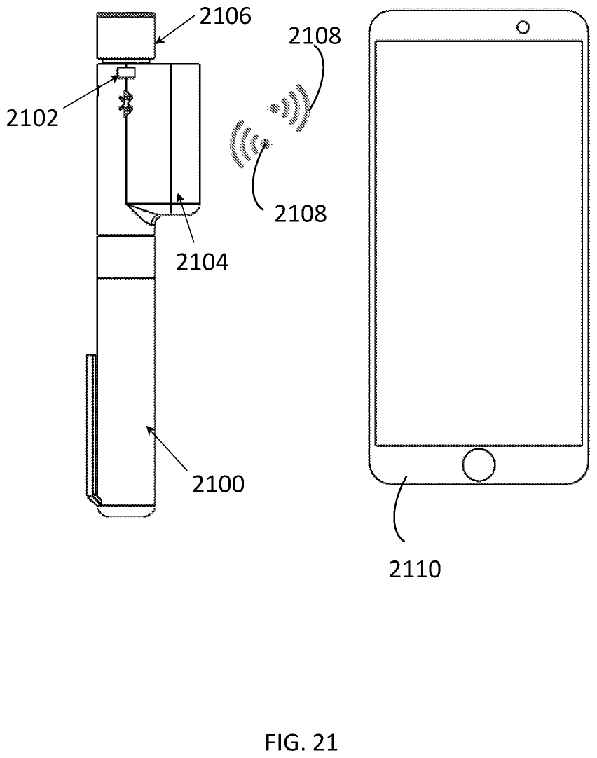

FIG. 21 shows a non-limiting embodiment of a single-part prefilled and preloaded (fixed bolus dose) pen injector with a connectivity interface. The pen injector is shown with the activation button 2106 and a readiness window 2102, which can show different indicators such as color corresponding to different states or readiness for injection (e.g., green for ready to inject, yellow indicating lockout period following injection, and/or red indicating no more injections). Also shown is the outside of the timer controlled injection and cap lockout mechanism 2104 and the pen injector cap 2100 locked onto the pen injector. The pen injector may communicatively connect to a mobile device 2110 via a wireless connection 2108.

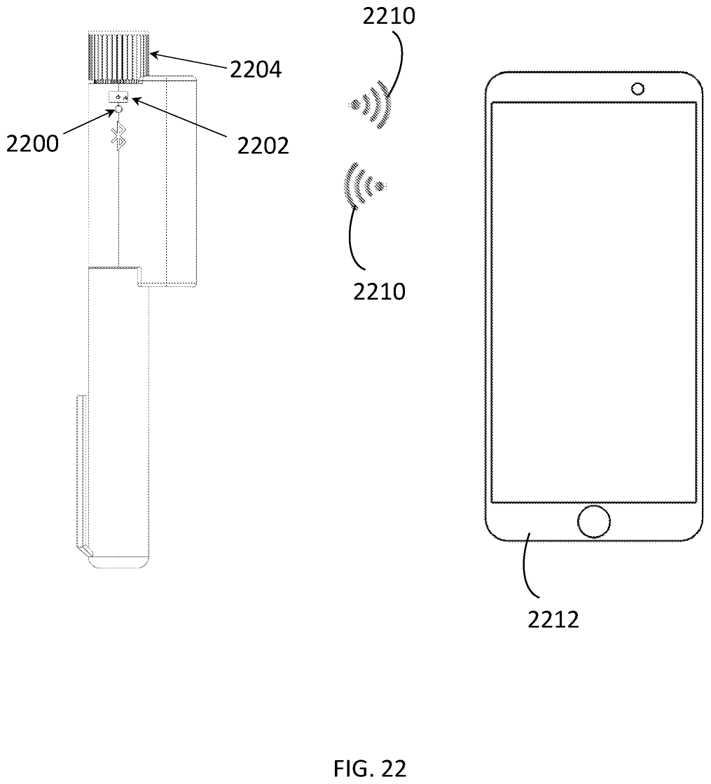

FIG. 22 shows a non-limiting embodiment of a single-part prefilled and preloaded pen injector with a patient adjustable dose feature (e.g., dial-a-dose) 2106 (can be both activation button and adjustable dose knob) with rheostat feedback and mobile authorization. The dose window 2202 and LED readiness indicator 2102 are shown along with the mobile device 2212 the injector can optionally connect to through a wireless connection 2210.

In some embodiments, the pen injector comprises a lockout mechanism that is purely mechanical (i.e., no electronic lockout). In the case of the mechanical only pen injector, the pen injector lockout timing control is accomplished by mechanical escapement, for example, a mechanical escapement used in a spring-driven mechanical timepiece. In some embodiments, the timing mechanism is initiated when the pen injector is activated for the first time by the user or patient. In this case, the timing mechanism can control a cam, pin, lever, slot, or similar element that inhibits additional bolus deliveries until the predefined time or time window is reached. After the predefined time or time window is reached such as, for example, 15 minutes in this example, the movement of the cam, pin, lever, slot, or the like allows for another single bolus (e.g., 25 microliter) to be delivered.

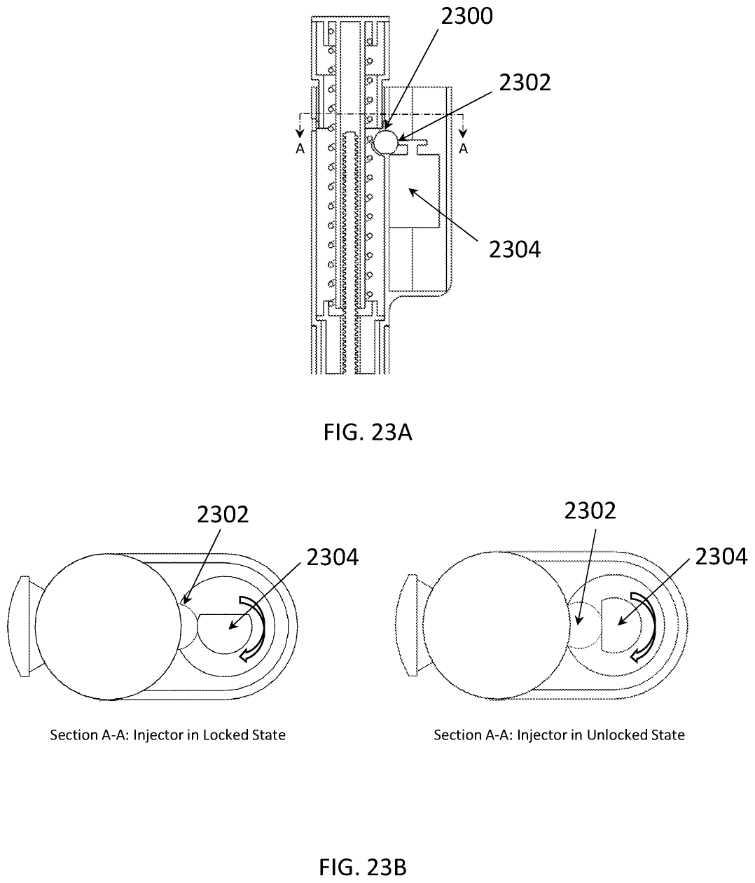

FIG. 23A shows a section view of a non-limiting embodiment of a single-part prefilled and preloaded pen injector with a lockout feature using a mechanical escapement allowing for one button activation. In this case, the mechanical escapement comprises a spring driven clockwork type mechanism that only enables unlocking after a specified amount of time has expired. For example, the lockout feature 2302 is driven by a spring-driven mechanical escapement (e.g., rotating cam) 2304 such that the activation button is locked out from advancing 2300. FIG. 23B shows a top-down view of the pen injector in a locked and unlocked configuration with the rotating cam 2304 in a locked state or unlocked state.

Accordingly, the mechanical lockout mechanism can provide timing-based control of drug delivery. This timing-based control can continue until the total contents of the prefilled preloaded container or reservoir is exhausted, or until a maximum allowable number of boluses are delivered. Alternatively, the timing mechanism can be configured such that only a defined set of boluses could be delivered over a defined period of time. For example, the timing mechanism could release up to four 25 microliter bolus injections over a period of one hour, and this could be reset each hour of time the pen injector is used or until the total contents of the prefilled preloaded container is exhausted, or until a maximum allowable number of bolus are delivered. This time-based control provides a lockout system to manage the number of allowable boluses over a defined time period to prevent misuse or delivery other than prescribed by a healthcare professional.

Alternatively, the prefilled and preloaded disposable pen injector can be configured to incorporate a simple electronic circuit that controls delivery timing as opposed to a mechanical only escapement, as described above. The prefilled and preloaded disposable pen injector can be configured as a single-part device so that the entire device is disposable. In one example, the electronic circuit includes an ASIC (Application Specific Integrated Circuit) and a crystal that is designed to control the timing of bolus releases through a lockout mechanism that controls a cam, pin, lever, slot, or similar element. The power to the electronic circuit can be initiated as a result of the first button press by the patient to deliver the first bolus dose. This action would thereby wake up the electronic circuit, including the ASIC and crystal, to properly control the timing of any additional bolus injections. Initiating power to the electronic circuit as a result of the first button press has the advantage of not draining the battery contained within the pen injector over the storage period prior to first use.

There are a multitude of methods to control the mechanical release lockout mechanism of a cam, pin, lever, slot, or similar element from an electronic-driven circuit, including electromagnetic motion (linear or rotary), shape memory alloys that undergo deformation due to changing temperature, electronically controlled motor, and other suitable mechanisms. For example, a simple ASIC time-controlled circuit can be coupled with an electromagnet to release a lever, thereby unlocking the device allowing for a single bolus injector injection to occur when the patient presses the activation button. Once the user or patient presses the activation button and delivers a bolus, the lockout period begins and a lever is reset and will inhibit another bolus from being delivered until the timing circuit again activates to release the lever. Examples of the lockout are shown in FIGS. 19A-19B, 20A-20B, and 23A-23B. Alternatively, in some embodiments, the pen injector is configured such that with each lever activation as controlled by the timing circuit, a set number of more than one bolus injections could occur before and until the lever is triggered again by the control circuit. An ASIC is one method to control the timing circuit; alternative methods include a microprocessor.

In some embodiments, to improve on the user experience of a pen injector, the spring-loaded activation button is configured to travel to near full deflection prior to releasing the bolus injection mechanism. In this design, the button works as a release to auto inject the single set bolus by a spring-driven mechanism. This, of course, assumes the timing circuit is set to allow an injection to be triggered.

In some embodiments, the prefilled and preloaded pen injector comprises a display providing use data, audio, and/or tactile feedback, or connectivity to a second device (e.g., a mobile device such as a smartphone) that shares data on pen usage. Furthermore, connectivity between the pen injector and a second device such as a mobile phone could be used to provide setting changes to the pen injector or additional unlocking codes as further tamper resistance. Illustrative examples of pen injectors configured to connect with a second device such as a mobile phone are shown in FIG. 21A and FIG. 22.

There are clear benefits of a prefilled and preloaded disposable pen injector that incorporates a mechanical only timing circuit or electronically controlled timing circuit. This type of design provides for simple setup and use out of the box while providing the tamper resistance and device lockout needed to control misuse and delivery other than prescribed. However, integrating a mechanical or electronically controlled timing circuit within a disposable pen injector adds additional cost and environmental considerations for the disposal. Alternatively, the pen injector could be made up of a system with two parts, 1) the disposable component (Pen Injector Body) that contains the prefilled medicinal container, and 2) the reusable component (Pen Injector Driver) that contains the electronics, user interface, and drive system. The reusable component could be coupled to the disposable component through magnetic coupling, mechanical drive shaft, rotary gear interface, or the like. Each provides tradeoffs and benefits. However, there is a benefit of decoupling the disposable component and the reusable component since the cost of the reusable component can be amortized over multiple uses of the disposable component. Additionally, there is an environmental benefit of reducing the need to dispose of the material in the reusable component with each completed disposable pen use. In order to maintain the tamper resistance of the two-part design, the Pen Injector Body disposable component is configured such that there is no practical means to access the primary container plunger resulting in unintended delivery or misuse. As an example, the disposable component comprises a magnetic coupling to the reusable system, thus providing the opportunity for a clean interface between the two sub-systems.

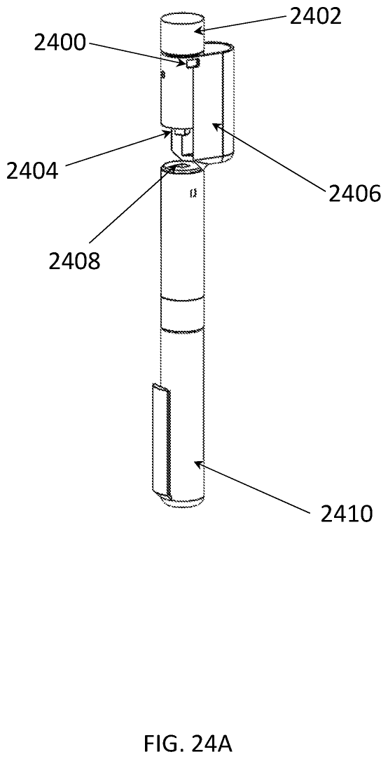

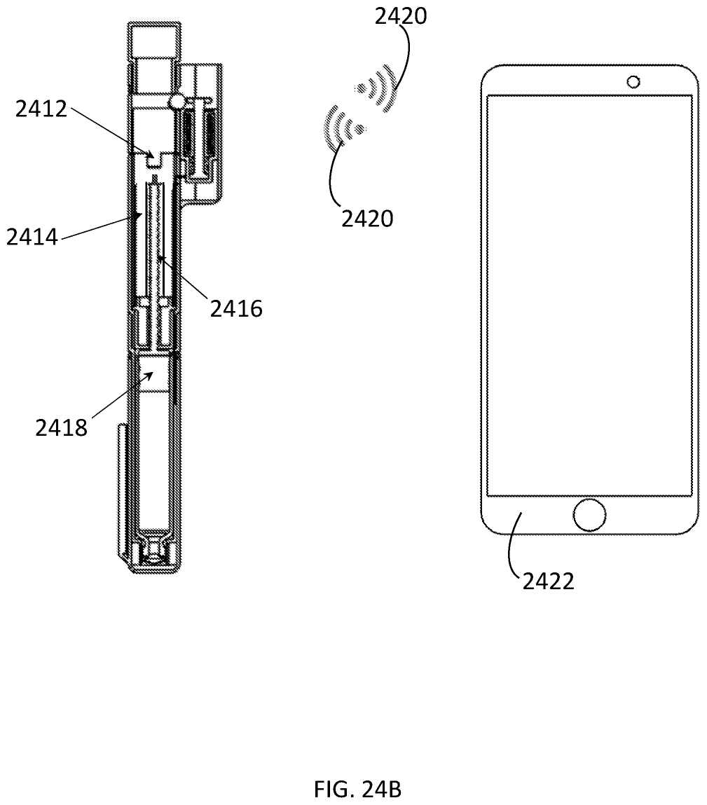

FIG. 24A and FIG. 24B show a non-limiting embodiment of a two-part prefilled and preloaded pen injector with a mechanical interface for coupling of the pen injector driver and the pen injector body. FIG. 24A shows the two-part pen injector with a reusable pen injector driver having a readiness window 2400, activation button 2402, coupling member 2404, and timer controlled injection and cap lock-out mechanism 2406, and a pen injector body having a coupling socket 2408 (to engage with the coupling member of the reusable pen injector driver), the drug reservoir (not shown), and reversibly detachable pen injector cap 2410. FIG. 24B shows the reusable pen driver 2412 coupled to the pen injector body and the internal rotating drive nut assembly 2414 and cartridge plunger 2418 in which the leadscrew advances the cartridge plunger due to drive nut rotation 2416. Also shown is the mobile device 2422 that is optionally connected to the pen injector using a wireless connection 2420. Alternatively, in some embodiments, the mobile device is connected to the pen injector or any other delivery system disclosed herein using a wired connection.

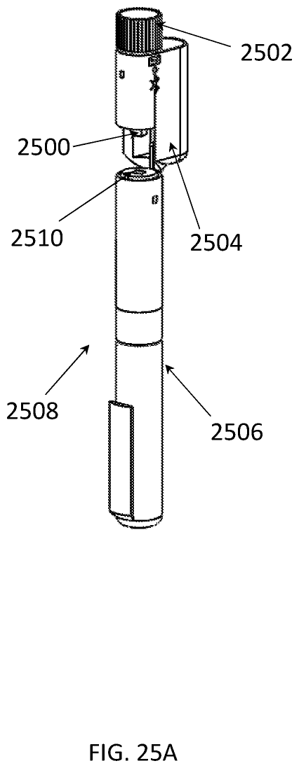

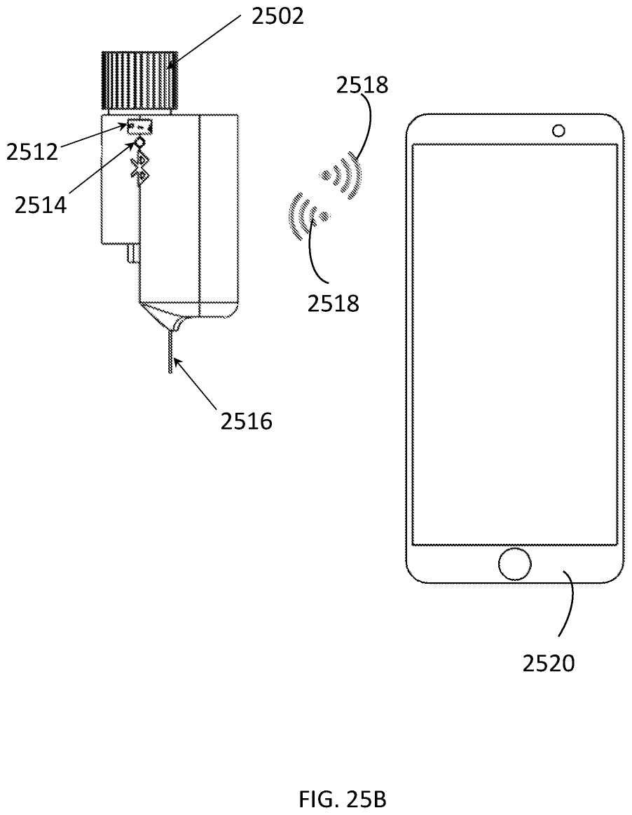

FIG. 25A shows a non-limiting embodiment of a two-part prefilled and preloaded pen injector body with a mechanical interface for coupling of the pen injector driver and the pen injector body and an interface comprising a patient adjustable dose feature (e.g., dial-a-dose knob). FIG. 25A shows the activation button and a dial-a-dose knob 2502 allowing a user to set the desired bolus dosage, the coupling member from the reusable pen injector driver 2500, the timer controlled injection and cap lockout mechanism 2504, the coupling socket in the pen injector body 2510, the pen injector body assembly 2508 and the portion of the body assembly containing the cap lockout feature 2506. FIG. 25B shows a close-up view of the dose set window 2512 and readiness LED indicator 2514 of a pen injector configured to communicate with a second device such as a mobile phone 2520 via a wireless connection 2518. Also shown is the cap locking feature 2516. In some embodiments, a magnetic coupling is used instead of a mechanical coupling (not shown).

In both the prefilled preloaded disposable pen injector with integrated electronic circuit (single-part design), and the two-part design that incorporates an electronically controlled reusable component, a finger/thumb print ID can be incorporated to unlock and enable delivery, protect against accidental delivery, and ensure that only the authorized patient is using the device over multiple injections as shown in FIGS. 26-27. Furthermore, in some embodiments, the pen injector provides connectivity to another device (e.g., wired or wireless connection), such as a mobile phone, to provide several additional benefits for a controlled delivery application, including leveraging the mobile device to ensure that only the authorized user unlocks the pen injector allowing for delivery. This provides an additional security level such that only the authorized and prescribed patient can have access to the high value or controlled medication, or medicines with a high safety risk profile associated with dosage control or misuse such as Ketamine or Insulin. Bidirectional connectivity also provides useful information to the healthcare provider on patient use of their medicine against the respective prescription. Some patient use data could include time, date, and amount of each delivered dose, and temperature, the orientation of the device at the time of each delivery. Additionally, by leveraging the mobile device as an interface, the physical location of the prefilled preloaded pen injector at activation and deliveries could be monitored as well. Illustrative embodiments of two-part pen injector devices that provide connectivity to another device are shown in FIGS. 24A-24B and FIGS. 25A-25B.

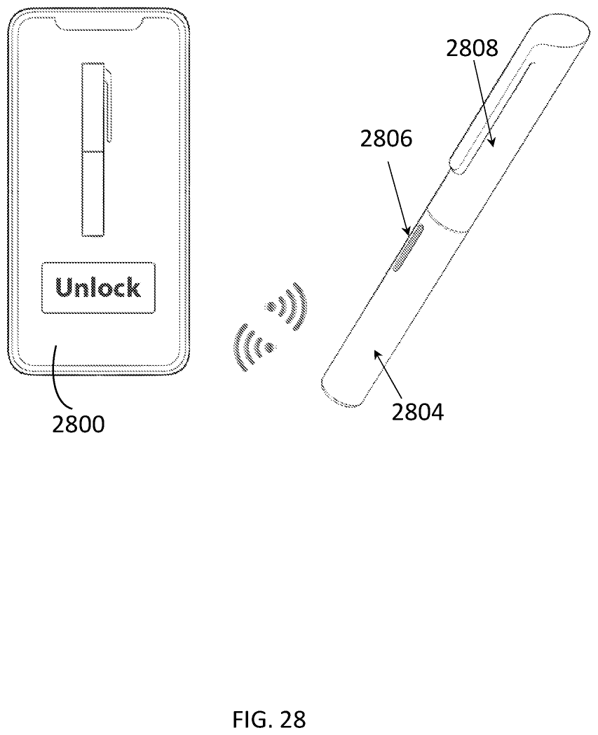

In some embodiments, the prefilled and preloaded pen injector (either single or two-part design with a reusable component) comprises a cap to protect the septum end of the device during shipping and between uses. The patient would remove this cap prior to use and attach a new sterile needle to the device and therefore puncturing the septum allowing delivery. In some embodiments, to provide further protection against unauthorized use, the electronically controlled pen injector comprises a cap at the distal end of the pen injector that is locked in place and is only removable if one or a combination of the following actions occur: 1) the device recognizes the thumbprint authorizing use, 2) the connected mobile device provides the authorization for use, 3) programmed timing duration has passed. An illustrative example of the mobile device 2800 unlocking the pen injector cap 2808 from the pen injector body 2804 by recognizing an authorized thumbprint or other suitable authentication mechanism (e.g., password unlock), optionally resulting in the status indicator changing colors to reflect readiness to inject, is shown in FIG. 28. This additional level of tamper resistance helps ensure that only the authorized user can have access to the medication even in the circumstances that they attempt to remove the medication through the prefilled preloaded pen injector septum with a syringe. In some embodiments, to provide additional diversion or tamper resistance, the pen cap must be replaced after each injection. In such embodiments, if the cap is not replaced, the pen would be locked out and not provide any additional injections. When the cap is replaced properly, then the pen relocks the cap limiting access to the septum until another authorization is provided to again unlock the cap.

In another embodiment, the pen injector could use an electronically driven gearmotor and drive train coupled to a leadscrew that enables delivery of any amount from zero to the full deliverable volume within the reservoir. The electronically controlled gearmotor allows for software controlled delivery based on the programmed lockout timing and authorization within the electronic circuit. FIG. 26 shows a two-part prefilled and preloaded patient adjustable (dial-a-dose) pen injector having an electronically controlled gearmotor drive system with the activation button and dial-a-dose knob 2606, the reusable pen injector driver 2604, a fingerprint unlock pad 2608, a time controlled injection and cap lockout mechanism 2610, the pen injector plunger driver 2602, and the pen injector body assembly 2600. In this embodiment, a rheostat 2620 is incorporated to indicate to the electronic circuit the amount of dose set to be delivered and displayed to the user as shown in FIG. 26B. In some embodiments, the electronic circuit is configured to track and monitor one or more parameters of bolus delivery such as the number of boluses, their timing, the drug product volume and/or milligrams of drug product delivered and compare to the prescribed and/or preprogrammed delivery volumes authorized to be delivered. In the case that the user attempts to dial a dose that exceeds the authorized amount, the user interface on the pen injector would indicate that the allowable amount in the dose set window or indicate that the pen injector is not ready to inject as shown in FIG. 26B. As shown in FIG. 26A, the pen injector can include a rheostat to monitor dose dialed in for delivery 2620, an electronic circuit (can include crystal oscillator and battery) 2622, a gearmotor and leadscrew drivetrain assembly 2624 with the gearmotor and solenoid to disengage cap lockout pin 2626, and the cap lockout pin engaged with the locking cap on pen injector 2628. Also shown is the leadscrew driver from the gearmotor 2618, the leadscrew 2616, the cartridge plunger 2614, and the drug filled cartridge or reservoir 2612. FIG. 26B shows the activation button and dial-a-dose knob 2606 with dose set window 2632, readiness LED 2630 and fingerprint unlock pad 2608 that can optionally communicate with a mobile device 2636 using a wireless connection 2634.

FIG. 27 shows an example of fingerprint authorization for a single part prefilled and preloaded patient fixed dose pen injector or autoinjector with the pen injector body 2700 having a fingerprint scanner 2702 and coupled to the pen injector cap 2704.

### Pen Injector Septum Protection Configuration

Prefilled and preloaded commercial pen injectors or autoinjectors provide the ability to deliver the contents within the device based on the ability to activate and deliver from the device, the dose delivery settings, and the frequency of deliveries possible. For example, in a single bolus dose autoinjector, following removal of the protective cap, the total deliverable volume within the autoinjector will be administered following device activation. In some cases, activation occurs when the activation button is depressed while the needle shield is depressed against the patient's body unlocking the activation button. In some cases, only depressing the needle shield against the patient's body is needed to activate delivery. For pen injectors with one or multiple dose capability, the protective cap must first be removed, sterile needle assembly placed on the pen injector, the intended dose dialed in, the pen injector placed on the patient's body, then the activation button depressed to cause delivery. In both cases, drug delivery into the patient requires the protective cap to be removed from the autoinjector or pen injector body to allow access to the drug or the ability to deliver within the patient's body. Note that for autoinjectors that utilize a staked needle prefilled and preloaded syringe, once the protective cap is removed, the drug contained within the syringe can be withdrawn by sucking the medication out from the syringe needle using a tube applied around the needle and vacuum to withdrawal the drug, or by using a septum based cartridge assembly with a plunger rod to penetrate into the needle and withdrawal the drug.

In order to inhibit access to high value or controlled substance contained within the pen injector or autoinjector, the protective cap can be locked in place at time of or following device manufacture and only unlocked when an authorized unlocking signal is received by the pen injector or autoinjector by another device such as a mobile phone. FIG. 28 depicts a mobile interface to the pen injector or autoinjector that is necessary to unlock the respective protective cap and allow it to be removed from the pen or autoinjector body. Connectivity between the mobile device and pen injector or autoinjector could utilize multiple technologies commonly used such as but not limited to BlueTooth, WiFi, ZigBee, Near Field Communication (NFC), or electromagnetic induction. For example, it is common to connect a secondary device to a mobile phone using low power Bluetooth. Once the two devices are paired, the mobile interface can interrogate the pen injector or autoinjector, obtaining a multitude of information such as medicinal content, expiration date, lot number, manufacture, temperature within the pen injector or autoinjector, number of times the protective cap has been removed, amount of drug delivered, patient information, and the like. Additionally, the mobile device can send information to the pen injector including programming information such as bolus dose amounts and timing between boluses, fingerprint information for pen injectors or autoinjectors that have a fingerprint scanner functionality, and commands to unlock the pen injector or autoinjector or the like. In some embodiments, the mobile device must receive authorization to program the pen injector or autoinjector for any function or capability from an external service provider. The external service provider could require authorization from a healthcare provider, payer, or other party. The example in FIG. 28 shows a status indicator 2806 that can be green representing that the protective cap is unlocked and can be removed or a red status indicator representing the protective cap is locked and cannot be removed.

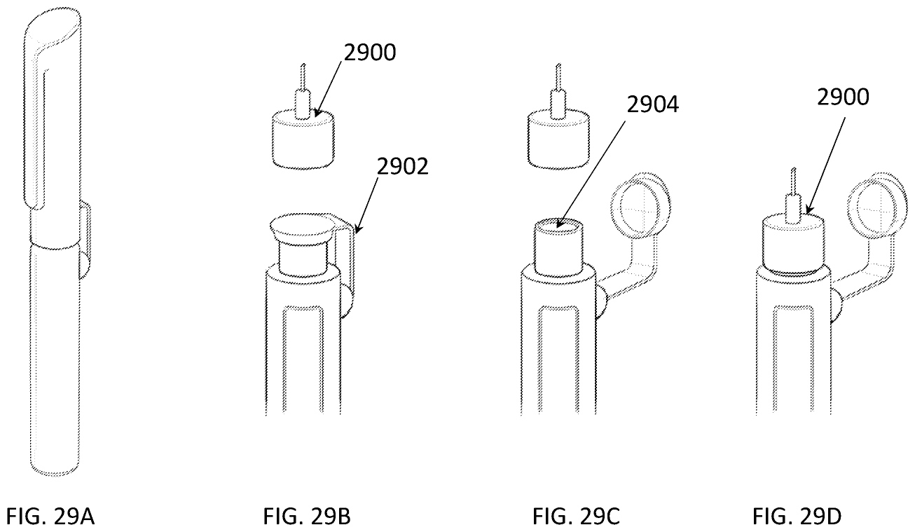

FIG. 29 depicts a cartridge septum lockout feature 2902 to inhibit unintended access to the cartridge septum. Having access to the cartridge septum could allow for unauthorized or unintended withdrawal of medication by using a syringe to puncture the cartridge septum and withdrawal the medication from the prefilled cartridge within the pen injector. In some embodiments, a programmable and software controlled lockout cap is incorporated within the pen injector that inhibits access to the cartridge septum until authorized by time delay between uses, mobile device command, fingerprint scan, combination lock, key, or other means. In some embodiments, the cartridge septum lockout utility is accomplished by implementing a septum lockout door or iris located in front of the cartridge septum within the pen injector to inhibit access to the cartridge septum, as shown in FIG. 29B. The cartridge septum lockout feature is unlocked following an authorized signal from a mobile device or based on internal control systems such as time delay between uses, fingerprint scan, combination lock, key, or other means. Access to the cartridge septum to enable drug delivery into the patient occurs as a single use disposable pen needle assembly 2900 is attached to the pen injector, as shown in FIG. 29B-29D. As shown, a cartridge septum lockout feature 2902 may be present that requires removal to allow the needle assembly 2900 to be attached to the pen injector on top of the cartridge septum 2904. In some embodiments, the cartridge septum lockout feature 2902 will automatically move from the unlocked state as shown in FIG. 29D to the locked state as shown in FIG. 29B to again inhibit access to the cartridge septum. In addition, in some embodiments, the pen injector cap could also be locked in pace following each placement on the pen injector body as described in FIG. 28.

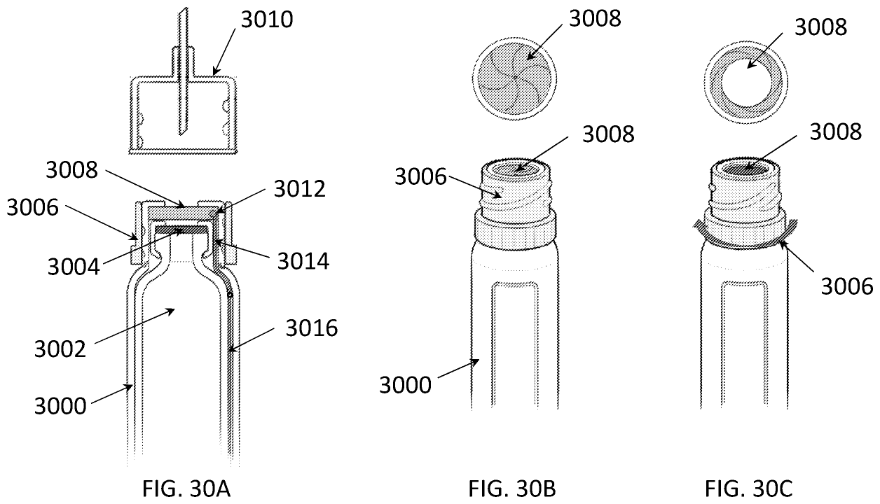

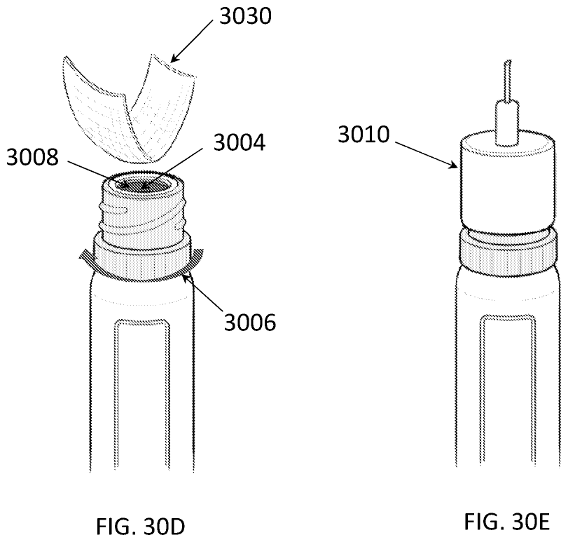

For high value or controlled substances, limiting access to the drug inhibits the potential for abuse and misuse. Controls could be implemented to lock the protective cap such that an authorized unlock signal is required to gain access to the medication. However, once the protective cap is unlocked and removed, there is potential access to the drug through the administration port. For example, on staked needle prefilled syringe systems, a tube could be placed over the syringe needle and the drug withdrawn by vacuum. For prefilled and preloaded cartridge based systems, an empty syringe could be used to withdraw the medication from the cartridge by penetrating the cartridge septum and withdrawing the medication. In order to inhibit access to the drug within a prefilled and preloaded cartridge, a featured could be implemented over the cartridge septum inhibiting access to the drug as shown in FIG. 30A-30B. In this state, the cartridge septum 3004 is blocked by an iris, inhibiting access to the cartridge septum to withdrawal the medication using a needle such as with a syringe. In order to unlock the iris lockout feature 3008, the iris activation body 3006 needs to be rotated as shown in FIG. 30C. However, the iris activation body 3006 is locked from rotation due to the iris activation lever 3012 interlock driven by the iris lockout flexible cable 3014 and lockout rod 3016 as shown in FIG. 30A. In some embodiments, the Iris lockout rod 3016 is controlled by the timer controlled injection and cap lock-out mechanism such as those shown in FIGS. 18-28. FIG. 30A-30B, show the iris lockout feature 3008 closed due to the locking action of the iris lockout rod 3016. FIG. 30A shows the needle assembly 3010, iris lockout feature 3008, iris activation lever 3012, iris activation body 3006, cartridge septum 3004, cartridge 3002, pen injector body 3000, iris lockout flexible cable 3014, and the iris lockout rod 3016. When the iris lockout rod 3016 is activated and pulled, thereby pulling the iris lockout flexible cable 3014, the iris activation body 3006 is unlocked, thereby allowing the iris activation body 3006 to be rotated, as shown in FIG. 30C. FIG. 30B shows the iris 3008 in the closed state in order to block access to the cartridge septum, and the iris activation body 3006, which is unable to rotate due to iris lockout rod engagement. FIG. 30C shows the iris 3008 in the open state allowing access to the cartridge septum with the iris activation body 3006 rotated to open the iris 3008. Once the Iris activation body 3006 is rotated, the cartridge septum 3004 is exposed, providing access to wipe the cartridge septum 3004 with a decontamination wipe 3030 to clean the cartridge septum 3004 as shown in FIG. 30D. FIG. 30D shows a decontamination wipe 3030 being used to wipe the accessible septum 3004 with the iris 3008 open following rotation of the iris activation body 3006 to open the iris 3008. FIG. 30E shows the patient needle assembly 3010 being placed on the pen injector allowing for medicinal delivery into the patient. In another embodiment, the iris activation feature could be activated by translation of the cartridge assembly within the pen injector body. The front portion of the cartridge assembly could press against the iris activation level resulting in opening the iris lockout feature along the needle assembly to be placed.

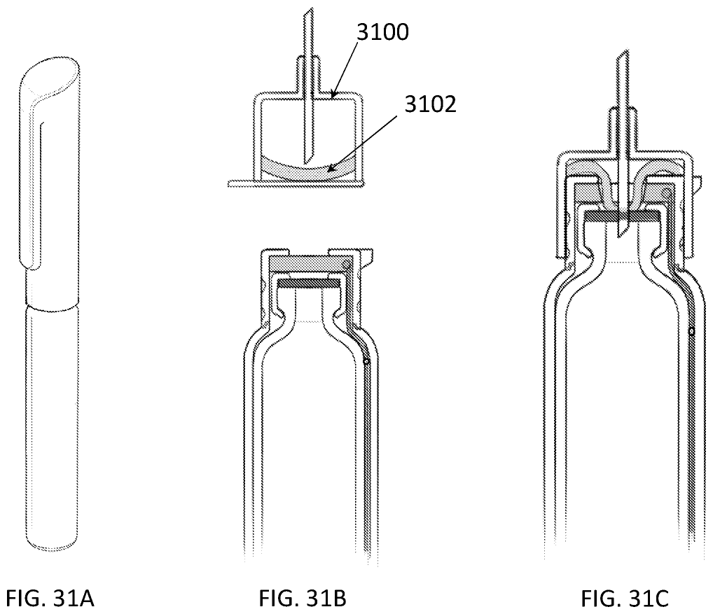

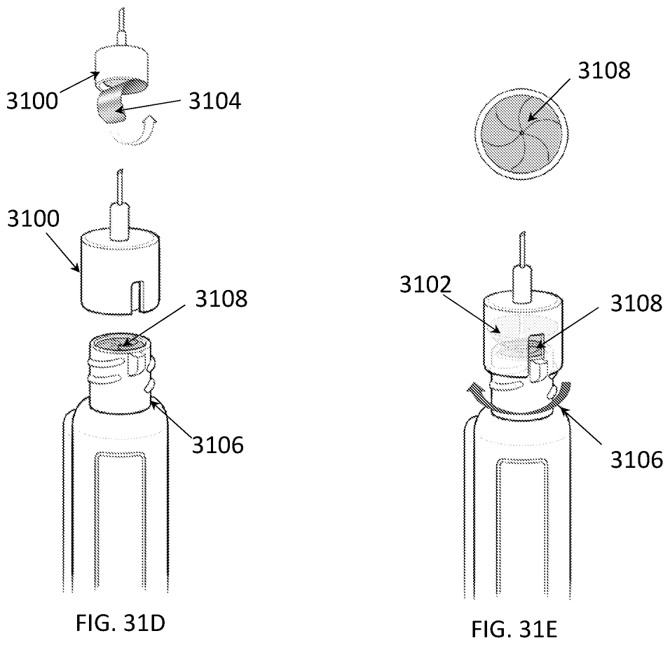

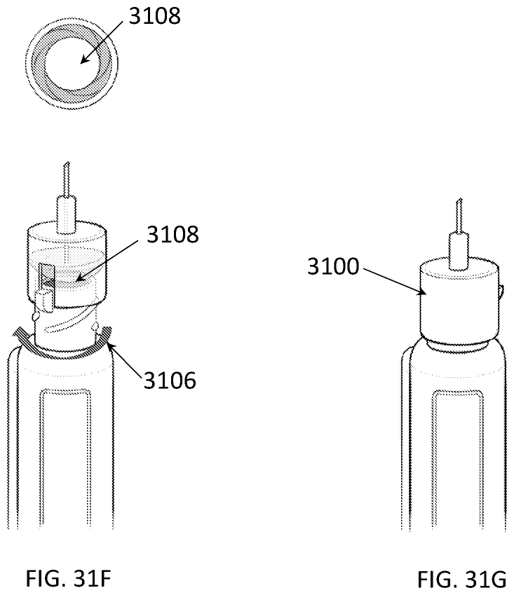

In another embodiment, a custom patient needle assembly, as shown in FIG. 31, is provided that incorporates a decontamination sponge for the purpose of decontaminating the cartridge septum when the custom needle assembly is placed onto the pen injector. Shown are the needle assembly 3100 with decontamination sponge 3102, with the iris 3108 in a closed state and the iris activation body closed 3108 (FIGS. 31D-31E), and the open iris 3108 with the iris activation body 3106 rotated to open the iris (FIG. 31F). FIG. 31F shows the iris 3108 in an open state with the activation body 3106 being rotated to open the iris 3108. FIG. 31G shows the needle assembly 3100 installed onto the injector. In some embodiments, the pen injector incorporates the iris lockout feature as described in FIGS. 30A-30C and shown in FIG. 31B-31F. The iris is shown in the locked state in FIGS. 30A, 31B, and 31D due to the action of the iris lockout rod, and shown in the unlocked state in FIG. 31C and FIG. 31F, thereby allowing the custom needle assembly 3100 to be placed on the pen injector. When the iris lockout rod is activated by the time controlled injection and cap lock-out mechanism as shown in FIGS. 18-28, the iris activation body 3106 is unlocked and able to be rotated by the action of placing the custom needle assembly 3100 as shown in FIG. 31E and FIG. 31F. As the custom needle assembly with decontamination sponge is placed on the pen injector, the cartridge septum is automatically wiped to decontaminate the septum. FIG. 31C and FIG. 31G show the single use custom needle assembly 3100 installed on the pen injector readying it for medicinal delivery. After delivery, the single use custom needle assembly can be removed by the patient, and the iris can again be closed to a locked state by the lockout rod inhibiting access to the cartridge septum.

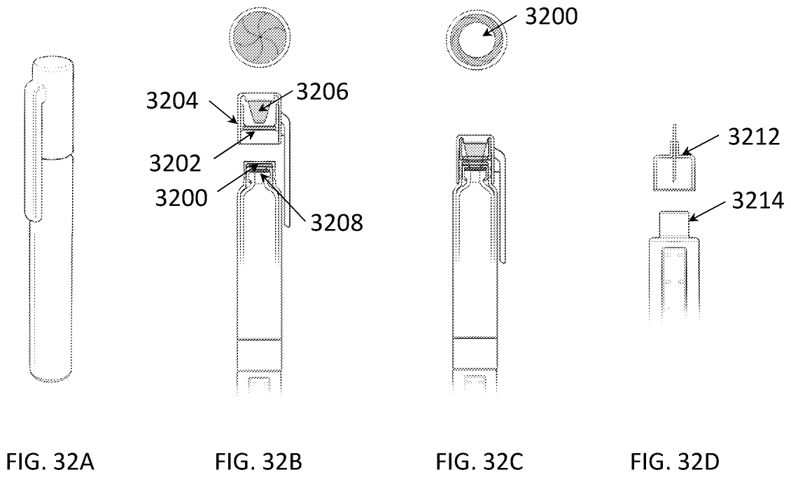

In another embodiment, the pen cap assembly incorporates the decontamination sponge with a pen cap iris to keep the decontamination sponge from drying out, as shown in FIG. 32B. The cartridge iris 3200 as shown in FIG. 32B is in the closed or locked state until the pen cap assembly 3204 is placed on the pen body. As the pen cap is placed on the pen body, both the pen cap iris 3202 and the cartridge iris 3200 are opened, providing access to the decontamination sponge 3206 to the cartridge septum 3208 thereby decontaminating the septum surface as shown in FIG. 32C. FIG. 32D shows the needle assembly 3212 in relation to the pen interface 3214 to the needle assembly. Any combination of lockout and needle assembly features could be utilized with this embodiment to inhibit unintended access to the medication within the pen injector or autoinjector.

In another embodiment, the lockout features described herein as applied to a cartridge system, could as well be applied to a luer or staked needle syringe or any type of medicinal reservoir container that has a means to deliver medication from the reservoir to a patient.

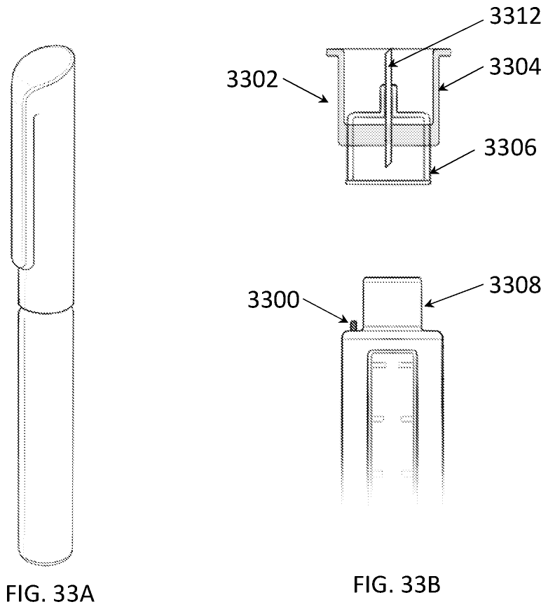

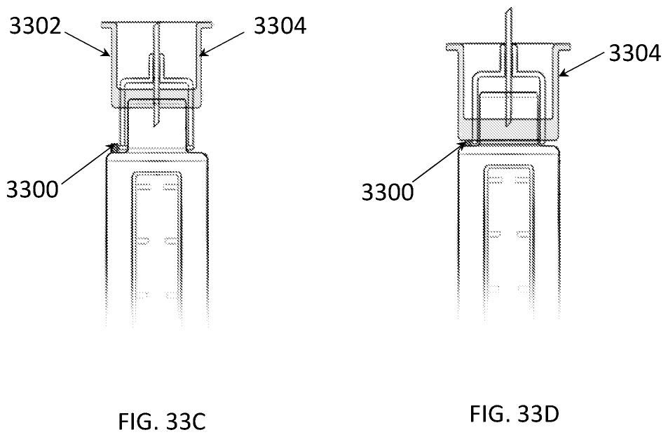

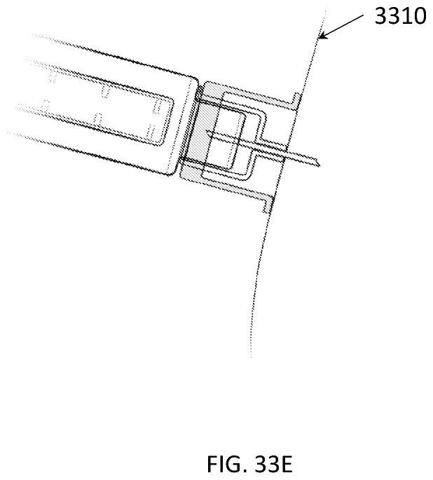

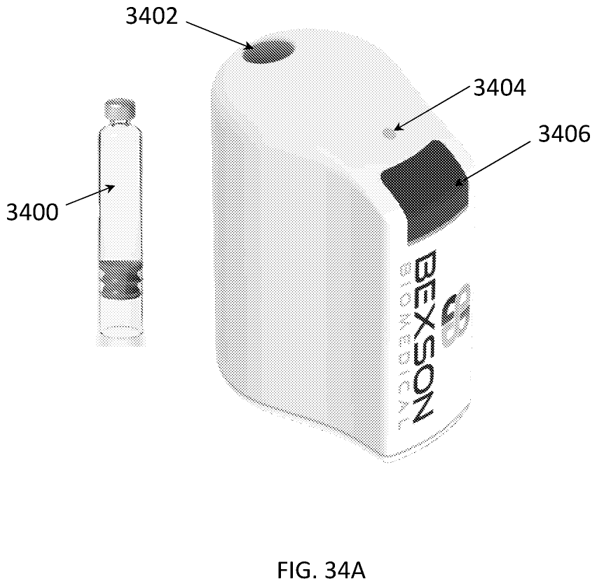

In another embodiment, the pen injector or autoinjector (FIG. 33A) incorporates a shield activated trigger as shown in FIG. 33B that, when depressed, unlocks the injection device enabling injection by the device button activation as shown in FIGS. 18-26. A shield activated needle assembly 3302 that incorporates a needle shield 3304, and that translates on the needle housing 3306 is used to activate the shield activation trigger 3300 as well as to limit viewability of the patient needle 3312 by the patient (FIG. 33B). The needle housing 3306 can interface with the injector via the interface 3308 to needle assembly. Obscuring the patient needle provides relief from the anxiety of seeing the needle penetrating the skin. FIG. 33C shows the shield activated needle assembly 3302 placed on a pen injector equipped with the shield activated trigger 3300 and the needle shield extended 3304; however, the needle shield has not yet been retracted in association with the attempt to deliver and therefore, the shield activated trigger is in the unactuated state. When the pen injector or autoinjector is placed and pressed against the body, the shield activated needle assembly is translated to the retracted state, thereby pressing and activating the shield activated trigger as shown in FIGS. 33D-33E enabling delivery. For delivery to occur, the device activation button such as the non-limiting embodiments shown in FIGS. 18-26 may be depressed.

In some embodiments, the pen injector or autoinjector is designed such that the device will activate, and delivery will occur when only the shield activated trigger is activated against the patient's body. This simplifies the use of the device since only one motion is required to initiate delivery. Therefore, in this embodiment, a device activation button as shown in FIGS. 18-26 is not needed.

### Auto-Injector Configuration

The device lockout features and controls as described herein apply to a fixed and dial-a-dose single or multidose pen injector and to a single bolus delivery autoinjector.

Waterproof System with Removable Cartridge

In some embodiments, the systems, devices, and methods disclosed herein provide a delivery device (e.g., a pump or pen injector) with a removable cartridge and cannula(s). In some embodiments, the delivery device is sealed, reusable, waterproof, or any combination thereof. In some embodiments, the cartridge when inserted aligns the plunger, the cannulas drive, the RFID reader and the magnetic sensors to enable an intrinsic relationship maintaining an easy to use device. In some embodiments, the device is configured with waterproof design. In some embodiments, the device utilizes a rechargeable battery and comprises a power management system configured to charge the battery utilizing a wireless power system such as an inductive charging system that uses electromagnetic induction to provide electricity to the battery.

### Cartridge Detection System

In some embodiments, the systems, devices, and methods disclosed herein provide a cartridge detection system. In some embodiments, the cartridge system utilizes a power harvesting near field communications system. In some embodiments, the harvested power is used to power an LED and sensor to enable level detection. In some embodiments, the system comprises a thin film resister with a wiper attached to the plunger to provide a resistance that is converted to a voltage to indicate plunger position. In some embodiments, the plunger uses a magnet and a Hall Effect device to show position. In some embodiments, the system comprises an RFID tag configured to provide data and sensor feedback and/or a unique pre-programmed code for cartridge security.

### Body Detection

In some embodiments, the systems, devices, and methods disclosed herein provide a body detection system using capacitive and/or resistive sensors to enable and track body contact. In some embodiments, the resistive sensors use simple spring loaded contacts that are pressed against the skin. In some embodiments, the sensors detect general skin resistance and water contact events for data analysis. In some embodiments, capacitive sensors are used to detect proximity to the body and obtain significant sensor reading changes when that proximity changes.

### Tamper Circuit and Sensors

In some embodiments, the systems, devices, and methods disclosed herein provide a cartridge comprising an RFID circuit that includes traces printed over the cartridge that, when broken, disable the device and indicate improper use through the data interface or cloud interface (e.g., via the user interface of a mobile device communicatively coupled to the device). In some embodiments, additional authentication is executed when doses are compared with plunger position over time. In some embodiments, the system generates an error when the user violates these parameters showing improper use or tampering.

### Multi-Layer Security System

In some embodiments, the systems, devices, and methods disclosed herein provide a multi-tier security system requiring the cartridge, the mobile device, the delivery device, or any combination thereof to report a security challenge response for each unique number relating to the cartridge, the device, the mobile device, or any combination thereof. In some embodiments, the registered devices and cartridges for a specific user are part of the security challenge. As part of the pairing, these numbers for that user and for these devices must be authenticated for the enable code. If security is breached the device and cartridge can be disabled from further use. This disable code and error cause is sent to the network for registration.

### Heart Rate Sensor

In some embodiments, the systems, devices, and methods disclosed herein provide a heart rate sensor configured to track heart rate over time and optionally proximity to the user for data related to the delivery system. The heart rate sensor can be an electrocardiography (ECG) sensor or photoplethysmography (PPG) sensor. Various algorithms can be used to process the sensor data to determine the heart rate, for example, the Pan-Tompkins algorithm.

Cannulas Insertion with Magnetic Position Sensor

In some embodiments, the systems, devices, and methods disclosed herein provide a cannulas system. In some embodiments, the cannula system comprises a magnetic element that can be monitored with a Hall Effect sensor. This enables a low drag simple system for determining cannulas position. In some embodiments, the magnetic element and sensor allow the spring loaded or the motor driven cannulas system to indicate insertion easily.

### Local Pain Button

In some embodiments, the systems, devices, and methods disclosed herein provide one or more buttons. In some embodiments, when a user taps a second capacitive button one to five times (or preset pattern), the system logs locally an acute pain event and shares that with the pain-tracking log. In some embodiments, pressing the button drives a dose directly if that option is available by prescription. In some embodiments, the device shares that information and log with the physician, e.g., over the cloud or network.

### Pain Tracking

In some embodiments, the systems, devices, and methods disclosed herein provide an app for tracking pain medication delivery. In some embodiments, the system tracks pain medication delivery through the device firmware and/or an app using the paired mobile device and data taken from the delivery device. In some embodiments, the delivery can be driven to a maximum medication dosage or maximum delivery of medication within a specified period authorized by the physician within a period of time through the ability for user requests for pain medication on demand. In some embodiments, as these pain reduction requests are made and at given intervals, the system or device enables push notifications that request pain ratings from the patient. This information can be used to drive delivery and feed information back to the prescribing doctor.

### Factory Sealed Drug Reservoir

In some embodiments, the systems, devices, and methods disclosed herein provide a drug reservoir. In some embodiments, the drug reservoir stores a such as ketamine. In some embodiments, the drug reservoir stores a drug that is a controlled substance. In some embodiments, the drug reservoir stores a drug that is susceptible to abuse. In certain embodiments, a proprietary formulation (e.g., containing ketamine) is loaded either in the factory or in the pharmacy, after which the fill port is be sealed off through any of variety of mechanisms including one or more of: a locking window, locking outer shell, rotation of the fill port away from the fill window, an outer shell sealed after drug reservoir is inserted, or other mechanical design.

Pre-filled Reservoirs: In certain embodiments, a proprietary ketamine formulation is provided to the patient in prefilled reservoirs.

Directional terms, such as “vertical,” “horizontal,” “top,” “bottom,” “upper,” “lower,” “inner,” “inwardly,” “outer” and “outwardly,” and “thinner” are used to assist in describing the present disclosure based on the orientation of the embodiments shown in the illustrations. The use of directional terms should not be interpreted to limit the invention to any specific orientation(s).

### Single-Component Devices

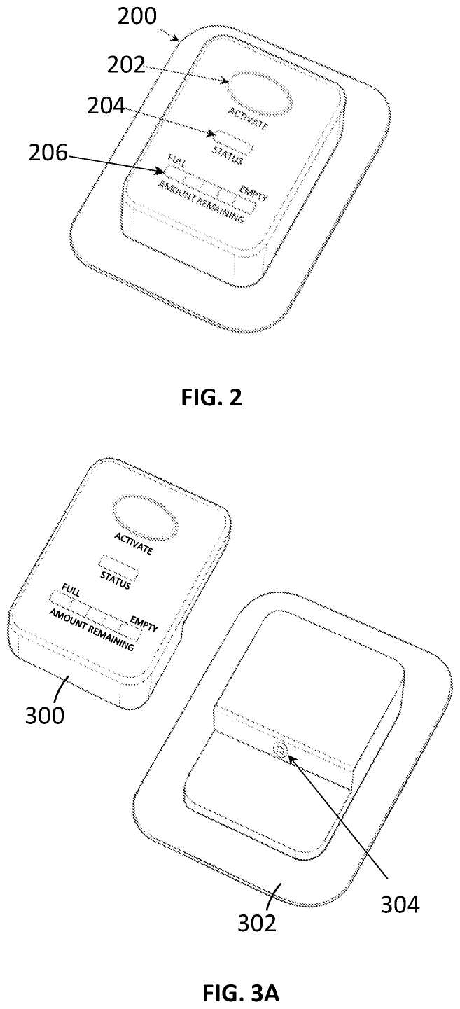

An exemplary embodiment of a drug delivery device provided herein is shown in FIG. 2. In this embodiment, the drug delivery device is a single use wearable patch pump with all aspects of the device configured as a single disposable component. The device comprises an adhesive patch 200 for application to the skin of a subject such that the subject can wear the device for the duration of the dosing schedule. Positioned on the adhesive patch opposite the adhesive surface is a compartment comprising the remaining components of the device, including a drug formulation containing reservoir, a pumping mechanism configured to pump the drug, an element such as a needle configured to deliver the drug formulation subcutaneously, a user interface, and any necessary or desired electronic components. The user interface is positioned atop the drug delivery device and is visible from the exterior. The remaining components are positioned on the interior of the device, which is configured to be tamper resistant to prevent access by the subject to the drug formulation. In this embodiment, the user interface comprises a button to activate the device 202, a pump status indicator light 204, and a light bar 206 showing the amount of drug formulation remaining (e.g., bolus indicator), which can also act as a leveling indicator.

The button to activate the device is configured to start a dosing regimen of the drug formulation according to a pre-programmed protocol which is not alterable by the subject. Once configured to start the dosing regimen, the device is configured to deliver a controlled, titratable amount of the drug formulation to the subject over a specified period of time, which is a period over several days, weeks, or months. In some embodiments, the activation button is also configured to allow delivery of bolus injections of the drug formulation as allowed by the pre-programmed protocol. For example, in embodiments where the drug formulation is a ketamine formulation for the treatment of pain, occasional bolus additions once every several hours may be allowed according to the pre-programmed protocol.

The user interface in this embodiment also comprises a pump status indicator. The pump status indicator is a multi-colored light system programmed to display a different color depending on the status of the pump. For example, the pump status indicator could be red when the pump is not operational, green when the pump is operational, and yellow when the pump is delivering a bolus addition, if allowed by the protocol. Additionally, the pump status indicator could be configured to convey additional information, such as when the pump is malfunctioning or when the drug reservoir is empty by emitting a flashing red light or similar signal. Alternatively, the pump status light could be configured to indicate whether or not an additional bolus of the medication is available to the subject, such as by displaying a green light when a bolus is allowed by the pre-programmed protocol and a red light when a bolus is not allowed by the pre-programmed protocol.

The user interface in this embodiment additionally comprises a light bar functioning as a remaining bolus and levelling indicator. During normal operation, the remaining bolus and levelling indicator is configured to display the amount of drug formulation left in the device as indicated by the proportion of lights which are activated. Additionally, the light bar is configured such that each light can be red, green, or yellow, and when the amount of drug formulation falls below a certain threshold the color of the light bar changes (e.g., when the drug formulation level drops below 40%, the light bar is yellow and when the drug formulation level drops below 20%, the light bar is red).

The light bar is also configured to operate as a levelling indicator. During initiation of the device and before it is applied to the subject, it may be desirable to prime the device and remove any air that may have been trapped in the drug formulation reservoir during manufacturing. In order to remove the trapped air from the device, it is necessary that the device be oriented in the proper position so the air can be expelled (e.g., with the top of the reservoir pointed in an upward direction such that air can be pumped out of the reservoir while retaining the drug formulation). Thus, during an initiation protocol, the light bar is configured to act as a levelling indicator for this priming step. During this priming step, the light bar is configured to display orientation information about the reservoir within the device.

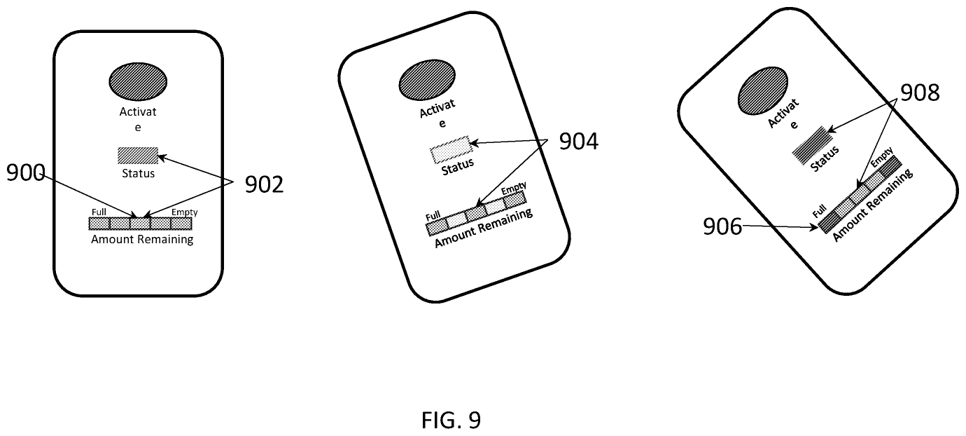

A non-limiting embodiment of one such configuration is shown in FIG. 9. In the example shown in FIG. 9, the light bar 900 is configured to display different combinations and colors of light in order to communicate orientation information to the subject. When the device is within a pre-determined threshold of the proper orientation (e.g., within a 5° angle of vertical orientation), the single middle light of the light bar shows a green signal. When the device is within a second pre-determined threshold of the proper orientation (e.g., from a 5° angle from vertical orientation to a 30° angle from vertical orientation), the two lights adjacent from the middle light of the light bar show a yellow signal. When the device is outside either of the pre-determined thresholds of the proper orientation (e.g., greater than a 30° angle from vertical orientation), the exterior lights of the light bar show a red signal. In this example, the orientation information is determined by a smart sensor (e.g., a Bosch Sensortec BNO055 smart sensor). Alternatively or in combination, the device can include a pump status indicator that communicates to a user whether the device is ready for priming/activation. For example, FIG. 9 shows the status indicator with a green light showing the device is ready to prime in the correct orientation 902, a yellow light indicating the orientation is incorrect but close to priming 904, and a red light indicating the orientation is off and needing rotation upwards to initiate priming 908.

The accelerometer of this smart sensor is configured to monitor for excessive movements during transportation or manufacturing of the device which may have caused any trapped air to break up into smaller bubbles. If acceleration or force has exceeded a pre-determined threshold prior to administration by the patient, the pump status light displays a red color during the priming step until sufficient time in the proper orientation has passed for the bubbles to coalesce, such sufficient time being pre-determined based on the acceleration measured. Once both the pump status light 902 and orientation lights 900 are green, indicating the system is prepared for priming, priming of the system begins automatically to purge the trapped air. Such a priming step is particularly important for sustained delivery of titratable drug formulations, as the presence of air in the system could affect the amount of drug actually administered.

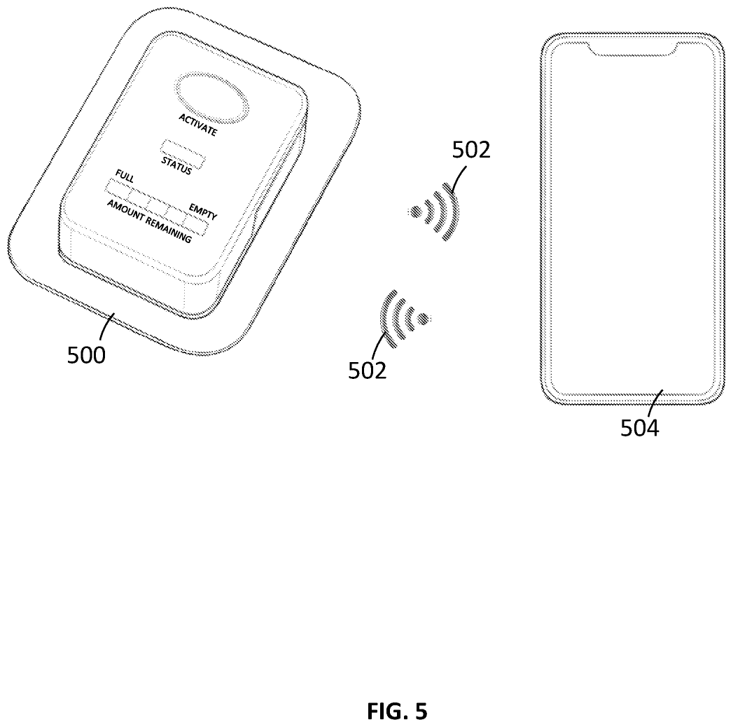

In some embodiments, the drug delivery device 500 is configured with a wireless communication mechanism such as Low-Power-Bluetooth which enables the device 500 to wirelessly connect 502 with a wireless enabled device 504, such as the subject cell phone, as indicated in FIG. 5. Through this connection, additional information about the device and formulation can be obtained by the subject, either through a standalone application or web-browser plug in, which acts as an extension of the user interface. The information available through the standalone application or web-browser plug in includes without limitation all of the information displayed on the user interface, the pre-programmed dosage regimen, certificate of analysis information of the formulation or device manufacturing, and/or expiration date of the device. Additionally, the wireless connection can enable real-time monitoring of the dosing by a medical professional, as well as the ability for the medical professional to modify the dosage regimen based on the medical professional's recommendation and patient needs. Preferably, the subject cannot modify the dosage regimen of his or her own accord.

### Two-Component Mixed Reusable and Disposable Devices

In another aspect, the disclosure herein provide a drug delivery device with a disposable component comprising a reservoir and a reusable component comprising a user interface, electronics (e.g., processor or computer system), power system, or any combination thereof. This configuration may enable a medical professional to prescribe a dosage regiment with multiple individually packaged dosage units that is more difficult for the subject to abuse by making it difficult to simultaneously administer drug from multiple devices. This configuration may also save costs due to the reusability of the user interface component across multiple reservoir components.

In another aspect, provided herein, is a drug delivery device comprising a) a user interface component comprising a user interface allowing a subject to self-administer a dose of a drug formulation, b) a reservoir component comprising a reservoir comprising the drug formulation, and c) a pump mechanism configured for administering the drug formulation, wherein the user interface and the reservoir belong to distinct components or portions of the overall device configured to be assembled by the subject. For example, the device can have a reusable component comprising the user interface, the electronics and power system, including a dock configured to engage with the disposable component comprising a reservoir containing the drug formulation. Accordingly, a user can insert or couple a disposable component such as a cartridge to the reusable component. The pump mechanism can be integrated with the user interface or the reservoir. In some embodiments, the user interface is configured to administer a pre-programmed dosage regimen. In some embodiments, the pre-programmed dosage regimen requires multiple reservoir components to be used sequentially. In some embodiments, the drug delivery device further comprises a system for expelling air from the drug delivery device.

In some embodiments, the reservoir component is a distinct component from the user interface component. In some embodiments, the reservoir component and the user interface component are separate pieces configured to be assembled by a user immediately before using the drug delivery device. In some embodiments, the two components are configured for assembly by a click mechanism, a snap mechanism, a screw mechanism, or any suitable mechanism.

In some embodiments, the reservoir component is disposable. In some embodiments, the reservoir component is configured for a single use.

In some embodiments, the reservoir component is tamper-proof. In some embodiments, the reservoir component is configured to not administer the drug formulation in the absence of the user interface component. In some embodiments, the reservoir is configured to be pierced by a needle on a separate component, such as the user interface component.

In some embodiments, the reservoir component further comprises the pump mechanism or a portion thereof, the system for expelling air or a portion thereof, a fluid path configured to deliver the dose of the drug formulation, or a component for attaching the device to the subject, or any combination thereof.

In some embodiments, the user interface component is reusable. In some embodiments, the user interface component is configured for use with multiple reservoir components in a sequential manner. In some embodiments, the user interface component is configured for use with a single reservoir component at one time. In some embodiments, the user interface component is configured not to accept multiple reservoir components at a single time. In some embodiments, the user interface component is configured to be used with at least 2, at least 3, at least 4, at least 5, at least 6, at least 7, at least 8, at least 9, or at least 10 individual reservoir components. In some embodiments, each individual reservoir component comprises an identical drug formulation.

In some embodiments, the user interface component further comprises electronics, a power system, the pump mechanism or a portion thereof, the system for expelling air or a portion thereof, or a component for attaching the device to the subject, or any combination thereof. In some embodiments, the user interface component further comprises electronics, a power system, or any combination thereof.

The user interface may comprise any of the embodiments described in the “Interfaces” section provided herein. In some embodiments, the user interface is physically on the user interface component. In some embodiments, the user interface is at least partially provided on a device in wireless communication with the user interface component. In some embodiments, the user interface is provided on a device in wireless communication with the user interface component.

In some embodiments, the user interface component is programmed or configured to operate with only a defined set of reservoir components. In some embodiments, the defined set of reservoir components comprises a specific set of reservoir components prescribed by a medical professional. In some embodiments, the specific set of reservoir components are prescribed as a single kit with the user interface component.

In some embodiments, the user interface component is configured to operate only with a prescribed number of reservoir components. In some embodiments, the user interface component is configured to operate with at most 2, at most 5, at most 10, at most 20, or at most 50 reservoir components.

In some embodiments, the reservoir component comprises an identification tag. In some embodiments, the reservoir component comprises an identification tag configured to be read by the user interface component. Any suitable identification tag may be employed. In some embodiments, the identification tag comprises a bar code, Near-Field-Communication (NFC) sensor in the disposable portion, and reader in the reusable portion, magnetic sensing such as Hall effect sensors, capacitive sensing, Radio-Frequency-Identification (RFID), or similar tag, or any combination thereof. In some embodiments, the identification tag is an RFID tag.

In some embodiments, the identification tag contains information about the pre-programmed dose regimen, the drug formulation, or the drug formulation component, or any combination thereof. Any relevant information can be stored on or associated with the identification tag. Such information can include medicine concentration and intended delivery parameters such as basal or bolus programming rates, amounts, and use duration. Other examples of such information include information on the medicine, date of manufacture, expiration date, manufacturing location, and the like.

In some embodiments, the user interface component comprises a reader configured to read the identification tag on the reservoir component. The type of reader on the user interface component will depend on the type of identification tag. In some embodiments, the reader is a sensor. In some embodiments, the sensor is a Radio Frequency Identification (RFID), or Near Field Communication (NFC) sensor. In some embodiments, the reader is configured to identify and operate with only a predefined set of reservoir components.

In some embodiments, the user interface component is configured for wireless communication with a wireless enabled device. In some embodiments, the user interface component comprises include Low-Power-Bluetooth (LPB), WiFi, or other telemetry protocols. In some embodiments, this allows for communication with an external device, such as a cellular device. In some embodiments, this enables the device to provide information on the use state of the wearable system or allow a medical professional to alter the pre-defined dosage regimen.

In some embodiments, the drug delivery device is configured for titrated delivery. In some embodiments, the drug delivery device is configured to deliver the drug formulation over a pre-determined period of time. In some embodiments, the drug delivery device is configured for intramuscular or subcutaneous administration of the drug formulation. In some embodiments, the drug delivery device is pre-filled and/or pre-loaded.

In addition to the features provided in this section, the drug delivery devices provided in this section may additionally comprise any of the properties, features, components, or other qualities provided in the “Drug delivery devices generally” section.

In some embodiments, the drug delivery devices provided herein are prescribed as a kit. In some embodiments, the kit comprises a single user interface component and a plurality of reservoir components. In some embodiments, the plurality of reservoir components comprises at least 2, at least 5, at least 10, or at least 50 reservoir components.

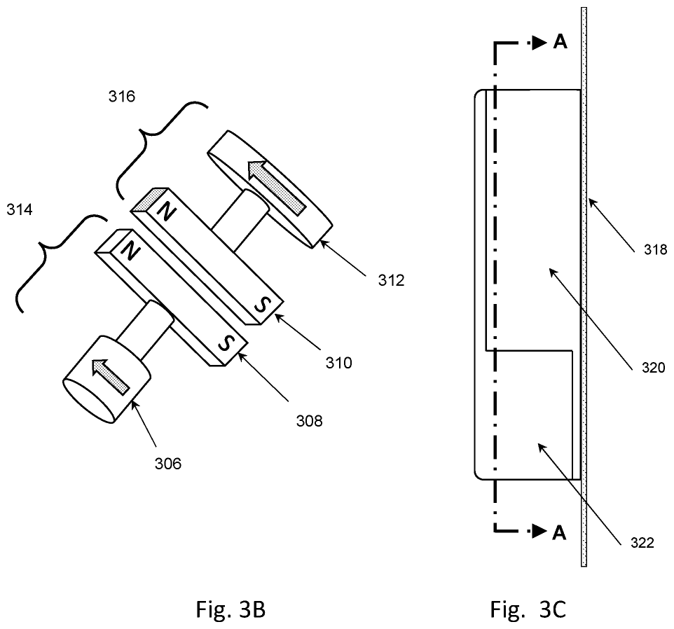

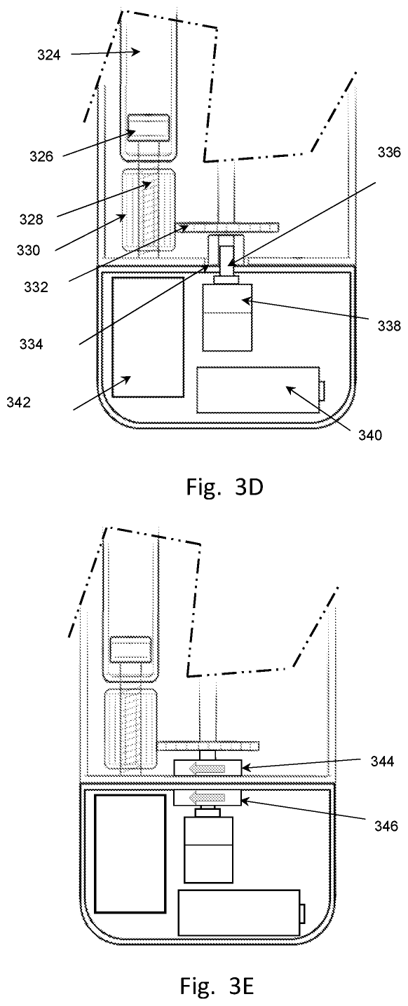

A further non-limiting embodiment of a device provided herein is shown in FIG. 3A, which shows a device comprising a reusable user interface component 300 comprising the electronic components of the device, drive gearmotor, and power systems and a disposable component 302 comprising a reservoir comprising a drug formulation, fluid path, and necessary drive components. The two distinct components are configured to be assembled by the subject in an easy to operate manner, such as by a simple clip mechanism. Only one disposable component can be assembled with the reusable user interface at a time. Once assembled, the device can have any of the components described with respect to the one or single part device and can include one or more of the following additional features. In some embodiments, the drive components within the disposable component 302 are driven by the power systems and gearmotor embodied within the reusable component 300 and transmitted via the drive coupling interface 304 as shown in FIGS. 3A-3E. The drive coupling interface 304 can be provided by mechanical interfacing features such as a square recess in the disposable component and a square protrusion from the reusable component, as shown in FIG. 3A and FIG. 3D. The shape of the mechanical interface could take many forms, including triangle, pentagon, hexagonal, heptagonal, star, Torx, or any other geometry that can provide torque or transmission between two members. FIG. 3C shows a side view of the device with the adhesive patch 318, disposable component 320, and reusable component 322 when assembled.

In some embodiments, the transmission coupling between the reusable component 300 and disposable component 302 could be magnetic utilizing one or more magnets on the reusable component, as shown in FIG. 3B and FIG. 3E. The magnetic coupling interface could utilize a combination of one or more magnets or ferrous magnetic plates on either or both of the reusable component 314 and disposable component 316 as shown in FIG. 3B. The magnetic coupling is designed such that rotary motion of the magnet or ferrous magnetic member 308, which is coupled to a driveshaft 306 driven by a gear motor within the reusable component, will result in a rotary motion of the magnet or ferrous magnetic member 310, which is coupled to a gear train 312 within the disposable component. A benefit of the magnetic coupling interface is that there are no external features visible on the disposable component enclosure or the reusable component enclosure that indicate the drive coupling interface. This provides for a clean look on the external encloser surfaces and ease of waterproofing the reusable and disposable components.

Accordingly, in some embodiments, the disposable component contains a complete drive system including an electric gearmotor that provides the transmission through a drivetrain that displaces the cartridge plunger providing fluid delivery through a patient administered fluid path to the patient. In the case where the gear motor is located within the disposable component, electrical contacts between the reusable component to the disposable component can be used to provide electrical power and drive control to the gear motor.

FIG. 3D shows a cross-sectional view of a non-limiting embodiment of the assembled device. The disposable component is shown with the primary medication container or reservoir 324 with the cartridge plunger 326, leadscrew 328, and drive nut 330, which is coupled to the drive wheel 332. The drive coupling nut 334 is also shown coupled to the drive coupling shaft 336. The reusable component is shown with the gear motor 338, battery 340, and electronic module 342. FIG. 3E shows the magnet 344 in the disposable component and the magnet 346 in the reusable component forming the magnetic coupling interface.

In some embodiments, the reusable user interface portion is configured such that it will only operate with a specified disposable component or a specified plurality of disposable components according to a pre-determined treatment protocol. The disposable component is designed to be tamper resistant and configured not to deliver the drug formulation in the absence of the user interface portion configured to operate with it.

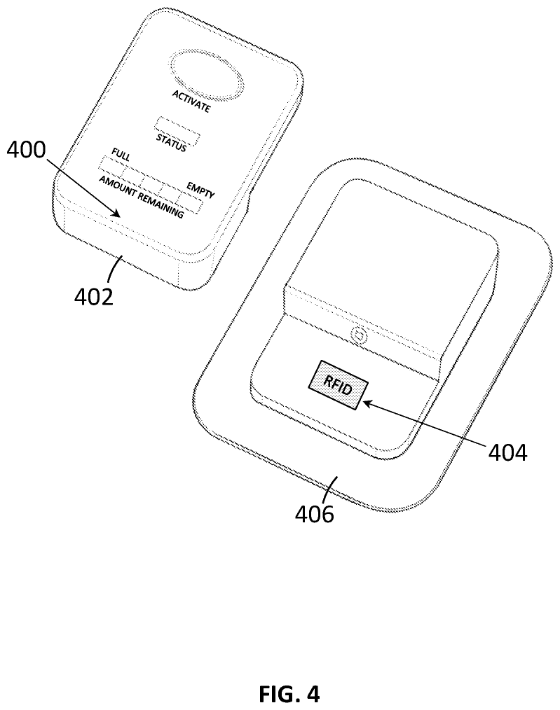

In some embodiments, the disposable component 402 comprises a radio-frequency-identification (RFID) tag 404 which can be read by an RFID reader 400 (not shown) positioned on the reusable user interface component 406 as indicated in FIG. 4. The reusable user interface component is configured such that when the RFID tag 404 is read, the device can determine if the disposable component is one of the specified plurality of disposable components to be used with the pre-programmed dosage regimen. The RFID interface also prevents unintended use or counterfeit use since the radio ID reader 400 can validate the information located on the RFID tag 404 for approved use.

The RFID tag also contains information on the drug concentration and intended delivery parameters such as basal or bolus programming rates, amounts, and use duration. Some medicines, such as ketamine, will benefit from having a factory preprogrammed set of basal rates and bolus amounts, so the tag in the disposable component will transmit the associated programming information to the reusable component when coupled together. The single reusable component is programmed by the disposable component as prescribed. The RFID tag located within or on the disposable component also includes information on the medicine, date of manufacture, expiration date, manufacturing location, and the like.

The devices described herein can be prescribed to a subject by a medical professional as a kit, wherein the kit contains a single re-usable component having a user interface and one or more disposable components containing the prescribed drug formulation (e.g., a plurality of disposable components such as 5-10 for a long dose regimen). The kit so configured minimizes the risk of a subject administering more of the prescribed drug formulation than prescribed because the subject is prescribed only a single delivery device. By contrast, if a subject is prescribed multiple doses of the prescribed drug formulation via integrated devices without a removal cartridge, there is a risk the subject can place multiple devices on their body simultaneously in order to exceed the prescribed dose of the drug. This is a special concern for controlled substance drug formulation that are prone to abuse, such as ketamine. Additionally, the tamper proof nature of the individual disposable components also prevents the foreseeable misuse of drug. Thus, the devices described herein provide distinct advantages to those of fully integrated single use devices.

### Three-Component Mixed Reusable and Disposable Devices

In another aspect, provided herein, is a drug delivery device comprising: a) a user interface component comprising a user interface allowing a subject to self-administer a dose of a drug formulation; b) a pump mechanism configured for administering the drug formulation; c) a reservoir component comprising a reservoir comprising the drug formulation; and d) a body contact surface component configured for attachment of the device to the subject's body, wherein the user interface, the reservoir, and the body contact surface are each part of distinct components configured to be assembled by the subject. The pump mechanism can be integrated with the user interface, the reservoir, or the body contact surface component.

In some embodiments, the reservoir component, the body contact surface component, and the user interface component are each distinct components. In some embodiments, the reservoir component, the body contact surface component, and the user interface component are each separate components configured to be assembled by a subject immediately before using the drug delivery device. In some embodiments, the three components are configured for assembly by a click mechanism, a snap mechanism, a screw mechanism, or any suitable mechanism, or any combination thereof.

In some embodiments, the reservoir component is disposable. In some embodiments, the reservoir component is configured for a single use.

In some embodiments, the reservoir component is tamper-proof. In some embodiments, the reservoir component is configured to not administer the drug formulation in the absence of the user interface component, the body contact surface component, or both. In some embodiments, the reservoir is configured to be pierced by a needle on a separate component, such as the user interface component or the body contact surface component. In some embodiments, the reservoir component is not substantially tamper-proof.

In some embodiments, the reservoir component further comprises the pump mechanism or a portion thereof, the system for expelling air or a portion thereof, or a fluid path configured to deliver the dose of the drug formulation, or any combination thereof. In some embodiments, the reservoir component consists essentially of the reservoir and a suitable reservoir cap.

In some embodiments, the body contact surface component is disposable. In some embodiments, the body contact surface component is configured for a single use.

In some embodiments, the body contact surface component further comprises the pump mechanism or a portion thereof, a system for expelling air or a portion thereof, or a fluid path configured to deliver the dose of the drug formulation, or any combination thereof.

In some embodiments, the body contact surface component is sterilized separately from the other components prior to assembly. In some embodiments, the body contact surface component is stored in a sterilized blister pack prior to assembly. In some embodiments, the blister pack is sterilized by ethylene oxide treatment.

In some embodiments, the user interface component is reusable. In some embodiments, the user interface component is configured for use with multiple reservoir components in a sequential manner. In some embodiments, the user interface component is configured for use with a single reservoir component at one time. In some embodiments, the user interface component is configured not to accept multiple reservoir components at a single time. In some embodiments, the user interface component is configured to be used with at least 2, at least 3, at least 4, at least 5, at least 6, at least 7, at least 8, at least 9, or at least 10 individual reservoir components. In some embodiments, each individual reservoir component comprises an identical drug formulation.

In some embodiments, the user interface component is configured for use with multiple body contact surface components in a sequential manner. In some embodiments, the user interface component is configured for use with a single body contact surface component at one time. In some embodiments, the user interface component is configured not to accept multiple reservoir components at a single time. In some embodiments, the user interface component is configured to be used with at least 2, at least 3, at least 4, at least 5, at least 6, at least 7, at least 8, at least 9, or at least 10 individual reservoir components.

In some embodiments, the user interface component further comprises electronics, a power system, the pump mechanism or a portion thereof, or a system for expelling air or a portion thereof, or any combination thereof. In some embodiments, the user interface component further comprises electronics, a power system, or any combination thereof.

The user interface may comprise any of the embodiments described in the “Interfaces” section provided herein. In some embodiments, the user interface is physically connected to the user interface component. In some embodiments, the user interface is at least partially provided on a device in wireless communication with the user interface component. In some embodiments, the user interface is provided on a device in wireless communication with the user interface component.

In some embodiments, the user interface component is programmed or configured to operate with only a defined set of reservoir components. In some embodiments, the defined set of reservoir components comprises a specific set of reservoir components prescribed by a medical professional. In some embodiments, the specific set of reservoir components are prescribed as a single kit with the user interface component.

In some embodiments, the user interface component is configured to operate only with a prescribed number of reservoir components. In some embodiments, the user interface component is configured to operate with at most 2, at most 5, at most 10, at most 20, or at most 50 reservoir components.

In some embodiments, the reservoir component comprises an identification tag. In some embodiments, the reservoir component comprises an identification tag configured to be read by the user interface component. Any suitable identification tag may be employed. In some embodiments, the identification tag comprises a bar code, Near-Field-Communication (NFC) sensor in the disposable component, and reader in the reusable component, magnetic sensing such as Hall effect sensors, capacitive sensing, Radio-Frequency-Identification (RFID), or similar tag, or any combination thereof. In some embodiments, the identification tag is an RFID tag.

In some embodiments, the identification tag contains information about the pre-programmed dose regimen, the drug formulation, or the drug formulation component, or any combination thereof. Any relevant information can be stored on or associated with the identification tag. Such information can include medicine concentration and intended delivery parameters such as basal or bolus programming rates, amounts, and use duration. Other examples of such information include information on the medicine, date of manufacture, expiration date, manufacturing location, and the like.

In some embodiments, the user interface component comprises a reader configured to read the identification tag on the reservoir component. The type of reader on the user interface component will depend on the type of identification tag. In some embodiments, the reader is a sensor. In some embodiments, the sensor is a Radio Frequency Identification (RFID), or Near Field Communication (NFC) sensor. In some embodiments, the reader is configured to identify and operate with only a predefined set of reservoir components.

In some embodiments, the user interface component is configured for wireless communication with a wireless enabled device. In some embodiments, the user interface component comprises include Low-Power-Bluetooth (LPB), WiFi, or other telemetry protocols. In some embodiments, this allows for communication with an external device, such as a cellular device. In some embodiments, this enables to device to provide information on the use state of the wearable system or allow a medical professional to alter the pre-defined dosage regimen.

In some embodiments, the drug delivery device is configured for titrated delivery. In some embodiments, the drug delivery device is configured to deliver the drug formulation over a pre-determined period of time. In some embodiments, the drug delivery device is configured for intramuscular or subcutaneous administration of the drug formulation. In some embodiments, the drug delivery device is pre-filled and/or pre-loaded.

In addition to the features provided in this section, the drug delivery devices provided in this section may additionally comprise any of the properties, features, components, or other qualities provided in the “Drug delivery devices generally” section.

In some embodiments, the drug delivery devices provided herein are prescribed as a kit. In some embodiments, the kit comprises a single user interface component, a plurality of reservoir components, and a plurality of body contact surface components. In some embodiments, the plurality of reservoir components are stored in a tamper resistant package configured to dispense a subset of the reservoir components according to a pre-programmed dosage regimen. The tamper resistant package may be any of the tamper resistant packages provided in the “Tamper Resistant Packaging” section. In some embodiments, the kit further comprises a sterilization agent, such as an alcohol wipe. The sterilization agent can be used to sterilize the surface of a reservoir component prior to assembly of the drug delivery device.

In some embodiments, the plurality of reservoir components comprises at least 2, at least 5, at least 10, or at least 50 reservoir components. In some embodiments, the plurality of body contact surface components comprises at least 2, at least 5, at least 10, or at least 50 body contact surface components.

A non-limiting embodiment of a device provided herein is a device which comprises two distinct disposable components and a single reusable component. The three distinct components are configured to be assembled by a subject in an easy to operate manner, such as by simple clipping mechanisms. The assembled device can include any of the internal or external features described with respect to the single or two part devices. The single reusable component can be substantially similar to the user interface component described for single and/or two part devices. However, rather than having a single disposable component comprising a reservoir comprising a drug formulation, fluid path, and necessary drive components, the reservoir comprising the drug formulation is separate from the fluid path and drive components until assembled by the subject.

In some embodiments, the first disposable component comprises the drug reservoir containing the drug formulation. This component is designed such that the interior of the reservoir containing the drug formulation is sterilized at the time of manufacturing using fill and finish techniques. The drug formulation is sealed in the reservoir with a cartridge septum which can be readily pierced using a needle from the second disposable component.

In some embodiments, the second disposable component comprises the fluid path and necessary drive components to operate the device, as well as the adhesive surface configured to attach the device to the subject. This second disposable component also contains a needle configured to pierce the septum of the drug reservoir, thereby creating a fluid connection between the flow path and the drug formulation. The second disposable component is assembled by the manufacturer and packaged in a blister package, which is then sterilized by ethylene oxide gas.

In some embodiments, the second disposable component is configured such that once the first disposable component is placed within the second disposable component, it is locked or latched in place and not removable. This ensures that when the two disposable components are coupled, access to the reservoir or cartridge septum and therefore medication container within is limited, providing additional abuse or misuse protection.

One benefit of this two disposable component design is the ability to sterilize and package one or more disposable components separately from the reusable component thereby providing the opportunity for a better user interface on the reusable component. The ability to do independent sterilizations of the liquid formulation containing reservoir and the flow path components greatly eases manufacturability of the final device. Additionally, separating the prefilled container and the wearable contacting disposable significantly simplifies the design, manufacturing, and storage processes required to maintain sterility over the storage and use periods of these sub-systems.

However, the benefits to manufacturability of the two disposable component device led to the reservoir component having less inherent tamer proof capabilities, as the septum cap could be pierced by a needle and a syringe used to withdraw the medication through the septum and used against prescription by the subject. To address this risk, the device can be provided as a kit comprising the reusable user interface component, for example, a blister pack containing multiple second components comprising the fluid path and adhesive surface for attaching the device to the subject, and a tamper resistant (TR) package which contains the drug formulation filled reservoir components.

In some embodiments, the TR Package is designed to contain one or more prefilled containers, is structurally sound to inhibit access, and is locked. The TR package can include internal electronics that are programmed at the time of pharmacy distribution, and include information on the drug product contents, expiry date, MFG date, dose information, pump programming settings, and the like. In some embodiments, the electronic systems within the TR Package manage the locking system such that the lock is only unlocked by authorized users such as the patient. For example, a key to unlock the TR Package is coupled through the user interface component of the kit, which are configured to communicate through Low-Power Blue Tooth. To unlock the TR Package, encoded information within the user interface component and the TR Package is compared and authenticated. If properly authenticated and at an acceptable time according to the pre-programmed dosage regimen, the TR package unlocks and dispense a single reservoir containing the drug formulation. The subject then sterilizes the surface of the reservoir component (such as with an alcohol wipe), assembles or reassembles the full device, and begins or continues to administer according to the pre-programmed dosage regimen.

### Tamper Resistant Packaging

In another aspect, a tamper resistant package for storing reservoir components comprising drug formulations is provided herein. The tamper resistant package is useful for preventing access to the reservoir components by a subject outside of a pre-determined dosage regimen. By preventing access to the reservoir components, abuse of the drug formulation stored therein can be prevent and compliance with the pre-determined dosage regimen can be maintained.

In another aspect, provided herein, is a tamper resistant package configured to dispense a subset of reservoir components according to a pre-programmed dosage regimen. In some embodiments, the tamper resistant package provided herein is a part of a kit for assembling any of the devices provided herein. In some embodiments, the tamper resistant package is a part of a kit for assembling a drug delivery device provided in the “Two-Component Mixed Reusable and Disposable Devices” section. In some embodiments, the tamper resistant package is a part of a kit for assembling a drug delivery device provided in the “Three-Component Mixed Reusable and Disposable Devices” section.

In some embodiments, the tamper resistant package is built to be structurally sound. The structural soundness of the tamper resistant package prevents access to the reservoir components disposed within the tamper resistant package until a prescribed or pre-determined time of intended use of the reservoir component. The material used to make the tamper resistant package should be sufficiently strong to prevent easy access to the contents of the tamper resistant package outside the intended time to dispense the reservoir components disposed therein. Such materials can include thick plastics, metals, or other material of a suitable hardness. In some embodiments, the walls of the tamper resistant package comprise an exterior of plastic with an addition material disposed within, such as a metal. The tamper resistant package may also comprise an additional material disposed within the package to prevent breakage of the reservoir components during shipping and handling.

In some embodiments, the tamper resistant package comprises a locking mechanism. Any suitable locking mechanism can be used, including latches, deadbolts, padlocks, and the like. In some embodiments, the locking mechanism is configured to remain locked until an indicated time or condition according to a pre-determined dosage regimen. For example, a cartridge may comprise a locking mechanism that prevents access to the drug formulation in its reservoir until receiving an activation signal from the drug delivery device (e.g., in the 2- or 3-part configurations). The drug delivery device can be configured to send the activation signal only after authenticating the cartridge (e.g., based on RFID information from the cartridge indicating the appropriate dosage regimen and/or remaining/updated dosage information after previous use).

In some embodiments, the tamper resistant package is configured at the time of manufacture, prescription, or pharmacy distribution to open or dispense a reservoir component at an indicated time or condition.

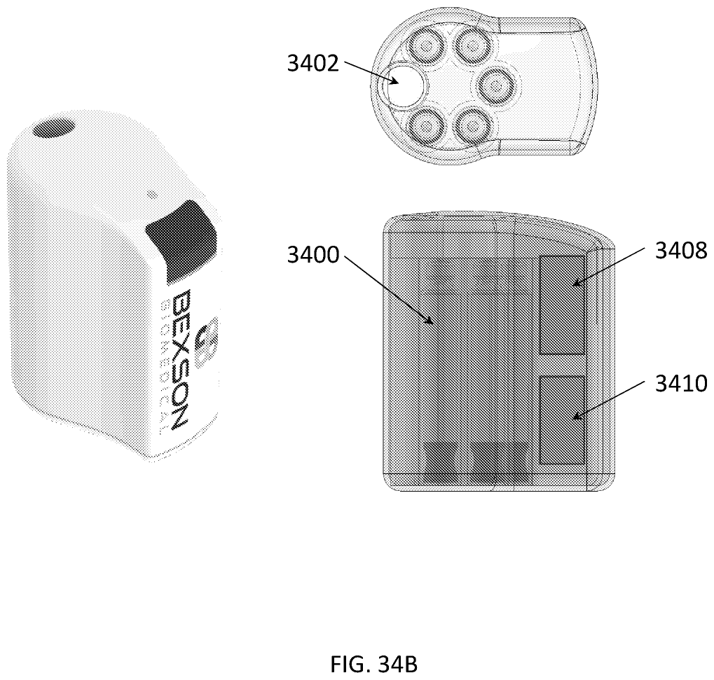

In some embodiments, the tamper resistant package is configured to open, unlock, or dispense a reservoir component after a pre-selected amount of time. Any preselected amount of time may be used in order to facilitate the pre-determined dosage regimen. The preselected amount of time may be any number of hours, such as 4 hours, 8 hours, 12 hours, 24 hours, or 36 hours. The preselected amount of time may be any number of days, such as one day, two days, three days, four days, five days, six days, or seven days. The preselected amount of time be repeated or varied throughout the pre-determined dosage regimen, such as opening, unlocking, or dispensing a reservoir component at periodic or variable intervals. The tamper resistant package may also be configured to open, unlock, or dispense a reservoir component at specified dates or times of day according to the pre-determined dosage regimen. FIG. 34A shows an example of a tamper resistant package (e.g., a tamper resistant dispenser) designed to release one or more prefilled cartridges 3400 after an authorizing signal is received by the internal control system. Also shown is the cartridge access port 3402, status indicator 3404, and activation button 3406. With each authorization signal, the next cartridge within the tamper resistant package will be released, and the remaining cartridges will remain locked within until the next authorization signal is received. The tamper resistant package can contain one or multiple reservoir components. FIG. 34B shows an example of a tamper resistant package designed to release five cartridges 3400 in sequential order from a cartridge access port 3402 after an authorization signal is received to release a single cartridge. The package can include an electronic module 3408 to track the number of cartridges dispensed and the drive system 3410 to index the internal cartridges to enable access as shown in FIG. 34B.

In some embodiments, the tamper resistant package comprises a plurality of locking mechanisms, each locking mechanism keeping a subset of the plurality of reservoir components locked separately. In such a configuration, each locking mechanism may be independently configured to open at or after a specified time to allow access to the subset of reservoir components. The subset can be any number of reservoir components, but is preferably a single reservoir component. In some embodiments, the subset comprises at most 1, at most 2, or at most 3 reservoir components.

In some embodiments, the locking mechanism is configured to be unlocked in response to a signal from an authorized user, such as a subject or prescribing medical professional. In some embodiments, the locking mechanism is unlocked by a key through an electronic device of the authorized user, such as a user interface component provided herein or a mobile device. In some embodiments, the locking mechanism is in wireless communication with the electronic device. In some embodiments, the key of the electronic device is determined and programmed according to the pre-determined dosage regimen.

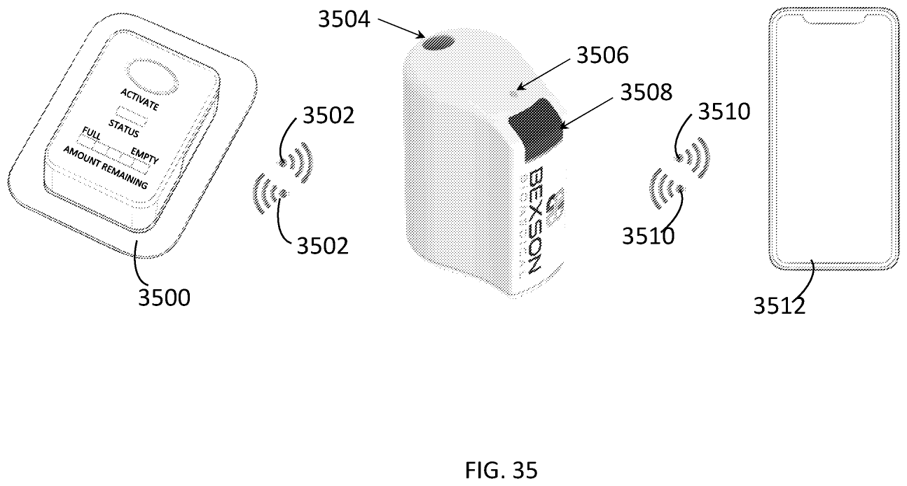

In some embodiments, the tamper resistant package is enabled for wireless communication, such as through RFID, Low Power BlueTooth (LPBT), Near Field Communication (NFC), Zigby, bar code, or other available communication protocols. In some embodiments, the tamper resistant package unlocks after encoded information within the transmitting device (mobile phone or user interface portion of the drug delivery device) and the tamper resistant package is compared and authenticated. In some embodiments, the tamper resistant package is configured to provide real time data to a third party, such as a medical professional, to ensure the pre-determined dosage regimen is being followed by the subject. FIG. 35 shows an example of a tamper resistant package designed to release a reservoir component after an authorizing signal is received by either the wearable delivery system or by a mobile phone. The wearable delivery system 3500 can communicate wirelessly 3502 with the tamper resistant cartridge dispenser which may have a cartridge access port 3504, status indicator 3506, and activation button 3508. The cartridge dispenser may also communicate wirelessly 3510 with a mobile device 3512. Note that any mention of wireless communications shall be interpreted as also disclosing an alternative wired communication mechanism between the various systems, dispensers, devices, pumps, and injectors disclosed herein.

In some embodiments, the tamper resistant package comprises internal electronics. In some embodiments, the internal electronics comprise an internal power source, such as a battery. In some embodiments, the internal electronics are powered by an external power source, such as a magnetic, capacitive, inductive, or radiofrequency power source. In some embodiments, the internal electronics are configured to store information about the drug product contents, expiration date, manufacturing date, dose information, pump programming settings, or the like. In some embodiments, the internal electronics are programmed to unlock or open the device or dispense a reservoir component at a specified time or after a specified interval of time. In some embodiments, this information is stored in an active or passive memory within the tamper resistant package.

In some embodiments, the tamper resistant package comprises GPS or other track-and-trace sensors. In some embodiments, the GPS or track-and-trace sensors allow monitoring of distribution and supply chain information.

### Priming Systems

In another aspect, provided herein, is a system for priming a drug delivery device and removing air from a drug reservoir of the drug delivery device. Removal of air from the drug delivery device is especially important for controlled, titratable drug formulation delivery over time. The presence of air in the system can cause miscalculations of the amount of drug administered to a subject, thereby causing deviation from a desired or pre-determined dosage regimen. Additionally, many manufacturing processes for preparing pre-filled reservoirs for delivery of drugs result in air being trapped in the reservoir, particularly “plunger fill” processes for filling standard septum based cartridges that contain an elastomeric plunger. While there exist fill/finish processes for filling cartridges and reservoirs from the “neck” of the device that yield low bubble and near bubble-free fills, such processes are rare and difficult to implement. Thus, a method of priming a pre-filled reservoir contained in a drug delivery device at time of use would be highly beneficial in any drug delivery device intended to deliver titratable sustained drug delivery. The priming systems provided herein are compatible with any of the drug delivery devices provided herein, including fully integrated single-use drug delivery devices, two-component mixed reusable and disposable drug delivery devices, three-component mixed reusable and disposable drug delivery devices, or any other drug delivery device configured for administration by injection.

In another aspect, provided herein, is a drug delivery device comprising a) a pump mechanism configured for administering a drug formulation from a reservoir; and b) a user interface comprising an indicator configured to display orientation information of an outlet of the reservoir. In some embodiments, the pump mechanism is configured to expel air from the reservoir when the indicator displays that the outlet is oriented in an upward direction. The pump mechanism can be integrated into the component of the drug delivery device containing the reservoir. This integration can be part of a tamper resistant configuration in which only authorized or authenticated use of the drug delivery device with the cartridge allows the device to be unlocked and actuate the pump mechanism for drug delivery. By comparison, for example, separating the pump mechanism from the cartridge will require some port or outlet being available for the pump mechanism to extract the drug formulation from the reservoir in the cartridge. This port or outlet may pose a risk of user tampering, for example, inserting a syringe or needle through the port or outlet to extract the drug formulation. Although certain locking mechanisms may be utilized, the pump mechanism can be integrated into the cartridge to prevent or resist tampering.

In some embodiments, the outlet of the reservoir is not visible from a position exterior of the device. In some embodiments, the outlet of the reservoir is positioned on an interior of the device. In some embodiments, the outlet of the reservoir is the same as the flow path used for administration of the drug formulation to a subject. In some embodiments, the outlet of the reservoir is disposed on the flow path used for administration of the drug formulation to a subject. In some embodiments, the outlet of the reservoir is different from the flow path used for administration of the drug formulation to the patient. In some embodiments, expelling the air from the reservoir comprises driving the air through an injection needle.

In some embodiments, the user interface displays information relating to the orientation of the outlet, reservoir, cartridge, or device. The user interface can display this information via a graphical display on the device or simpler display element such as a light-bar and/or indicator light. In some cases, information relating to the orientation is transmitted to a mobile or wireless device such as a smartphone to be displayed or otherwise communicated to the user (e.g., audio command indicating delivery device is ready for priming or removal of air/gas from the drug reservoir).

In some embodiments, the pump mechanism is configured to automatically expel the air from the reservoir when the indicator displays that the outlet (or the reservoir, cartridge, or device) is oriented in the appropriate direction (e.g., upward direction).

In some embodiments, the user interface prompts a user to manually operate the pump to expel the air from the reservoir when the outlet is oriented in an upward direction, or provides an indication that the device is ready for removal of air (or any other gas) from the reservoir.

In some embodiments, the pump mechanism is configured to stop the pump once all of the air is expelled from the reservoir. In some embodiments, the pump mechanism comprises a torque or force sensor configured to automatically stop the pump mechanism when an increase in pressure is detected. In some embodiments, the pump mechanism comprises a torque or force sensor configured to detect an increase in pressure corresponding to successful completion of air removal from the reservoir. For example, the increase in pressure can indicate successful removal of air or gas since the liquid formulation will produce greater resistance to the pump mechanism. Accordingly, the user interface can be configured to display or otherwise provide an indication to the user that the air has been successfully removed. In some embodiments, the user can rely upon the user interface to know when to stop manually operating the pump to expel the air or gas from the reservoir (e.g., an indicator light turns green indicating gas is expelled and device is ready for drug administration).

In some embodiments, expelling air from the reservoir comprises engaging the pump mechanism.

In some embodiments, expelling the air from the reservoir comprises driving the air through an injection needle.

In some embodiments, expelling the air from the reservoir comprises driving through a membrane, wherein the membrane is permeable to air and impermeable to fluids. In some embodiments, the membrane comprises a sensor configured to detect when fluid contacts the membrane and stop the pump mechanism. In some embodiments, the membrane is a hydrophobic membrane. In some embodiments, the membrane comprises microporous hydrophobic membranes. In some embodiments, the membrane comprises polytetrafluorethylene, polypropylene, polyvinylidene difluoride, an acrylic polymer, or any other suitable membrane. In some embodiments, the membrane is molded to form a plug. In some embodiments, the membrane forms a plug in the fluid path of a needle configured to deliver the drug formulation to a subject.

In some embodiments, the drug delivery device further comprises an accelerometer and/or positional sensor configured to detect the orientation information of the outlet. In some embodiments, the accelerometer and/or positional sensor comprises a triaxial accelerometer, a triaxial gyroscope, a triaxial geomagnetic sensor, or any combination thereof.

In some embodiments, the accelerometer and/or positional sensor comprise a microchip integrated into an electronic system of the drug delivery device. Non-limiting examples of accelerometer and/or positional sensor include the Bosch Sensortec BNO055, the TDK MPU-9250, the TDK MPU-6050, and the MCube MC6470.

In some embodiments, the indicator comprises a light or graphical display. In some embodiments, the indicator comprises a light display. In some embodiments, the light display comprises a multiple segment light-bar configured to convey orientation information.

In some embodiments, the indicator comprises a graphical display. In some embodiments, the graphical display further displays instructions for the user.

The user interface may also comprise any of the elements described in the “Interfaces” section or configured as any of the embodiments described in the “Interfaces” section. In some embodiments, the user interface is attached to the device.

In some embodiments, the user interface is a wireless enabled device in wireless communication with the drug delivery device.

In some embodiments, the user interface further comprises additional information about the drug delivery device and/or the drug formulation.

In some embodiments, the user interface allows a subject to self-administer the dose of the drug formulation.

In some embodiments, the drug delivery device is a single component fully integrated device (e.g., single-part pen injector). In some embodiments, the drug delivery device is a multi-component device assembled by a user (e.g., two-part pen injector).

In some embodiments, the drug delivery device is configured for titrated delivery. In some embodiments, the drug delivery device is configured to deliver the drug formulation over a pre-determined period of time. In some embodiments, the drug delivery device is configured for intramuscular or subcutaneous administration of the drug formulation. In some embodiments, the drug delivery device is pre-filled and/or pre-loaded.

### Interfaces

The user interfaces provided herein can take a variety of forms and can be configured to display any information pertinent. The user interfaces provided herein can comprise physical displays directly on the device (e.g., a graphical display on the device or embedded lights, such as LEDs) that can be used to convey information to the user. Additionally, the user interface can comprise actionable components (e.g., buttons, knobs, switches, and the like) that are configured to perform force the device to perform one or more actions, such as to activate a pump mechanism, eject a cartridge or reservoir component from the device, or turn the device on or off.

The user interfaces herein can also comprise a graphical display on a device external to the drug delivery devices provided herein. In some embodiments, the user interface components provided herein do not comprise the user interface directly, but rather are in wireless communication with a user interface on an external device which is enabled for wireless communication with the user interface component. Non-limiting examples of such wireless communication enabling technologies include Low-Power-Bluetooth (LPB), WiFi, or other telemetry protocols. When the graphical display is on a device external to the drug delivery device, the graphical display may be run through any of a non-transitory computer readable storage medium containing software for the graphical display, a computer program, a web application, a mobile application, a standalone application, a software module, or a web-browser plug in.

In some embodiments, graphical displays of the user interface are configured to text, figures, or other graphics and combinations thereof. In some embodiments, the graphical display shows instructions to the user. In some embodiments, the graphical display shows text. In some embodiments, the graphical display is configured to show information about the device, the drug formulation, or a combination thereof. In some embodiments, the information includes manufacturing information, expiration date information, drug concentration information, the dosage regimen, or any other relevant information.

In some embodiments, the user interface comprises an indicator of reservoir fill status. The indicator of reservoir fill status displays information to the user about the volume of the drug formulation remaining in the reservoir. Any type of indicator capable of imparting this information to the user can be employed. In some embodiments, the fill status is displayed on a graphical display. In some embodiments, the fill status is displayed on a light bar. In some embodiments, the number of lights on the light bar emitting light at a given time corresponds to the proportion of drug formulation remaining in the reservoir. For example, a light bar having five lights can have three lights turned on when the reservoir is at about 60% of drug formulation remaining. Such an indicator would be especially useful in a drug delivery device where the reservoir is not visible.

In some embodiments, the user interface comprises an indicator of availability of bolus addition of drug product in a treatment regimen. In some embodiments, a light bar with a plurality of lights indicates the number of available bolus additions available, where the number of bolus additions available matches the number of lights turned on. In some embodiments, a color of a light display indicates if a bolus delivery is available according to a pre-determined dose regimen. Alternatively, availability of bolus information could be displayed as a color of a bolus indicator light. For example, a bolus indicator light being green could indicate that the dosage regimen currently allows a bolus addition and the bolus indicator light being red could indicate that the dosage regimen does not currently allow a bolus addition.

In some embodiments, the user interface comprises an indicator configured to display orientation information. In some embodiments, the orientation information is of an outlet of the reservoir. Such a display is especially useful for use with a priming method provided herein. In some embodiments, the indicator of orientation status comprises a light bar configured to show orientation information, including level information of the device. In some embodiments, the orientation status is a direction status, such as upward or downward. In some embodiments, the light bar is configured to show when the outlet of the reservoir is in an upward orientation. In some embodiments, the light bar has different lights turned on when the outlet of the reservoir is within a certain range parameter of the desired orientation. As a non-limiting example, such a display could have one light configuration when the device is within 5° of the desired orientation, another light configuration when the device is from 5°-30° of the desired orientation, or and a third light configuration when the device is greater than 30° from the desired orientation. In some embodiments, such information could be shown in a graphical interface in any one of a variety of manners, for example displaying a digital image of a spirit level.

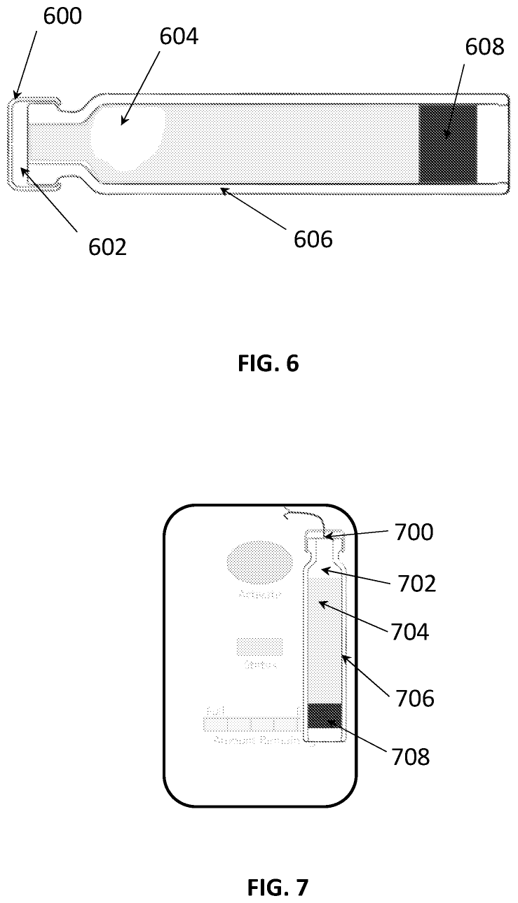

FIG. 6 shows a non-limiting embodiment of a delivery device with the crimp cap 600 and septum 602 covering the cartridge reservoir 606 and the plunger 608. As is shown in FIG. 6, a device containing a reservoir pre-filled with the drug formulation can contain air 604 as a result of the manufacturing process used to fill the reservoir. Pre-filled devices with reservoirs positioned on the interior of a device pose a special problem. This is because in order to expel trapped air, the device must be oriented such that the exit port of the reservoir is positioned up, thereby allowing the device to drive the air out of the reservoir without wasting the drug formulation disposed therein, as shown in FIG. 7. The plunger 708 can be used to release any trapped air 702 through the needle exit port 700. However, the reservoir 706 would need to be positioned properly to avoid releasing any of the medication/formulation 704. Because the reservoir is positioned on the interior of the device, a user seeking to prime the device cannot see when the device is properly positioned to drive air out of the device.

To address this and ensure the device is in the proper orientation prior to priming the system to expel trapped air from the reservoir, the user interface in this embodiment comprises a light bar functioning as a levelling indicator. The light bar is configured such that each light can be red, green, or yellow. During initiation of the device and before it is applied to the subject, the device is primed to remove any air that is trapped in the drug formulation reservoir during manufacturing. To ensure the device is properly oriented, the light bar is configured to act as a levelling indicator for this priming step. During this priming step, the light bar is configured to display orientation information about the reservoir within the device. A non-limiting embodiment of one such configuration is shown in FIG. 9. In the example shown in FIG. 9, the light bar is configured to display different combinations and colors of light in order to communicate orientation information to the subject. When the device is within a pre-determined threshold of the proper orientation (e.g., within a 5° angle of vertical orientation), the single middle light of the light bar shows a green signal. When the device is within a second pre-determined threshold of the proper orientation (e.g., from a 5° angle from vertical orientation to a 30° angle from vertical orientation), the two lights adjacent from the middle light of the light bar show a yellow signal. When the device is outside either of the pre-determined thresholds of the proper orientation (e.g., greater than a 30° angle from vertical orientation), the exterior lights of the light bar show a red signal. In this example, the orientation information is determined by a smart sensor (e.g., a Bosch Sensortec BNO055 smart sensor).

The accelerometer of this smart sensor is configured to monitor for excessive movements during transportation or manufacturing of the device which may have caused any trapped air to break up into smaller bubbles. If acceleration or force has exceeded a pre-determined threshold prior to administration by the patient, the pump status light displays a red color during the priming step until sufficient time in the proper orientation has passed for the bubbles to coalesce, such sufficient time being pre-determined based on the acceleration measured. Once both the pump status light and orientation lights are green, indicating the system is prepared for priming, priming of the system begins automatically to purge the trapped air.

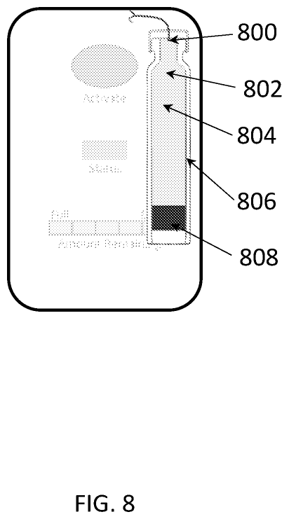

The automatic purging of the trapped air continues until all of the air is removed from the device, at which point the pump mechanism automatically turns off. In order to sense when the device has expelled all of the air from the reservoir, the pump mechanism is equipped with a force or pressure sensor which measures the amount of force being applied to drive the air out of the outlet of the reservoir. As driving air or the liquid drug formulation from the reservoir will require different amounts of pressure or force, measuring the pressure or force applied during this step provides an indication of what is being driven from the reservoir at a specific point in time. Thus, the pressure or force sensor is configured to stop the pump when the pressure or force corresponding to the amount of pressure or force required to drive liquid through the exit port is measured. FIG. 8 shows a drug reservoir that has been completely purged of air within a drug delivery device. In this example, the plunger 808 has been used to expel trapped air 802 through the needle exit port 800 after the reservoir 806 was positioned so that the air 802 was proximal to the needle exit port 800 to avoid releasing any of the medication/formulation 804.

### Drive System Lockout

On two or three component device designs that include a reusable component and one or more disposable components, there is a potential for unauthorized manipulation of the drive coupling interface as shown in FIG. 3 using a tool or the like and not through the intended reusable drive coupling interface. To reduce the likelihood of unintended manipulation of the drive coupling interface, pump designers will often select a drive coupling shape that is non-standard such as square, triangle, pentagon, hexagonal, heptagonal, star, or Torx. Certainly, a standard slotted or Philips head drive coupling interface would not be a preferred design. However, each of these drive coupling interfaces still provides the risk that a specialized tool could be used to manipulate the drive coupling interface. If unintended manipulation of the drive coupling of the disposable portion were to occur, there is a potential that some or all of the medication contained within the disposable component could be accessed or delivered without the intended controls provided from the reusable component. Even in the case of magnetic drive coupling interface as defined in FIG. 3B and FIG. 3E, a magnetic tool can be used to simulate the respective drive coupling interface within the reusable component.

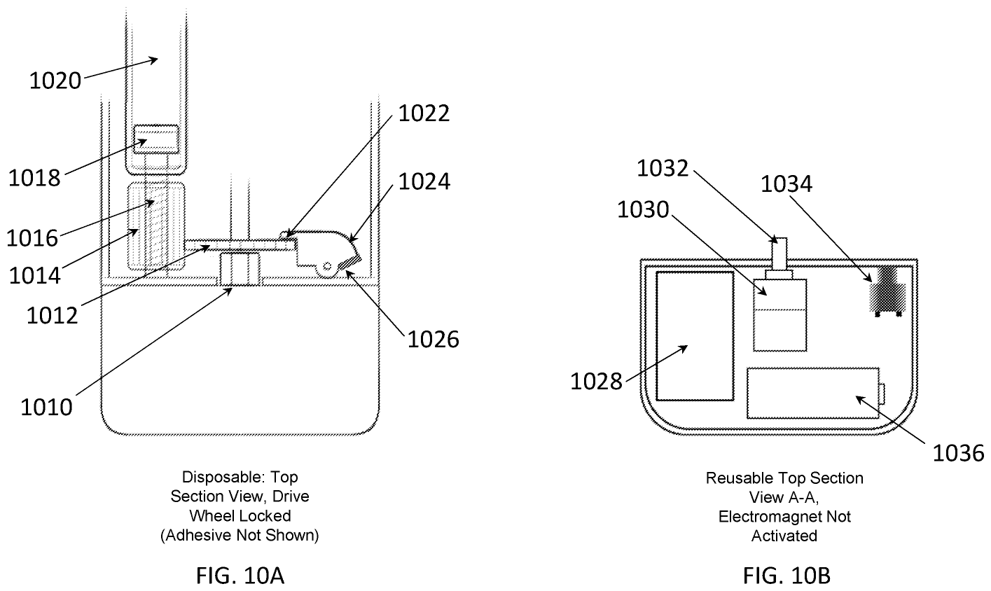

To address this potential and to ensure the drive system within the disposable component is not activated by means other than through the reusable component, some embodiments of devices disclosed herein comprise a drive system lockout mechanism that can keep the drive locked to prevent unauthorized administration or activation. As an illustrative and non-limiting example, a secondary drive wheel latch system is incorporated between the reusable component and the durable component, as shown in FIGS. 10A-10B. In one embodiment, an electromagnet, driven by the electronic board and control systems within the reusable component, is used to activate the drive wheel latch within the disposable component. FIGS. 10A-10B show the two-part pump in the locked state. In the absence of the magnetic force provided by the electromagnet upon the ferrous metal plate 1026, the drive wheel latch 1024 is in the normal latched state, not allowing the drive wheel 1012 to rotate by keeping the drive locked 1022, thereby inhibiting unintended rotation of the drive interface through the drive coupling nut as shown in FIG. 10A-10B. FIG. 10A also shows the disposable component with the primary medication container or reservoir 1020 with the cartridge plunger 1018, leadscrew 1016, and drive nut 1014, which is coupled to the drive wheel 1012 which is proximal to the drive coupling nut 1010. The reusable component is shown in FIG. 10B with the gear motor 1030, battery 1036, electronic module 1028, drive coupling shaft 1032 to the disposable coupling nut, and an electromagnet 1034 positioned in proximity to the ferrous metal plate 1026. In embodiments that utilize magnetic coupling between the reusable component and disposable component, the drive coupling interface is locked by the drive wheel latch until the electromagnet is activated to release the drive wheel.

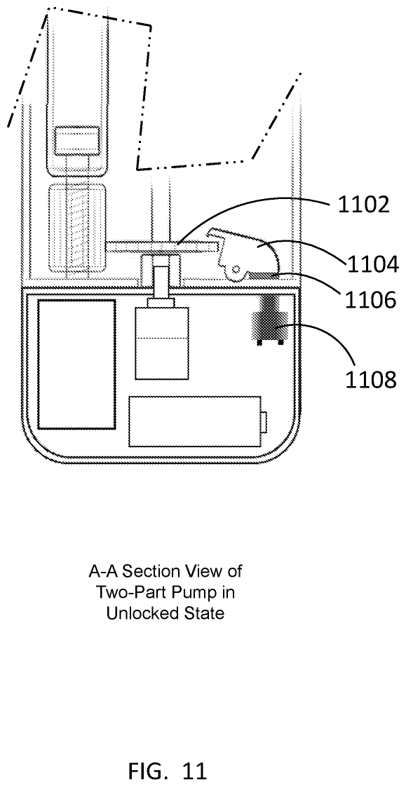

FIG. 11 shows a cross-section view of a non-limiting embodiment of a two-part pump device in the unlocked state. The drive wheel latch 1104 is in the unlocked state due to the magnetic pull upon the ferrous metal plate 1106 by the electromagnet 1108 within the reusable component. In this unlocked state, the drive wheel latch 1104 has released the drive wheel 1102, thereby allowing the rotation and activation of the drivetrain and plunger drive leadscrew. In other embodiments, the electromagnet within the reusable component is replaced with a permanent magnet that performs the function of pulling the ferrous metal plate, thereby activating the drive wheel latch and releasing the drive wheel when the reusable component is connected to the disposable component. This approach simplifies the design and lowers the cost of the device.

In further embodiments, other mechanisms are used to lock and unlock the drivetrain within the disposable component by active control within the reusable component. As an example, the disposable component could contain a drive latch that is activated by electronic control such as RFID, Near Field Communication (NFC), radio frequency, or the like. The disposable component could contain a small circuit and power system to activate a locking mechanism to inhibit the drivetrain within the disposable component. Alternatively, the power needed to drive the circuit or other type mechanism within the disposable component could be driven by an inductance coil within the reusable component and picked up by a receiving coil within the disposable component. Energy can be transferred from the reusable component to the disposable component by providing an alternating electromagnetic field. The benefit of an electronic and software controlled interface between the reusable component and this disposable component is the additional security and tamper resistance this interface provides to inhibit unintended activation of the drivetrain resulting in unprescribed medicinal fluid delivery.

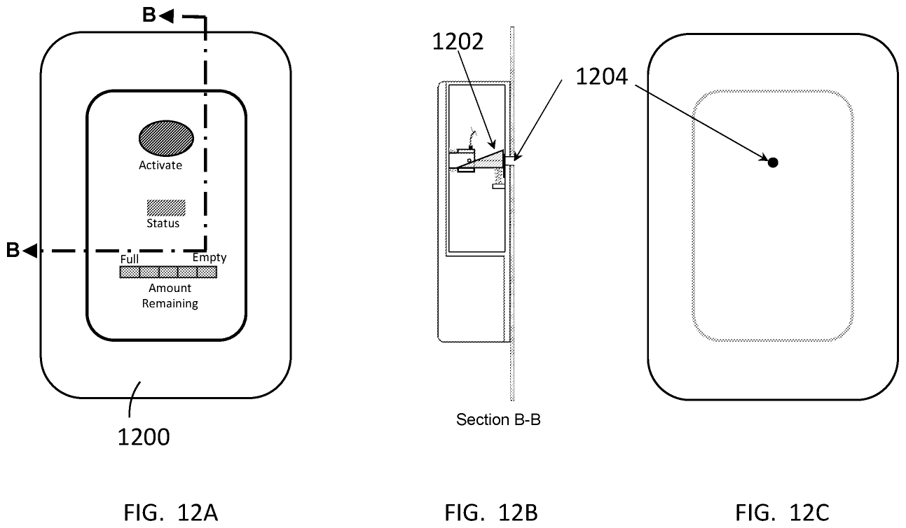

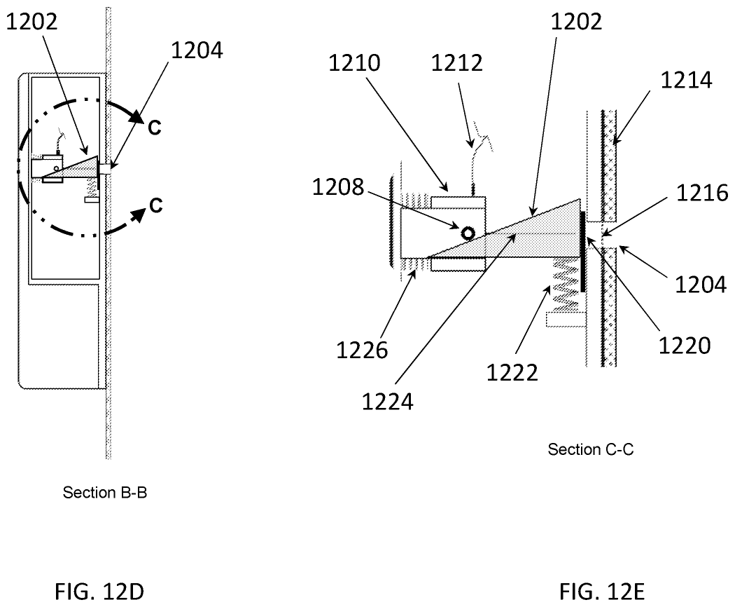

A benefit of a wearable delivery device is the portability and ability to deliver over longer periods of time, from minutes to days. However, for delivery of high value or controlled substances, it is important to inhibit access to the drug and ensure the drug is delivered only as prescribed. Therefore, several steps can be taken to inhibit access to the drug, such as having the drug prefilled primary container assembled and sealed within the device at the time of manufacture. This can be done with both a single integrated delivery pump or a pump system that has more than one component, such as a two part design that incorporates a reusable and disposable component. Having the drug prefilled and preloaded, and sealed within the disposable component limits access to the drug. Several additional steps could be taken to inhibit unintended delivery or access to the drug such as locking the drive coupling interface as shown in FIG. 10 and by utilizing software controls to deliver only as prescribed. However, for delivery to occur, the fluid path must be connected to the primary container providing for the flow of drug through the fluid path. Depending on the design of the pump, the patient needle end of the fluid path will, at some point, extend outside of the pump allowing for delivery into the patient. However, once the fluid path 1212 (FIG. 12D) is connected to the primary container allowing drug to flow into the fluid path, there is a risk that unintended access to the drug could be obtained by withdrawing drug from the patient needle end. FIG. 12A shows a top view of a non-limiting embodiment of the wearable device, while FIG. 12C shows a bottom view with the delivery port covered 1204 such that the patient needle is not extended. In the wearable device that has not yet extended the patient needle, withdrawal of the medication through the retracted patent needle could be accomplished by passing a tube or other means through the delivery port 1204 (water barrier membrane not shown) of the pump 1200, as shown in FIG. 12C, to withdraw or suck the medication from the primary container by vacuum. For example, this could be accomplished by using a tube connected to an empty syringe.

In order to inhibit access to the patient needle within the pump, a needle protection system 1202 is incorporated as shown in FIG. 12B and FIG. 12D with a close-up cross-sectional view shown in FIG. 12E. The needle protection system 1202 incorporates a needle protection door 1220 that blocks access to the retracted patient needle 1224 until the needle insertion mechanism 1210 is activated. A drug fluid path 1212 connects the drug reservoir containing the formulation to the needle 1224. Once the needle insertion mechanism 1210 is activated, the needle insertion spring 1226 drives the patient needle 1224 and needle insertion cam driver 1208 forward, resulting in the displacement of the needle protection door 1220 through the motion of the needle protection cam 1202. Also shown in FIG. 12E are the adhesive patch 1214, the water barrier membrane 1216, and the needle protection return spring 1222. This design, therefore, blocks access to the patient needle 1224 until the needle insertion mechanism 1210 is activated. The needle protection door 1220 automatically displaces during the needle insertion process allowing for the needle 1224 to extend from the pump through the port 1204 and insert into the patient's body.

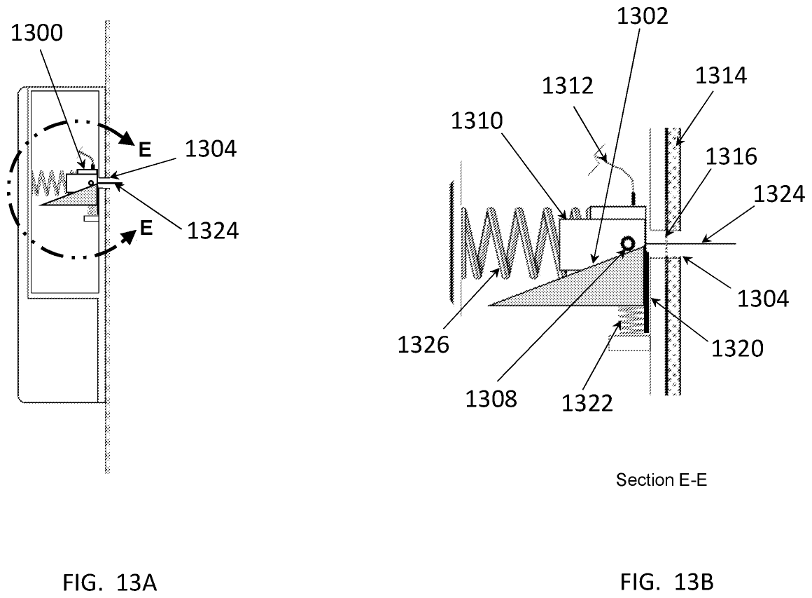

FIG. 13 shows the state in which the needle insertion mechanism has fully activated, thereby extending the patient needle to the full travel of displacement. FIG. 13A shows a side cross-sectional view of the needle protection system 1300 with the delivery port uncovered 1304 with the patient needle extended 1324. FIG. 13B shows a close-up E-E cross-sectional view bottom view with the delivery port uncovered 1304 and the patient needle extended 1324. The needle protection system 1300 incorporates a needle protection door 1320 that is shown in the open state after the needle insertion mechanism 1310 is activated to extend the patient needle 1324 through the delivery port 1304. A drug fluid path 1312 connects the drug reservoir containing the formulation to the needle 1324. The activated needle insertion mechanism 1310 causes the needle insertion spring 1326 drive the patient needle 1324 and needle insertion cam driver 1308 forward, which has displaced the needle protection door 1320 through the motion of the needle protection cam 1302. Also shown in FIG. 13B are the adhesive patch 1314, the water barrier membrane 1316, and the needle protection return spring 1322. Therefore, activation of the needle insertion mechanism 1310 results in the automatic displacement of the needle protection door 1320 and allows the needle 1324 to extend from the pump through the delivery port 1304 and insert into the patient's body.

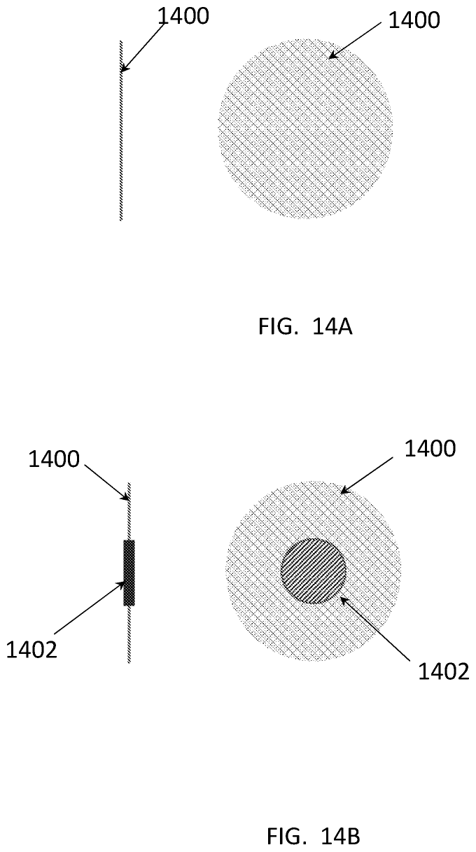

Wearable pump designs, either single or multiple components that remain watertight during and after exposer to water, as may occur during a shower or bath, exposed to rain, or during surface swimming, provides the benefit that the internal components remain dry. Certainly, watertightness is an important capability for pumps that utilized electronic components as part of the system's functionality. If these components were to become wet, there is a risk that they will malfunction and impact the normal use of the pump. Even mechanical only systems can be negatively impacted by water intrusion into the pump. Therefore, it is important to consider all points in which water can enter the interior of the pump. For single component devices, there are commonly used design and manufacturing processes to ensure the interior of the device remains dry during use. For example, the exterior housing that contains all the device components could be assembled and sealed using sonic welding, laser welding, or adhesives that limit water intrusion. The user interface and display could be sealed with the use of overlays (silicone, rubber, thin plastic, flexible membrane), second or two shot molding, welded light pipes, or thin wall sections within the housing. These technologies are commonly used to provide a watertight enclosure. However, for some enclosure designs, there is a benefit to have air pass through the enclosure to equalize pressure between the inside of the device and the surrounding environment. The ability to equalize pressure is a common need for drug delivery devices such as a wearable pump since a pressure differential could impact the drug delivery accuracy and could potentially result in unintended drug delivery into the patient. Therefore, to ensure that air can enter into the pump but water cannot, a feature is added to the enclosure wall that allows air to pass and not water. This feature can take the form of a water barrier membrane made from hydrophobic materials. Water barrier membranes with hydrophobic properties can comprise materials made from polytetrafluoroethylene, polypropylene, polyvinylidene difluoride, an acrylic polymer, or other suitable hydrophobic materials. The water barrier membrane can be a single part membrane 1400 as shown in FIG. 14A with a side view and a top view. The water barrier membrane as shown in FIG. 14A could be located at any location within the drug delivery device that allows air to pass and not water such that the relative pressure equalizes between internal chambers within the delivery device and the external environment. In some commercial designs, this type of water barrier membrane can be located in the exterior pump housing, adjacent to the adhesive pad that attaches a wearable pump to the patients body, or in the disposable infusion set housing. However, wearable drug delivery devices must also have the ability to pass the patient needle from the interior of the pump housing to the exterior in order to enable drug delivery into the patient. This delivery port can be seen in FIG. 12 and FIG. 13A-13B. However, to ensure that water does not enter into the pump enclosure, a water barrier membrane can be added at the delivery port that will allow air to pass and equalize the relative pressure between the inside of the pump and the exterior environment while not allowing water to ingress into the pump. In this embodiment, the water barrier membrane serves to maintain watertightness prior to and after needle activation. During the needle activation process, the patient needle will penetrate the water barrier membrane and extend from the pump, enabling drug delivery into the patient. In this embodiment, the water barrier member will have the property to seal around the patient needle to maintain the water barrier function to inhibit water entry into the pump. FIGS. 12E, 13B, 14A show an example of a water barrier membrane at the delivery port within the wearable pump enclosure. The geometry of the material that performs the intended water barrier function does not need to be a thin film. For example, the water barrier can be made from materials with a range of thicknesses up to 5 millimeters or more, or be cylindrical in shape, or have multiple different shapes. The general property is that the water barrier component is selectively permeable such that it allows air to pass and not water.

Alternatively, in some embodiments, the water barrier membrane is constructed from a composite of materials that perform multiple functions, as shown in FIG. 14B. In this embodiment, the composite is composed of a hydrophobic water barrier membrane 1400 to perform the function of allowing air to pass but not water, and a needle sealing barrier 1402 that is designed to ensure a watertight seal between the water barrier membrane and the patient needle. The needle sealing barrier 1402 could be made from silicone, low durometer polyethylene, butyl rubber, or high density foam. The function of the needle sealing barrier 1402 is to allow the patient needle to penetrate it during the needle insertion process while maintaining a watertight barrier properties at their interface.

In yet another embodiment, the function of the water tight barrier at the delivery port and the watertight sealing between the patient needle and the enclosure is separated into two or more components. A vent hole used in conjunction with hydrophobic material can be provided on any surface of the enclosure to serve the function of allowing air to pass into the enclosure and not water. In addition, the delivery port can utilize a needle sealing barrier that does not allow air or water to pass, such as with the use of silicone, low durometer polyethylene, butyl rubber, high density foam, or the composite of more than one material.

In other embodiments, the needle protection system and water barrier membrane as described within a single component pump is implemented in any component of a multi-component pump such as a two component delivery system.

It is common for delivery devices such as pen injectors, autoinjectors, or wearable pumps to incorporate a primary medicinal container that is made from glass. In some cases, this primary container is prefilled and preloaded at the time of manufacture, and in some cases the primary container is prefilled or user filled and loaded into the delivery device by the patient, healthcare provider, pharmacist, or other person. However, glass is a brittle material and can fracture if impacted with sufficient energy. Additionally, a primary container assembly within a delivery system typically incorporates more than one component such as a glass cartridge, rubber plunger, rubber septum, and aluminum crimp cap to hold the rubber septum in place as shown in FIG. 6. If the glass cartridge within a delivery device, such as but not limited to a wearable pump, the medicinal contents will leak within the device and may also leak outside of the device. In some cases, the container closure integrity is lost at the interface between adjoining components, such as between the rubber septum and the glass cartridge container. If this occurs, the medicine can also leak within the delivery system. The leaked medicinal fluid could damage components within the device, including electronic components. For example, if electronic components were damaged by this fluid, the electronics could malfunction and be unable to inform the user of the error condition thereby not actively informing the patient that delivery has stopped or is impacted. In other embodiments, the glass primary container could be made from other materials such as Crystal Zenith, Cyclic Olefin Copolymer, Cyclic Olefin Polymer, or other material that meet the compatibility requirements of the drug to be contained within. If the medicine contained within the delivery device is of high value or is a controlled substance, someone may attempt to gain access to the drug by damaging the primary container such that it would leak out of the device. Damage or access to the drug could be accomplished by breaking the primary container or one of its components, or by drilling into the primary container with the intent to access the drug.

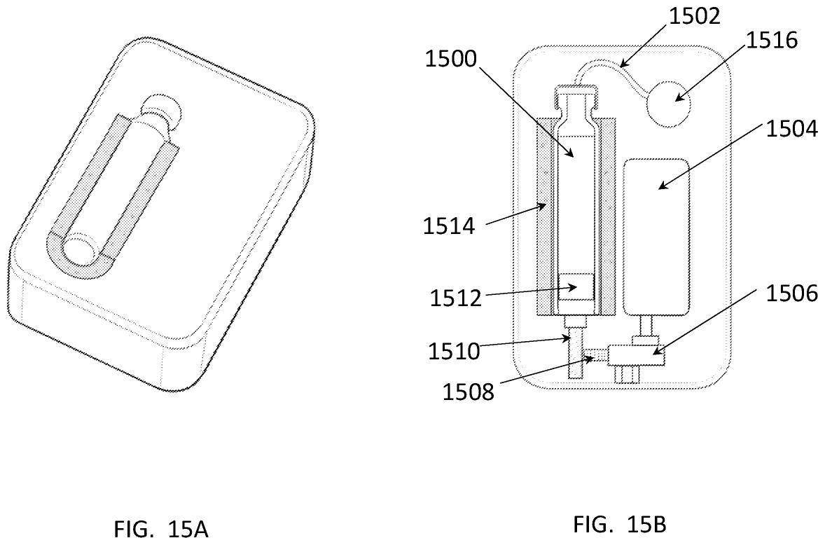

To inhibit access to the drug within the delivery device, a liquid absorbing material could be placed around the primary container (e.g., cartridge) 1500 that will soak up or otherwise change the form or efficacy of the drug such that it becomes unusable or inaccessible. FIG. 15 shows an example of liquid absorbing material 1514 in the proximity of the primary container 1500 that will absorb or alter the drug leaked from the primary container. The liquid absorbing material 1514 can comprise an absorbing sponge made from one or more absorbent materials such as polyester, polyurethane, or vegetal cellulose. These materials can soak up leaked fluids from the primary container 1500 to inhibit access. In some embodiments, the liquid absorbing material 1514 is a material that converts the liquid into a solid or gelatin material such as slush powder, which can be made from a material such as sodium polyacrylate. Slush powder is a super absorbent chemical that can absorb up to 1,000 times its weight of the fluid. Another non-limiting example of a fluid absorbing material is silicon dioxide that could perform a similar function. These materials can also absorb excess humidity that might be present within the delivery device. In this example, if slush powder is placed around the cartridge container, leaked fluid will be converted into a gel, thereby inhibiting access to the drug. Also shown in FIG. 15B is the electronic module 1504 that controls a gearmotor 1506 to rotate the drive wheel 1508 to turn the leadscrew 1510 that acts upon the cartridge plunger 1512 to push the formulation from the cartridge container 1500 through the fluid flow path 1502 to the patient needle insertion mechanism 1516.

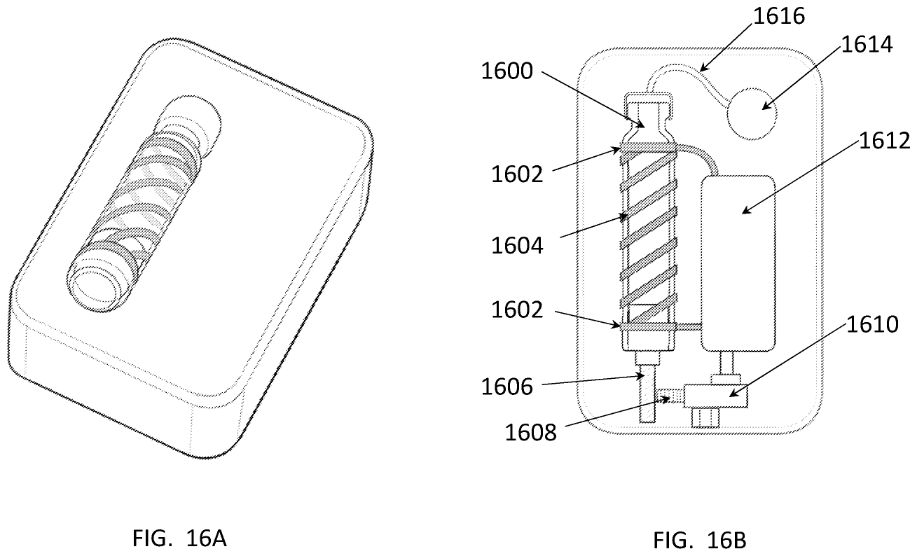

The design intent of drug delivery systems is for the device to deliver the contained medicine to the patient as programmed and within the design specifications and labeling of the device. However, drug delivery systems have components that can be damaged or broken due to physical impact such as a result of the device being dropped, bumped against an object such as a door frame, chair, or table. In some cases, the drug delivery device would become inoperable due to an impact. For example, the drug filled reservoir or fluid path could be damaged by impact such that the medicinal content can be partially or fully leaked from the reservoir or fluid path. Additionally, drug delivery devices can be damaged by intentional impact from tools such as a hammer, pliers, wrench, screwdrivers or the like. It is important for drug delivery systems to incorporate design features to inform the user of potential damaged to the drug delivery device that might inhibit its ability to deliver and designed. For delivery devices that incorporate a glass or breakable primary container, special care must be taken to ensure this component is not damaged or broken resulting in drug leakage within the delivery system or from the delivery system. In some designs, a compliant material is incorporated between the drug filled reservoir and the mounting features within the drug delivery device with the intent to absorb and lessen impact energy that could result in reservoir damage and drug leakage. However, impacts of sufficient energy or by intended misuse such as drilling probing, or poking can still result in reservoir damage and therefore resulting drug leakage. In some embodiments, for container materials made of a breakable material such as glass, a conductive trace can be implemented on or around the container that is connected to the electronic module within the device. If the container were to be damaged, the conductive trace would be affected such that its change in properties could be measured by the electronic module. In one example, a conductive trace is placed around the primary container and connected to the electronics as shown in FIG. 16, such that if the container were to be damaged and the trace is partially or fully broken, the electronic module would sense the change in the trace impedance and provide notification to the user and stop delivery. FIG. 16A shows an isometric view of the device, while FIG. 16B shows a cross-section view with the conductive trace 1604 wrapped around the cartridge 1600 with conductive trace contacts 1602 that connect to the electronic module 1612. Thus, the electronic module 1612, upon detecting the damage to the container via change in trace impedance, can stop controlling the gearmotor 1610 to rotate the drive wheel 1608 turning the leadscrew 1606 to push/pump the formulation from the cartridge 1600 through the fluid flow path 1616 into the patient needle insertion mechanism 1614.

Delivery devices such as ambulatory, portable, or wearable pumps are designed to deliver the medication as instructed and programmed. Route of administration includes Intravenous, intramuscular, and subcutaneous. In all these delivery systems, there is tubing, needle, or port that transfers medicine from the delivery device to the patient. When therapy has been initiated, the fluid path from the drug primary container to the patient has been established. In some systems, once the fluid path is established, it remains open through the use and end of use of the delivery system. End of use could occur as a result of the drug being delivered fully, the intended therapy has ended, and some drug remains within the delivery device due to a system error state or due to premature termination of therapy. Consider a prefilled and preloaded wearable delivery device such as that shown in FIG. 2 or FIG. 3A-3B. For delivery to occur within the patient, the device must first establish an open fluid path between the medicine within the primary container, such as a cartridge container and the patient. For subcutaneous delivery, the fluid path terminates distally with a patient needle or cannula inserted into the patient's subcutaneous tissue. In order to maintain sterility of the primary container during storage and prior to use, the primary container is designed with materials and manufacturing processes to ensure sterility. When the time comes to enable delivery, a means is provided to access the medicine within the primary container. On staked needle syringe based primary container systems, an elastomeric material demonstrated to keep the medicine sterile is removed from the needle, thereby opening the fluid path. However, on cartridge based primary container systems, the sterile closure is maintained by an elastomeric plunger and septum, as shown in FIG. 6. To enable delivery with a cartridge based system, the cartridge needle on the proximal end of the fluid path must penetrate the cartridge septum, thereby allowing fluid to flow through the fluid path into the patent.

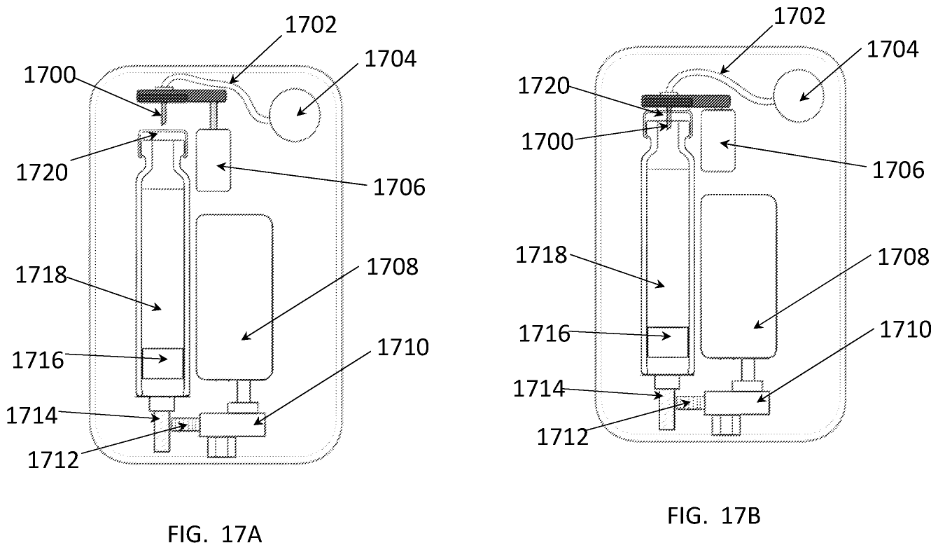

FIG. 17A-17B shows a wearable pump that incorporates a cartridge primary container 1718, a drive system (e.g., gearmotor 1710, drive wheel 1712, and leadscrew 1714) to displace the cartridge plunger 1716, a patient needle insertion mechanism 1704, a fluid path 1702 that includes both a patient needle (not shown) and cartridge needle 1700, and a solenoid 1706 used to cause the cartridge needle 1700 to penetrate the cartridge septum 1720. In FIG. 17A, the cartridge needle 1700 has not penetrated the cartridge septum 1720, and the cartridge assembly 1718 remains sterile. When the solenoid 1706 is retracted, it translates the cartridge needle 1700 causing it to penetrate the cartridge septum 1720 and thereby allowing fluid to flow into the fluid path 1702 as shown in FIG. 17B. In order to deliver the medication into the patient, a delivery port within the delivery device housing is needed as shown in FIGS. 12B-12C. This delivery port allows the patient needle to pass and enter the patient's skin enabling subcutaneous or intramuscular delivery.

Since the fluid path from the primary container is open, as shown in FIG. 17B, to the patient needle, unintended access to the drug could occur by accessing the patient needle, through the delivery port hole, with a tube and method to suck and withdraw the drug from the primary container. In the case where the patient needle has been extended from the pump either before, during, or after therapy, a similar means could be used to withdraw the medication from the patient needle. If the patient needle is exposed, an empty cartridge with a plunger rod could be used to withdraw the medication from the primary container.

In some embodiments, in order to inhibit access to the drug after the fluid path has been established, the cartridge needle could be withdrawn from the cartridge by command from the electronic module to extend the cartridge needle by extension of the solenoid as shown in FIG. 17A. The solenoid can be actuated to extend as a result of any of several conditions including, for example, one or more of end of deliverable medicine within the primary container, end of intended therapy, error state, premature removal of the delivery device from the body, or by other commands. When the solenoid is in the extended state, as shown in FIG. 17A, medicine is blocked from flowing into the fluid path from the primary container and therefore inhibiting unintentional access to the drug within the primary container.

The activation of the needle 1700 to penetrate the cartridge septum 1720 or to withdraw from the cartridge septum 1720 can be operated by a solenoid as described herein or by multiple other drive mechanisms that result in the translation of the needle 1700 into and from the cartridge septum 1720. Other drive mechanisms include a motor driven cam or leadscrew and drive nut assembly that due to motor rotation, the cam interface to the needle 1700 or needle support components result in the translation into and out of the cartridge septum 1720. In other embodiments, a linear motor can be coupled to the needle or needle support components resulting result in the translation into and out of the cartridge septum 1720. In other embodiments, a Nitinol wire can be coupled to the needle or needle support components and powered by the electronic module 1708 resulting result in the translation into or out of the cartridge septum 1720.

In other embodiments, the action of moving the needle 1700 into the cartridge septum 1720 can be driven by the mechanical components used to deliver the medication from the reservoir. In this design, the rotary and/or translating mechanisms that are operated to drive the reservoir plunger can be also coupled to the needle 1700 or needle support components such that the drive mechanisms first translate the needle 1700 into the reservoir septum 1720 before or during the mechanical movement that results in displacement of the cartridge plunger 1716. Furthermore, the rotary and/or translating mechanism can also activate the patient needle insertion mechanism 1704. This design provides the benefit that a single device activation by the user results in opening the fluid path by inserting the needle 1700 into the cartridge septum 1720, displaces the cartridge plunger 1716 through the action of leadscrew 1714, priming the fluid path 1702, and inserting the patient needle 1704 automatically.

In some embodiments, the needle activation using the rotary and/or translating mechanisms can be used to automatically disconnect the fluid path needle 1700 from the cartridge septum 1720 after the intended delivery is complete or upon cessation of therapy. In some embodiments, in addition to disconnecting the fluid path needle 1700 from the cartridge septum 1720 after the intended delivery is complete or upon cessation of therapy, the rotary and/or translating mechanisms can also automatically retract the patient needle 1704 providing needle stick protection when the device is removed from the patients body.

Once properly primed and all the air removed from the system, the subject applies the device to their body and beings the treatment regimen.

### Intramuscular or Subcutaneous Injection

Described herein are systems, devices, and methods for delivery of drug formulations such as by intramuscular or subcutaneous injection that is not limited to the hospital or clinic setting. Intramuscular or subcutaneous injection avoids certain drawbacks found in oral, sublingual, nasal, and rectal modes of administration. Intramuscular or subcutaneous injection allows higher drug absorption by avoiding first pass metabolism. In the case of ketamine, intramuscular or subcutaneous infusion allows a higher proportion of the total delivered drug to remain in the active, effective form of racemic and/or s-ketamine (e.g., in an untransformed state) rather than biotransformation through first pass metabolism into less effective metabolites including but not limited to: S-norketamine, R-norketamine, S-dehydronorketamine, R-dehydronorketamine, 2S,6R-hydroxyketamine, 2R,6S-hydroxyketamine, 2S,6S-hydroxyketamine, and 2R,2S-hydroxyketamine. Accordingly, total body exposure to ketamine and to ketamine metabolites in the course of treatment is reduced compared to the current art by allowing lower total dosing per treatment. This decreases the burden placed upon the body in detoxification, thus reducing associated risks such as bladder dysfunction. Moreover, removal of first pass metabolism improves interpatient dosing range reliability in treatment by reducing the effects of interpatient variation in CYP3A and/or CYP2B6 and/or CYP 2C9 enzymes known to cause large variations in plasma concentration in administration with first pass metabolism. This can also reduce some of the interpatient variability in plasma concentration levels due to concurrent use of CYP3A and/or CYP2B6 and/or CYP 2C9 inhibitors, substrates or inducer.

In addition to ketamine, any number of drugs may be employed in the drug formulations of the devices provided herein. Non-limiting examples of other drugs which may be used in the devices provided herein include Schedule 1 drugs, Schedule 2 drugs, Schedule 3 drugs, Schedule 4 drugs, opioids, drugs with a high potential for abuse, drugs with a moderate potential for abuse, drugs with a high potential for addiction, drugs with a moderate potential for addiction drugs with a high resale value, any controlled substance, or any drug for which titratable, sustained delivery is required.

### Programmed Dosage Regimen

Described herein are programmed dosage regimens for use with the systems, devices, and methods of the instant disclosure. In some embodiments, a dosage regimen comprises a series of doses. In some embodiments, a dosage regimen comprises a plurality of dosing options selectable by the subject and/or user. For example, a dosage regimen comprises three selectable dose options: a single continuous infusion dose at 1 mg/hour, a single continuous infusion dose at 2 mg/hour, and a low bolus injection of 1 mg that repeats every hour. In some embodiments, a dosage regimen comprises at least 1, 2, 3, 4, 5, 6, 7, 8, 9, 10, 11, 12, 13, 14, 15, 16, 17, 18, 19, or 20 or more dosing options and/or no more than 1, 2, 3, 4, 5, 6, 7, 8, 9, 10, 11, 12, 13, 14, 15, 16, 17, 18, 19, or 20 or more dosing options. In some embodiments, a dosage regimen comprises one or more dosage limits. In some embodiments, a dosage regimen comprises dosage duration (e.g., time period to infuse a single dose). In some embodiments, a dosage regimen comprises treatment duration (e.g., time period of entire dosage or treatment regimen). In some embodiments, the dosage regimen is configured for administration of a drug formulation comprising ketamine (e.g., ketamine HCl). In some embodiments, the dosage regimen is configured for administration of a drug formulation comprising any potentially abusable drug. Such potentially abusable drugs include any Schedule 1, Schedule 2, Schedule 3, or Schedule 4 drug suitable to be administered subcutaneously or intramuscularly. As used herein, Schedule 1, Schedule 2, Schedule 3, and Schedule 4 drugs refer to those so listed by the Drug Enforcement Agency of the United States Government. In some embodiments, a programmed dosage regimen comprises a continuous infusion dose. In some embodiments, a continuous infusion dose is optionally paused and continued according to user input. In some embodiments, a continuous infusion dose comprises an infusion rate of at least 0.01, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or at least 200 mg/hour and/or no more than 0, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or no more than 200 mg/hour of an active ingredient such as ketamine.

In some embodiments, a continuous infusion dose comprises an infusion rate of at least 0.0001, 0.001, 0.002, 0.003, 0.004, 0.005, 0.006, 0.007, 0.008, 0.009, 0.01, 0.015, 0.20, 0.025, 0.03, 0.035, 0.040, 0.045, 0.050, 0.055, 0.060, 0.065, 0.070, 0.075, 0.080, 0.085, 0.090, 0.095, 0.1, 0.11, 0.12, 0.13, 0.14, 0.15, 0.16, 0.17, 0.18, 0.19, or at least 0.2 milligrams/kg/hour or no more than 0.0001, 0.001, 0.002, 0.003, 0.004, 0.005, 0.006, 0.007, 0.008, 0.009, 0.01, 0.015, 0.20, 0.025, 0.03, 0.035, 0.040, 0.045, 0.050, 0.055, 0.060, 0.065, 0.070, 0.075, 0.080, 0.085, 0.090, 0.095, 0.1, 0.11, 0.12, 0.13, 0.14, 0.15, 0.16, 0.17, 0.18, 0.19, or at least 0.2 milligrams/kg/hour of an active ingredient such as ketamine. In some embodiments, a continuous infusion dose comprises an infusion rate range that is at least 0.0001, 0.001, 0.002, 0.003, 0.004, 0.005, 0.006, 0.007, 0.008, 0.009, 0.01, 0.015, 0.20, 0.025, 0.03, 0.035, 0.040, 0.045, 0.050, 0.055, 0.060, 0.065, 0.070, 0.075, 0.080, 0.085, 0.090, 0.095, 0.1, 0.11, 0.12, 0.13, 0.14, 0.15, 0.16, 0.17, 0.18, 0.19, or at least 0.2 milligrams/kg/hour and no more than 0.0001, 0.001, 0.002, 0.003, 0.004, 0.005, 0.006, 0.007, 0.008, 0.009, 0.01, 0.015, 0.20, 0.025, 0.03, 0.035, 0.040, 0.045, 0.050, 0.055, 0.060, 0.065, 0.070, 0.075, 0.080, 0.085, 0.090, 0.095, 0.1, 0.11, 0.12, 0.13, 0.14, 0.15, 0.16, 0.17, 0.18, 0.19, or at least 0.2 milligrams/kg/hour of an active ingredient such as ketamine.

In some embodiments, a programmed dosage regimen has an infusion duration (e.g., time to infuse a single dose). In some embodiments, a programmed dosage regimen has an infusion duration of at least 1, 2, 3, 4, 5, 6, 7, 8, 9, 10, 15, 20, 25, 30, 35, 40, 45, 50, 55, or 60 or more minutes, or at least 1, 1.5, 2.0, 2.5, 3.0, 3.5, 4.0, 4.5, 5.0, 6.0, 7.0, 8.0, 9.0, 11.0, 12.0, 13.0, 14.0, 15.0, 16.0, 17.0, 18.0, 19.0, 20.0, 21.0, 22.0, 23.0, or at least 24.0 hours or more, or at least 1, 2, 3, 4, 5, 6, 7, 8, 9, or 10 days or more. In some embodiments, a programmed dosage regimen has an infusion duration of no more than 1, 2, 3, 4, 5, 6, 7, 8, 9, 10, 15, 20, 25, 30, 35, 40, 45, 50, 55, or 60 minutes or more, or no more than 1, 1.5, 2.0, 2.5, 3.0, 3.5, 4.0, 4.5, 5.0, 6.0, 7.0, 8.0, 9.0, 11.0, 12.0, 13.0, 14.0, 15.0, 16.0, 17.0, 18.0, 19.0, 20.0, 21.0, 22.0, 23.0, or 24.0 hours or more, or no more than 1, 2, 3, 4, 5, 6, 7, 8, 9, or 10 days or more. In some embodiments, a programmed dosage regimen has an infusion duration that lasts until the drug formulation is depleted or almost depleted (e.g., over 80%, 85%, 90%, 95%, or 99% of the drug formulation in the drug reservoir or cartridge is depleted).

In some embodiments, a programmed dosage regimen comprises one or more doses. In some embodiments, a programmed dosage regimen comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 doses or more. In some embodiments, a programmed dosage regimen comprises no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 doses or more. In some embodiments, a programmed dosage regimen comprises an infusion rate range that is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 doses or more and no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 doses or more.

In some embodiments, a programmed dosage regimen comprises one or more doses per time period. In some embodiments, a programmed dosage regimen comprises at least 1, 2, 3, 4, 5, 6, 7, 8, 9, 10, 11, 12, 13, 14, 15, 16, 17, 18, 19, 20, 21, 22, 23, or 24 doses or more per 1, 2, 3, 4, 5, or 6 days, or per 1, 2, 3, 4, 5, 6, 7, or 8 weeks.

In some embodiments, a programmed dosage regimen has a treatment duration. For example, a treatment duration can be a month long treatment. In some embodiments, the treatment duration is indefinite (e.g., no set duration). In some embodiments, a programmed dosage regimen has a duration of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or more. In some embodiments, a programmed dosage regimen has a duration of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks or more. In some embodiments, a programmed dosage regimen has a duration of no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or more. In some embodiments, a programmed dosage regimen has a duration of no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks or more. In some embodiments, a programmed dosage regimen has a duration of between 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days and 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, a programmed dosage regimen has a duration of between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks and 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In some embodiments, a continuous infusion dose comprises an infusion rate range that is at least 0.01, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or at least 200 mg/hour and no more than 0.01, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 125, 130, 140, 150, 160, 170, 180, 190, or no more than 200 mg/hour of an active ingredient such as ketamine. In some embodiments, a continuous infusion dose is delivered at more than one infusion rate during the duration of the dose. In some embodiments, a continuous infusion dose is delivered at a variable infusion rate. In some embodiments, a continuous infusion dose is delivered at an infusion rate that is optionally variable by the subject (e.g., subject can adjust the infusion rate while the dose is being administered). In some embodiments, a continuous infusion dose is interruptible by the subject such as pausing or turning off the dosage regimen and/or device. For example, in some embodiments, a continuous infusion dose comprises a duration when the infusion rate is 0.0 mg/hour.

In some embodiments, a programmed dosage regimen comprises one or more dosage limits. For example, a programmed dosage regimen may be locked to allow a user some flexibility to adjust a dosage or infusion rate within preset thresholds set by the authorized user or doctor/healthcare provider. Accordingly, a doctor may set a ketamine infusion threshold between 0.1 mg/kg and 1 mg/kg within which a user can adjust his infusion rate, but is unable to reconfigure the dosage regimen itself (e.g., adjust the thresholds). In some embodiments, a programmed dosage regimen comprises an upper limit setting a maximum quantity of a drug formulation to be delivered. In some embodiments, a programmed dosage regimen comprises a lower limit setting a minimum quantity of a drug formulation to be delivered. In some embodiments, a dosage limit is configured by a doctor or healthcare provider. In some embodiments, a dosage limit is configured by an authorized user or a user who provides authentication information for unlocking a drug delivery device. In some embodiments, a programmed dosage regimen comprises a single dose limit (e.g., limit amount of drug delivered in a single dose). In some embodiments, a single dose limit is about 0.01, 0.1, 0.2, 0.25, 0.3, 0.4, 0.5, 0.6, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 125, 150, 200, 250, 300, 350, 400, 450, or at least 500 mg per dose. In some embodiments, a single dose limit is at least 0.01, 0.1, 0.2, 0.25, 0.3, 0.4, 0.5, 0.6, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 125, 150, 200, 250, 300, 350, 400, 450, or at least 500 mg per dose and/or is no more than 0.01, 0.1, 0.2, 0.25, 0.3, 0.4, 0.5, 0.6, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 125, 150, 200, 250, 300, 350, 400, 450, or at least 500 mg per dose.

In some embodiments, a single dose limit is about 0.001, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.60, 0.70, 0.80, 0.90, 1.0, 1.1, 1.2, 1.25, 1.3, 1.4, 1.50, 1.6, 1.7, 1.75, 1.8, 1.9, 2.00, 2.50, 3.00, 3.50, 4.00, 4.50, or at least 5.00 milligrams/kg/dose. In some embodiments, a single dose limit is about 1 milligrams/kg/dose. In some embodiments, a single dose limit is about 5 milligrams/kg/dose. In some embodiments, a single dose limit is at least 0, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.60, 0.70, 0.80, 0.90, 1.0, 1.1, 1.2, 1.25, 1.3, 1.4, 1.50, 1.6, 1.7, 1.75, 1.8, 1.9, 2.00, 2.50, 3.00, 3.50, 4.00, 4.50, or at least 5.00 milligrams/kg/dose and/or is no more than 0, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.60, 0.70, 0.80, 0.90, 1.0, 1.25, 1.50, 2.00, 2.50, 3.00, 3.50, 4.00, 4.50, or no more than 5.00 milligrams/kg/dose.

In some embodiments, a programmed dosage regimen comprises a daily dose limit (e.g., limit amount of drug delivered in a single day or 24 h). In some embodiments, a daily dose limit is about 0, 1, 2, 3, 4, 5, 10, 15, 20, 25, 30, 35, 40, 45, 50, 55, 60, 65, 70, 75, 80, 85, 90, 95, 100, 125, 150, 200, 250, 300, 350, 400, 450, or at least 500 mg per day. In some embodiments, a daily dose limit is at least 0, 1, 2, 3, 4, 5, 10, 15, 20, 25, 30, 35, 40, 45, 50, 55, 60, 65, 70, 75, 80, 85, 90, 95, 100, 125, 150, 200, 250, 300, 350, 400, 450, or at least 500 mg per day and/or is no more than 0, 1, 2, 3, 4, 5, 10, 15, 20, 25, 30, 35, 40, 45, 50, 55, 60, 65, 70, 75, 80, 85, 90, 95, 100, 125, 150, 200, 250, 300, 350, 400, 450, or no more than 500 mg per day. In some embodiments, a daily dose limit is about 125 mg per day. In some embodiments, a daily dose limit is about 200 mg per day.

In some embodiments, a daily dose limit is about 0, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.10, 1.20, 1.25, 1.30, 1.40, 1.50, 1.60, 1.70, 1.80, 1.90, 2.00, 2.50, 3.00, 3.50, 4.00, 4.50, or at least 5.00 milligrams/kg/day. In some embodiments, a daily dose limit is about 1 mg/kg/day. In some embodiments, a daily dose limit is about 5 mg/kg/day. In some embodiments, a daily dose limit is at least 0, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.10, 1.20, 1.25, 1.30, 1.40, 1.50, 1.60, 1.70, 1.80, 1.90, 2.00, 2.50, 3.00, 3.50, 4.00, 4.50, or at least 5.00 milligrams/kg/day and/or is no more than 0, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.10, 1.20, 1.25, 1.30, 1.40, 1.50, 1.60, 1.70, 1.80, 1.90, 2.00, 2.50, 3.00, 3.50, 4.00, 4.50, or at least 5.00 milligrams/kg/day.

In some embodiments, a programmed dosage regimen comprises a weekly dose limit (e.g., limit amount of drug delivered in a single week or 7 days). In some embodiments, a weekly dose limit is about 1, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or at least 1000 mg per week. In some embodiments, a weekly dose limit is at least 1, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or at least 1000 mg per week and/or is no more than 1, 5, 10, 15, 20, 25, 30, 35, 40, 45, 50, 60, 70, 80, 90, 100, 125, 150, 200, 250, 300, 350, 400, 450, 500, 550, 600, 650, 700, 750, 800, 850, 900, 950, or no more than 1000 per week.

In some embodiments, a weekly dose limit is about 0.25, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 200, 250, 300, 350, 400, 450, or at least 500 milligrams/kg/week. In some embodiments, a weekly dose limit is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, or at least 500 mg/kg/week and/or is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 200, 250, 300, 350, 400, 450, or no more than 500 mg/kg/week.

In some embodiments, a programmed dosage regimen provides a clinically effective steady state plasma concentration of the active ingredient such as ketamine within 1, 2, 3, 4, 5, 6, 7, 8, 9, 10, 11, 12, 13, 14, 15, 16, 17, 18, 19, 20, 21, 22, 23, 24 hours or more of treatment. In some embodiments, a programmed dosage regimen provides a clinically effective steady state plasma concentration of an active ingredient such as ketamine within 1, 2, 3, 4, 5, 6, 7, 8, 9, 10, 11, 12, 13, 14, 15, 16, 17, 18, 19, 20, 21, 22, 23, 24 hours or more of treatment outside of a hospital or clinic environment. In some embodiments, a programmed dosage regimen provides a steady state drug plasma concentration (e.g., ketamine) of at least 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL and/or no more than 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL.

In some embodiments, a programmed dosage regimen provides a clinically effective steady state plasma concentration of an active ingredient such as ketamine with a peak trough fluctuation that is lower than a comparable fluctuation from intravenous or intramuscular administration in a hospital or clinic setting. In some embodiments, a programmed dosage regimen provides a continuous infusion or a series of doses that reduce the fluctuation between the peak and trough plasma concentrations of the active ingredient. In some embodiments, a programmed dosage regimen provides a clinically effective steady state plasma concentration of an active ingredient such as ketamine with a peak trough fluctuation of no more than 5%, 10%, 20%, 30%, 40%, 50%, 60% 70%, 80%, 90%, 100%, 150%, 200%, 250%, 300%, 350%, 400%, 450%, 500%, 600%, 700%, 800%, 900%, or 1000% or more of the average steady state concentration during treatment. In some embodiments, a programmed dosage regimen provides a clinically effective steady state plasma concentration of an active ingredient such as ketamine with a peak to trough ratio of no more than 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 or more.

In some embodiments, a programmed dosage regimen provides an effective steady state drug plasma concentration (e.g., ketamine) while providing relatively low peak trough fluctuation. In some embodiments, a programmed dosage regimen provides a steady state drug plasma concentration of at least 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL and/or a peak to trough ratio of no more than 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 or more.

In some embodiments, a programmed dosage regimen provides a steady state drug plasma concentration (e.g., ketamine) having a Cₘₐₓ of no more than 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL, and/or a Cₘᵢₙ of at least 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL.

In some embodiments, a programmed dosage regimen provides an effective steady state drug plasma concentration (e.g., ketamine) having a Cₘₐₓ to Cₘᵢₙ ratio of no more than 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10.0, 11, 12, 13, 14, 15, 16, 17, 18, 19, 20, 25, 30, 35, 40, 45, or 50 or more.

### Pharmaceutical Compositions

The terms “pharmaceutical composition” and “drug formulation,” as used herein, are synonymous.

In an aspect, provided herein is a pharmaceutical composition, comprising:
(i) aa drug molecule, or a hydrate, solvate, or pharmaceutically acceptable salt thereof; and
(ii) at least one pharmaceutically acceptable excipient,
wherein the pharmaceutical composition is in a form for dosing or administration by intravenous (I.V.), intramuscular, subcutaneous, or intradermal injection.

In some embodiments, the drug formulation or pharmaceutical composition administered according to the systems, devices, kits, formulations, and methods disclosed herein is a liquid formulation such as an aqueous solution. In some embodiments, the formulation or pharmaceutical composition is configured to be administered by intramuscular injection. In some embodiments, the formulation or pharmaceutical composition is configured to be administered by subcutaneous injection. In some embodiments, the formulation or pharmaceutical composition is configured to be administered by intravenous injection. In some embodiments, the formulation or pharmaceutical composition is administered continuously as an infusion. In some embodiments, the formulation or pharmaceutical composition is administered by injection as a bolus. In some embodiments, the formulation or pharmaceutical composition is administered by injection as a bolus over a period of time such as about 10 minutes.

In some embodiments, the formulation is configured to be administered through a pump device, as described herein.

In certain embodiments of the pharmaceutical compositions described herein, the at least one pharmaceutically acceptable excipient is (i) a surface-active agent, (ii) a non-ionic surfactant, (iii) a phospholipid solubilization agent, (iv) a cyclodextrin excipient, (v) an emulsion stabilizer, (vi) a preservative, (vii) an antimicrobial agent, or (viii) a topical analgesic. In some embodiments, the topical analgesic is lidocaine.

In certain embodiments of the pharmaceutical compositions described herein, the dosage form is an I.V. dosage form.

In some embodiments, the formulation or pharmaceutical composition comprises an active ingredient at a concentration of at least about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, or 500 mg/mL or more and/or no more than about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, or 500 mg/mL or more. In some embodiments, the formulation comprises an active ingredient such as ketamine at a concentration of about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, or 500 mg/mL or more. In some embodiments, the formulation comprises an active ingredient such as ketamine at a concentration of about 10 mg/mL to about 300 mg/mL.

In some embodiments, the formulation or pharmaceutical composition is a pharmaceutical composition. In some embodiments, the formulation is in the form of a sterile injectable aqueous or oleaginous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, NJ) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin). In some embodiments, the formulation comprises a co-solvent. In some embodiments, a suitable co-solvent is propylene glycol, glycerin, ethanol, polyethylene glycol (300 and 400), Sorbitol, dimethylacetamide, Cremophor EL, or N-methyl-2-pyrrolidone, or dimethylsulfoxide.

In some embodiments, the formulation or pharmaceutical composition is an aqueous suspension. Aqueous suspensions contain active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives (e.g., benzethonium chloride).

In some embodiments, the formulation or pharmaceutical composition comprises a stabilization agent. In some embodiments, the formulation comprises a surface-active solubilization agent. Surface-active solubilization agents include, but are not limited to: polyoxyethylene sorbitan monooleate (Tween 80), sorbitan monooleate, polyoxyethylene sorbitan monolaurate (Tween 20), lecithin, and Polyoxyethylene-polyoxypropylene copolymers (Pluronics1). In some embodiments, the formulation comprises a non-ionic surfactant solubilization agent. Non-ionic surfactants include, but are not limited: Cremophor RH 40, Cremophor RH 60, d-alpha-tocopherol polyethylene glycol 1000 succinate, polysorbate 20, polysorbate 80, Solutol HS 1, sorbitan monooleate, poloxamer 407, Labrafil M-1944CS, Labrafil M-2125CS, Labrasol, Gellucire 44/14, Softigen 767, and mono-fatty esters and di-fatty acid esters of PEG 300, 400, and 1750. In some embodiments, the formulation comprises a phospholipid solubilizing agent such as, hydrogenated soy phosphatidylcholine, phosphatidylcholine, distearoylphosphatidylglycerol, L-alpha-dimyristoylphosphatidylcholine, or L-alpha-dimyristoylphosphatidylglycerol.

In some embodiments, the formulation or pharmaceutical composition comprises a complexation agent. In some embodiments, the complexation agent is hydroxypropyl-b-cyclodextrin, bulfobutylether-b-cyclodextrin (Captisol1), or polyvinylpyrrolidone. In some embodiments, the complexation agent is an amino acid such as, arginine, lysine, or histidine.

The formulations or pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The formulation or pharmaceutical composition typically comprises a therapeutically effective amount of an active compound, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a triethanolamine (Tris) buffer, histidine, bicarbonate; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

Many active pharmaceutical ingredients (APIs) are weak acids or weak bases. Weak acids or weak bases can exist in an un-ionized form or as an ionized complex prepared by the addition of a base or acid respectively. The resultant complex is stabilized by ionic interactions and is known as a salt. This complex exists via an ionic bond between an ionized API and an oppositely charged counterion. Salts offer a number of advantages over their un-ionized counterparts. The choice of counterion can have a large influence on the salts properties and the use of a given salt form of a given API in a pharmaceutical product is influenced and guided by a number of factors for example stability (photo, hydrolytic and thermal), solubility, physicochemical properties, solid state properties (crystallinity, polymorphism, particle size, crystal morphology, melting point, compactability), production considerations (e.g., ease of handling and processing), dissolution rate, modulation of drug release, compatibility with excipients and containers, ease and consistency of production, desired route of administration, and organoleptic factors (e.g., taste). Furthermore, with respect to injection, salt can influence pain and irritation at the injection site (Brazeau et al. 1998).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations or pharmaceutical compositions can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. The drug delivery devices described herein may be used to deliver the formulations.

In some embodiments, the formulation or pharmaceutical composition is stored in a reservoir of the drug delivery device. In some embodiments, the formulation is stored in a cartridge that is insertable and/or attachable to the drug delivery device. In some embodiments, the cartridge and/or drug delivery device comprises a product label for intramuscular injection. In some embodiments, the cartridge and/or drug delivery device comprises a product label for subcutaneous injection. In some embodiments, the cartridge and/or drug delivery device comprises a product label for intravenous injection. In some embodiments, disclosed herein is a kit comprising a product label for intramuscular injection. In some embodiments, disclosed herein is a kit comprising a product label for subcutaneous injection. In some embodiments, disclosed herein is a kit comprising a product label for intravenous injection.

In general, dosing parameters dictate that the dosage amount be less than an amount that could be irreversibly toxic to the subject (the maximum tolerated dose (MTD) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of the drug of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the dosage of the drug is contained in a “unit dosage form.” The phrase “unit dosage form” refers to physically discrete units, each unit including a predetermined amount of the compound (e.g., ketamine, or a hydrate, solvate, or pharmaceutically acceptable salt thereof), sufficient to produce the desired effect. It will be appreciated that the parameters of a unit dosage form will depend on the particular agent and the effect to be achieved.

In some embodiments, the formulation or pharmaceutical composition is a liquid formulation comprising ketamine. In some embodiments, the formulation comprises a racemic ketamine composition. Alternatively, in some embodiments, the formulation comprises a substantially pure stereoisomer of ketamine (e.g., over 90%, 95%, 96%, 97%, 98%, or 99% of the ketamine is one stereoisomer). In some embodiments, the formulation comprises substantially pure S-ketamine. In some embodiments, the formulation comprises substantially pure R-ketamine. In some embodiments, the ketamine is at least about 90%, about 95%, about 96%, about 97%, about 98%, or about 99% pure. In some embodiments, the NMDA receptor antagonist is at least about 99.1%, about 99.2%, about 99.3%, about 99.4%, about 99.5%, about 99.6%, about 99.8%, or about 99.9% pure. In some embodiments, the NMRA receptor antagonist comprises less than about 5%, about 4%, about 3%, about 2%, or about 1% impurities.

In some embodiments, the formulation or pharmaceutical composition is a liquid formulation comprising a Schedule 1 drug. In some embodiments, the formulation or pharmaceutical composition is a liquid formulation comprising a Schedule 2 drug. In some embodiments, the formulation or pharmaceutical composition is a liquid formulation comprising a Schedule 3 drug. In some embodiments, the formulation or pharmaceutical composition is a liquid formulation comprising a Schedule 4 drug. In some embodiments, the formulation or pharmaceutical composition is a liquid formulation comprising an opioid drug. In some embodiments, the formulation or pharmaceutical composition is a liquid formulation comprising a drug with a potential for abuse. In some embodiments, the formulation or pharmaceutical composition is a liquid formulation comprising a drug with a potential for addiction. In some embodiments, the formulation or pharmaceutical composition is a liquid formulation comprising a high priced drug.

### Tamper Resistant Devices and Cartridges

Disclosed herein are systems, devices, and methods that provide tamper resistant features to prevent or reduce the risk of unauthorized use or abuse. In some embodiments, the tamper resistant features comprise safety features to prevent injury or harm. In some embodiments, tamper resistant features include physical or mechanical elements or properties designed to resist tampering such as attempts to penetrate the drug delivery device, drug reservoir, or drug cartridge (e.g., reinforced walls or surface). The drug reservoir can be in a prefilled primary container such as, for example, a syringe, standard cartridge, flexible bag, bellows, or custom cartridge.

In some embodiments, a tamper resistant feature comprises a sliding lock-off window that permanently secures the filling port on an internally integrated reservoir from any further access after it is filled by a pharmacist, or doctor, or a certified service, or a manufacturer.

In some embodiments, a tamper resistant feature a sliding lock-off window that secures the filling port on an internally integrated reservoir after it is filled by a pharmacist, or doctor, or a certified service, or a manufacturer in a fashion that is reversible with a physical key, or an electronic key, password or other biometric identification system.

In some embodiments, a tamper resistant feature comprises an internal or external locking system that secures a disposable drug reservoir from any further access after it is inserted by a pharmacist, or doctor, or a certified service or a manufacturer.

In some embodiments, a tamper resistant feature comprises an internal or external locking system that secures a disposable drug reservoir after it is inserted by a pharmacist, or doctor, or a certified service or a manufacturer in a fashion that is reversible with a physical key, or an electronic key, password or other biometric identification system programmed into the device.

In some embodiments, a tamper resistant feature comprises a self-contained motion detection system (e.g., accelerometer) or GPS related motion detection system. In some embodiments, the motion detection system is configured to monitor one or more biometric parameters such as movement, velocity and/or acceleration during certain treatment modes (e.g., bolus dosing) in order to detect non-sanctioned behavior (e.g., driving, walking, running). In some embodiments, detection of non-sanctioned behavior signals a potential need for modification of treatment parameters either automatically (e.g., as per firmware programming) or as per the discretion and/or direction of a remote treating physician or other certified person. In some embodiments, the modification comprises shutting down the device, locking off any further use without oversight, notifying the treating physician of potential non-sanctioned use, changing the delivery parameters remotely, or any combination thereof. In some embodiments, the systems, devices, and methods disclosed herein are configured to modify the treatment parameters upon detection of non-sanctioned behavior. In some embodiments, the modification occurs after a threshold number of incidents of non-sanctioned behavior have been detected. In some embodiments, the modification occurs after at least 1, 2, 3, 4, 5, 6, 7, 8, 9, or 10 or more incidents of non-sanctioned behavior have been detected.

In some embodiments, tamper resistant features provide inactivation and/or neutralization of the active ingredient of the drug formulation stored in the device and/or cartridge upon detection of a breach or tampering attempt. In some embodiments, a breach is detected based on a pressure change. In some embodiments, a breach triggers the release of one or more components configured to prevent unauthorized use of the liquid drug formulation. In some embodiments, a breach triggers the release of activated charcoal into the liquid drug formulation to absorb the active ingredient. In some embodiments, a breach triggers the release of a biocompatible gel forming polymer to convert the liquid drug formulation into a gel or solid (e.g., so as to reduce or prevent injection of the drug formulation). In some embodiments, a gel forming polymer is gellan gum, alginic acid, xyloglucan, pectin, chitosan, poly(DL-lactic acid), poly(DL-lactide-co-glycolide), or poly-caprolactone. In some embodiments, the drug delivery device and/or drug cartridge comprises a filter disposed between the liquid drug formulation and the injection site to prevent injection of one or more components solids or particles into the subject. For example, accidental damage to the drug delivery device or cartridge may cause activated charcoal to be released into the liquid drug formulation, but the presence of the filter prevents any of the charcoal from being injected into the patient.

In some embodiments, the drug delivery device and/or drug cartridge comprises a filter for filtering the liquid drug formulation. In some embodiments, the filter is a 0.1 micron filter. In some embodiments, the filter comprises a cellulose nitrate, cellulose acetate, nylon, polyether-sulfone, regenerate cellulose, or PTFE membrane. In some embodiments, the filter has a pore size of at least 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or at least 5.0 microns or more and/or a pore size of no more than 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or at least 5.0 microns or more. In some embodiments, the filter has a pore size of about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or at least 5.0 microns or more. In some embodiments, the filter is a 0.8 micron filter. In some embodiments, the filter is a 0.45 micron filter. In some embodiments, the filter is a 0.2 micron filter. In some embodiments, the filter is a 0.22 micron filter.

In some embodiments, tamper resistant features include software restrictions on access to the dosage regimen or dosing parameters. For example, in some embodiments, a software restriction is a password authentication requirement for a user to configure or modify a dosage regimen or an individual dose. In some embodiments, a software restriction is a biometric authentication step required for a user to configure or modify a dosage regimen or an individual dose (e.g., via a fingerprint scanner on the drug delivery device). In some embodiments, a drug delivery device comprises at least one processor and instructions executable by the at least one processor to create an application comprising a software module carrying out an authentication step. In some embodiments, a drug delivery device comprises an authentication module for authenticating a user or authorized user. In some embodiments, an authentication module provides at least two levels of access. In some embodiments, an authentication module grants access for a user or subject to administer a dose according to a dosage regimen, but restricts or limits the ability to configure or modify the dosage regimen. In some embodiments, an authentication module grants access to an authorized user to configure or modify the dosage regimen. As an example, an authentication module grants a patient's doctor the ability to configure a dosage regimen upon entry of an authentication code, and subsequent grants the patient the ability to administer a dose based on biometric identification using the patient's fingerprint.

In some embodiments, the drug delivery device monitors delivery of the drug formulation for each cartridge. In some embodiments, the drug delivery device logs each administration of the drug formulation for each cartridge. For example, in some embodiments, logged information includes at least one of cartridge ID (e.g., lot number, serial number, an arbitrary assigned number or ID, or some other identifying information), remaining volume, concentration, time and/or date of infusion, duration of infusion, infusion rate, and administered dose (e.g., volume). In some embodiments, the drug delivery device communicates the logged information to a remote authorized user (e.g., via a server or communication device accessible by the authorized user). In some embodiments, a cartridge provides identifying information detectable by the drug delivery device. In some embodiments, a cartridge provides identifying information via an RFID (radio frequency identification), microchip, barcode, magnetic stripes, or other mechanism for providing identifying information. In some embodiments, a drug delivery device comprises a detector or reader for obtaining identifying information from the cartridge.

In some embodiments, tamper resistant features include tamper evident packaging that indicates unauthorized use or access to the stored drug formulation. For example, in some embodiments, a subject must return or present one or more disposable cartridges when seeking to obtain more cartridges (e.g., refilling or renewing a prescription) at which point a healthcare provider can examine the device and/or cartridge for signs of tampering (e.g., damage or breach). In some embodiments, the prescription refill or renewal is denied when tampering is detected. In some embodiments, the doctor or healthcare provider who gave the prescription is informed of the tampering.

In some embodiments, a drug delivery device monitors attempts to configure or modify the dosage regimen. In some embodiments, the drug delivery device maintains a log of attempts to configure or modify the dosage regimen. In some embodiments, the drug delivery device maintains a log of all changes to the dosage regimen. In some embodiments, the drug delivery device communicates one or more attempts to configure/modify the dosage regimen and/or one or more changes to the dosage regimen over a network to a remote authorized user (e.g., the subject's doctor). In some embodiments, communications to the remote authorized user are stored on a server or network device that is accessible by the remote authorized user (e.g., viewable over the Internet via a web API).

In some embodiments, tamper resistant features include preloaded cartridges to avoid the need for subjects to self-charge the devise with the formulation. In some embodiments, tamper resistant features a rubber membrane of sufficient thickness on preloaded cartridges to prohibit access to the formulation by means other than the access port needle on the accompanying catheter. In some embodiments, tamper resistant features include lockout times to be determined by a user during which the subject cannot select and administer a treatment. In some embodiments, a lockout time is initiated upon detection of an attempt to tamper with the device and/or administer one or more doses outside of the subject's authorized use. For example, repeated attempts to increase the dosage beyond a preset dosage limit may initiate a lockout time. In some embodiments, a lockout time is a period during which device access is locked such that a dose cannot be administered by the subject. In some embodiments, the device is locked out during an ongoing dose (e.g., user is self-administering a continuous infusion dose and repeatedly attempts to increase the dose beyond a dosage limit).

### Digital Processing Device

In some embodiments, the platforms, media, methods and applications described herein include a digital processing device 101, a processor 105, or use of the same. In further embodiments, the digital processing device 101 includes one or more hardware central processing units (CPU) 105 that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage 115 and/or memory 110 device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the non-volatile memory comprises magnetoresistive random-access memory (MRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a subject. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In some embodiments, the display is E-paper or E ink. In other embodiments, the display is a video projector. In still further embodiments, the display is a combination of devices such as those disclosed herein.

In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. In addition, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, subject, or network information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### Web Browser Plug-In

In some embodiments, the computer program includes a web browser plug-in. In computing, a plug-in is one or more software components that add specific functionality to a larger software application. Makers of software applications support plug-ins to enable third-party developers to create abilities that extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### EXAMPLES

### Example 1—Single Use Fully Integrated Device

An exemplary embodiment of a drug delivery device provided herein is shown in FIG. 2. The drug delivery device is a single use wearable patch pump with all aspects of the device configured as a single disposable component. The device has an adhesive patch for application to the skin of a subject such that the subject can wear the device for the duration of the dosing schedule. Positioned on the adhesive patch opposite the adhesive surface is a compartment comprising the remaining components of the device, including a drug formulation containing reservoir, a pumping mechanism configured to pump the drug, and a needle configured to deliver the drug formulation subcutaneously, a user interface, and electronic components. The user interface is positioned atop the drug delivery device and is visible from the exterior. The remaining components are positioned on the interior of the device, which is configured to be tamper resistant to prevent access by the subject to the drug formulation. In this embodiment, the user interface includes a button to activate the device, a pump status indicator light, and a light bar showing the amount of drug formulation remaining (e.g., bolus indicator), which can also act as a leveling indicator.

The button to activate the device is configured to start a dosing regimen of the drug formulation according to a pre-programmed protocol which is not alterable by the subject. Once configured to start the dosing regimen, the device is configured to deliver a controlled, titratable amount of the drug formulation to the subject over a specified period of time, which is a period over several days, weeks, or months. The drug formulation is a ketamine formulation for the treatment of pain with occasional bolus additions once every several hours may be allowed according to the pre-programmed protocol.

The user interface in this embodiment includes a pump status indicator which is a multi-colored light system programmed to display a different color depending on the status of the pump. The color of the pump status indicator is red when the pump is not operational, green when the pump is operational, and yellow when the pump is delivering a bolus addition, if allowed by the protocol.

### Example 2—Two Part Device Comprising a Reusable Component and a Disposable Component and Kit

An example of a two part device having a reusable component and a disposable component is shown in FIG. 3A, which illustrates a device having a reusable user interface component comprising the electronic components of the device, drive gearmotor, and power systems and a disposable component comprising a reservoir comprising a drug formulation, fluid path, and necessary drive components. The two distinct components are configured to be assembled by the subject in an easy to operate manner, such as by a simple clip mechanism. Only one disposable component can be assembled with the reusable user interface at a time. Once assembled, the device can have any of the features described with respect to the one part device, for example, as described in Example 1 with one or more of the following additional features. The drive components within the disposable component are driven by the power systems and gearmotor embodied within the reusable component and transmitted via the drive coupling interface as shown in FIGS. 3A-3E.

In this example, the transmission coupling between the reusable component and disposable component are magnetic utilizing one or more magnets on the reusable component. A benefit of the magnetic coupling interface is that there are no external features visible on the disposable component enclosure or the reusable component enclosure that indicate the drive coupling interface. This provides for a clean look on the external encloser surfaces and ease of waterproofing the reusable and disposable components.

The disposable component contains a complete drive system including an electric gearmotor that provides the transmission through a drivetrain that displaces the cartridge plunger providing fluid delivery through a patient administered fluid path to the patient. The gear motor is located within the disposable component, and electrical contacts between the reusable component to the disposable component are used to provide electrical power and drive control to the gear motor.

The reusable user interface portion is configured such that it will only operate with a specified disposable component or a specified plurality of disposable components according to a pre-determined treatment protocol. The disposable component is designed to be tamper resistant and configured not to deliver the drug formulation in the absence of the user interface portion configured to operate with it.

The disposable component in this example comprises a radio-frequency-identification (RFID) tag which can be read by an RFID reader positioned on the reusable user interface component. The RFID tag also contains information on the drug concentration and intended delivery parameters such as basal or bolus programming rates, amounts, and use duration.

The device provided in this example is prescribed to a subject by a medical professional as a kit, wherein the kit contains a single re-usable user interface and multiple disposable components containing the prescribed drug formulation (e.g., 5-10 for a long dose regimen). The kit so configured minimizes the risk of a subject administering more of the prescribed drug formulation than prescribed because the subject is prescribed only a single delivery device. By contrast, if a subject is prescribed multiple doses of the prescribed drug formulation via integrated devices without a removal cartridge, there is a risk the subject can place multiple devices on their body simultaneously in order to exceed the prescribed dose of the drug. This is a special concern for controlled substance drug formulation that are prone to abuse, such as ketamine. Additionally, the tamper proof nature of the individual disposable components also prevents the foreseeable misuse of drug. Thus, the device provided in this example provides distinct advantages to those of fully integrated single use devices.

### Example 3—Three-Part Device Comprising Two Disposable Components and a Single Reusable Component and Kit

An example of a three part delivery device is provided herein which has two distinct disposable components and a single reusable component. The three distinct components are configured to be assembled by a subject in an easy to operate manner, such as by simple clipping mechanisms. Instead of having a single disposable component comprising a reservoir comprising a drug formulation, fluid path, and necessary drive components as in the two part delivery device, the reservoir comprising the drug formulation is separate from the fluid path and drive components until assembled by the subject.

The first disposable component has a drug reservoir containing the drug formulation with the interior of the reservoir containing the drug formulation being sterilized at the time of manufacturing using fill and finish techniques. The drug formulation is sealed in the reservoir with a cartridge septum which can be readily pierced using a needle from the second disposable component.

The second disposable component has the fluid path and drive components to operate the device, as well as the adhesive surface configured to attach the device to the subject. This second disposable component also contains a needle configured to pierce the septum of the drug reservoir, thereby creating a fluid connection between the flow path and the drug formulation. The second disposable component is assembled by the manufacturer and packaged in a blister package, which is then sterilized by ethylene oxide gas.

The second disposable component is configured such that once the first disposable component is placed within the second disposable component, it is locked or latched in place and not removable. This ensures that when the two disposable components are coupled, access to the reservoir or cartridge septum and therefore medication container within is limited, providing additional abuse or misuse protection.

The device is provided as a kit including the reusable user interface component, a blister pack containing multiple second components comprising the fluid path and adhesive surface for attaching the device to the subject, and a tamper resistant (TR) package which contains the drug formulation filled reservoir components.

### Example 4—Priming and Air Removal Systems

This example describes a non-limiting embodiment of a priming and air removal system compatible with any suitable drug delivery device, including the single component, dual-component, and triple component drug delivery devices provided herein. For sustained, titratable delivery of drug formulations by subcutaneous or intramuscular injection, it is vitally important that an accurate amount of the drug be delivered according to the prescribed protocol. The presence of air in a sustained delivery system, such as the drug devices provided herein, could lead to an incorrect accounting and administration of the amount of drug actually delivered, thereby undermining the therapeutic potential of the intervention. The priming and air removal system provided in this example addresses this problem by providing a time-of-use priming of a wearable drug delivery device.

FIG. 6 shows a device containing a reservoir pre-filled with the drug formulation can contain air 604 as a result of the manufacturing process used to fill the reservoir. Pre-filled devices with reservoirs positioned on the interior of a device pose a special problem. This is because in order to expel trapped air, the device must be oriented such that the exit port of the reservoir is positioned up, thereby allowing the device to drive the air out of the reservoir without wasting the drug formulation disposed therein, as shown in FIG. 7. The plunger can be used to release any trapped air through the needle exit port. To ensure the device is in the proper orientation prior to priming the system to expel trapped air from the reservoir, the user interface in this embodiment comprises a light bar functioning as a levelling indicator. The light bar is configured such that each light can be red, green, or yellow. During initiation of the device and before it is applied to the subject, the device is primed to remove any air that is trapped in the drug formulation reservoir during manufacturing. To ensure the device is properly oriented, the light bar is configured to act as a levelling indicator for this priming step. During this priming step, the light bar is configured to display orientation information about the reservoir within the device to guide the user in priming the device to remove trapped air.

The accelerometer of this smart sensor is configured to monitor for excessive movements during transportation or manufacturing of the device which may have caused any trapped air to break up into smaller bubbles, and displays a red light in the event of the acceleration or movement exceeding a threshold prior to administration. Once both the pump status light and orientation lights are green, indicating the system is prepared for priming, priming of the system begins automatically to purge the trapped air.

### Example 5—Drive System Lockout

A two or three part/component device includes a drive system lockout mechanism comprising a secondary drive wheel latch system is incorporated between the reusable component and the durable component, as shown in FIGS. 10A-10B. An electromagnet, driven by the electronic board and control systems within the reusable component, is used to activate the drive wheel latch within the disposable component. FIGS. 10A-10B show the two-part pump in the locked state in the absence of the magnetic force provided by the electromagnet upon the ferrous metal plate 1026, in which the drive wheel latch 1024 is in the normal latched state, not allowing the drive wheel 1012 to rotate by keeping the drive locked 1022, thereby inhibiting unintended rotation of the drive interface through the drive coupling nut. The drive coupling interface is locked by the drive wheel latch until the electromagnet is activated to release the drive wheel.

### Example 6—Single Part Prefilled and Preloaded (Fixed Bolus Dose) Pen Injector

This example describes operation of a non-limiting embodiment of a prefilled, preloaded pen injector with a cap, activation button, and readiness window. The pen injector is operated as follows:
1. Remove the prefilled preloaded Pen Injector from the packaging
2. [Readiness Window on Pen Injector will show green, ready for delivery]
3. Remove Pen Injector Cap
4. Wipe the pen injector septum and attach a clean sterile needle
5. Use sterile wipe to clean injection location
6. Inject Dose
7. [Cap lockout extends, and Activation Button lockout feature is engaged. Timer starts within pen injector for lockout period]
8. Remove needle and dispose per local regulations
9. Re-attach Pen Cap
10. Monitor Readiness Window on Pen Injector. Will show 1) Yellow=in lockout, 2) Green=ready for next injection, 3) red=no more injections available]
11. When the Pen Readiness Window displays Green, device is ready for another injection, return to step 3 above
12. When the Pen Readiness Window displays Red, dispose of the pen injector following local regulations

### Example 7—Mobile Authorization—Single Part Prefilled and Preloaded (Fixed Bolus Dose) Pen Injector

This example describes operation of a non-limiting embodiment of a single part prefilled, preloaded pen injector with a cap, activation button, and readiness window configured for authorization for injection via a mobile device. The pen injector is operated as follows:
1. Download and Open the Injector App to your mobile device. Register through app to receive Pen Injector Unlock authorization
2. Remove prefilled preloaded Pen Injector from packaging
3. Press and hold the Activation Button on Pen Injector for 5 seconds to pair with mobile device. Pen LED will flash blue.
4. [Mobile device will send unlock signal to Pen Injector to unlock the Cap and Activation Button to allow injections at the pre-set controlled intervals, not changeable by the patient]
5. Pen LED will turn to green indicating ready for injection
6. Remove Pen Injector Cap
7. Wipe the pen injector septum and attach a clean sterile needle
8. [Mobile app will indicate that pen is ready for delivery, or lockout time remaining until next ready for delivery]
9. Use sterile wipe to clean injection location
10. Inject Dose
11. [Pen LED displays readiness color selected from the following states: 1) green=ready for next injection, 2) yellow=in lockout, 3) red indicates no more injections available, 4) flashing red means device error. For states 2-4, Cap lockout extends, and Activation Button lockout feature is engaged. Timer starts within pen injector for lockout period]
12. Remove needle and dispose per local regulations
13. Re-attach Pen Cap
14. When Pen LED turns green, go to step 6 above.
15. When the Pen LED turns to Red, dispose of the pen injector following local regulations

### Example 8—Mobile Authorization—Two Part Prefilled and Preloaded (Fixed Bolus Dose) Pen Injector

This example describes operation of a non-limiting embodiment of a two part prefilled, preloaded pen injector with a cap, activation button, and readiness window configured for authorization for injection via a mobile device. The pen injector is operated as follows:
1. Download and Open the Injector App to your mobile device. Register through app to receive Pen Injector Unlock authorization.
2. Remove Prefilled preloaded Pen Injector Body and the Reusable Pen Injector Driver from packaging
3. Attach the Pen Injector Body to the Reusable Pen Injector Driver
4. Press and hold the Activation Button on Pen Injector for 5 seconds to pair with mobile device. Pen LED will flash blue.
5. [Mobile device will send unlock signal to Pen Injector to unlock the Cap and Activation Button to allow injections at the pre-set controlled intervals, not changeable by the patient]
6. Pen LED will turn to green indicating ready for injection
7. Remove Pen Injector Cap
8. Wipe the pen injector septum and attach a clean sterile needle
9. [Mobile app will indicate that pen is ready for delivery, or lockout time remaining until next ready for delivery]
10. Use sterile wipe to clean injection location
11. Inject Dose
12. [Pen LED displays readiness color; 1) green=ready for next injection, 2) yellow=in lockout, 3) red indicates no more injections available, 4) flashing red means device error. For states 2-4, Cap lockout extends, and Activation Button lockout feature is engaged. Timer starts within pen injector for lockout period]
13. Remove needle and dispose per local regulations
14. Re-attach Pen Cap
15. When Pen LED turns green, go to step 6 above.
16. When the Pen LED turns to Red, remove the Pen Injector Body from the Pen Injector Driver and dispose of the Pen Injector Body following local regulations
17. If there are more filled Pen Injector Bodies available per the prescription, return to step 3.

### Example 9—Mobile Authorization—Single Part Prefilled and Preloaded Patient Adjustable (Dial-a-Dose Bolus Dose) Pen Injector

This example describes operation of a non-limiting embodiment of a single part prefilled, preloaded pen injector with a cap, activation button, dose set window, and readiness LED configured for authorization for injection via a mobile device. The pen injector is operated as follows:
1. Download and Open the Injector App to your mobile device. Register through app to receive Pen Injector Unlock authorization
2. Remove prefilled preloaded Pen Injector from packaging
3. Press and hold the Activation Button on Pen Injector for 5 seconds to pair with mobile device. Pen LED will flash blue.
4. [Mobile device will send unlock signal to Pen Injector to unlock the Cap and Activation Button to allow injections at the pre-set controlled intervals, not changeable by the patient]
5. Pen LED will turn to green indicating ready for injection
6. Patient will dial the intended delivery dose and verify the correct amount in the Dose Window
7. Remove Pen Injector Cap
8. Wipe the pen injector septum and attach a clean sterile needle
9. [Mobile app will indicate that pen is ready for delivery, or lockout time remaining until next ready for delivery]
10. Use sterile wipe to clean injection location
11. Inject Dose
12. [Pen LED displays readiness color selected from the following states: 1) green=ready for next injection, 2) yellow=in lockout, 3) red indicates no more injections available, 4) flashing red means device error. For states 2-4, Cap lockout extends, and Activation Button lockout feature is engaged. Timer starts within pen injector for lockout period]
13. Remove needle and dispose per local regulations
14. Re-attach Pen Cap
15. When Pen LED turns green, go to step 6 above.
16. When the Pen LED turns to Red, dispose of the pen injector following local regulations.

### Example 10—Mobile Authorization—Two Part Prefilled and Preloaded Patient Adjustable (Dial-a-Dose Bolus Dose) Pen Injector

This example describes operation of a non-limiting embodiment of a two part prefilled, preloaded pen injector with a cap, activation button, dose set window, and readiness LED configured for authorization for injection via a mobile device. The pen injector is operated as follows:
1. Download and Open the Injector App to your mobile device. Register through app to receive Pen Injector Unlock authorization.
2. Remove Prefilled preloaded Pen Injector Body and the Reusable Pen Injector Driver from packaging
3. Attach the Pen Injector Body to the Reusable Pen Injector Driver
4. Press and hold the Activation Button on Pen Injector for 5 seconds to pair with mobile device. Pen LED will flash blue.
5. [Mobile device will send unlock signal to Pen Injector to unlock the Cap and Activation Button to allow injections at the pre-set controlled intervals, not changeable by the patient]
6. Pen LED will turn to green indicating ready for injection
7. Patient will dial the intended delivery dose and verify the correct amount in the Dose Window
8. Remove Pen Injector Cap
9. Wipe the pen injector septum and attach a clean sterile needle
10. [Mobile app will indicate that pen is ready for delivery, or lockout time remaining until next ready for delivery]
11. Use sterile wipe to clean injection location
12. Inject Dose
13. [Pen LED displays readiness color; 1) green=ready for next injection, 2) yellow=in lockout, 3) red indicates no more injections available, 4) flashing red means device error. For states 2-4, Cap lockout extends, and Activation Button lockout feature is engaged. Timer starts within pen injector for lockout period]
14. Remove needle and dispose per local regulations
15. Re-attach Pen Cap
16. When Pen LED turns green, go to step 6 above.
17. When the Pen LED turns to Red, remove the Pen Injector Body from the Pen Injector Driver and dispose of the Pen Injector Body following local regulations
18. If there are more filled Pen Injector Bodies available per the prescription, return to step 3.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A drug delivery system comprising a delivery device comprising:
a) a pump or injection mechanism configured for administering a drug formulation from a reservoir through a delivery needle; and
b) an activation mechanism configured to selectively lock the pump or injection mechanism to prevent administration of the drug formulation.

2. The drug delivery system of claim 1, wherein the activation mechanism is configured to allow setting of a dosage of the drug formulation.

3. The drug delivery system of claim 1, wherein the delivery device is a single part prefilled injector, a two part prefilled injector, or a three part injector.

4. The drug delivery system of claim 1, wherein the delivery device comprises one or more of a reusable injector component comprising an electronic control module or a disposable component comprising the reservoir containing the drug formulation.

5. The drug delivery system of claim 1, wherein the delivery device comprises a magnetic or mechanical coupling mechanism for combining a reusable component and a disposable component making up the delivery device.

6. The drug delivery system of claim 1, wherein the delivery device comprises a shield activated trigger for unlocking the delivery device for administration of the drug formulation.

7. The drug delivery system of claim 1, further comprising a tamper resistant package comprising one or more cartridges containing the reservoir.

8. The drug delivery system of claim 7, wherein the drug delivery system is configured to cause the tamper resistant package to release a cartridge from the one or more cartridges upon obtaining user authorization, optionally wherein the plurality of cartridges are individually locked prior to obtaining user authorization.

9. The drug delivery system of claim 1, further comprising a cap assembly, optionally wherein the cap assembly comprises a decontamination sponge.

10. The drug delivery system of claim 1, further comprising a controlled cartridge septum lockout function, optionally wherein the controlled cartridge septum lockout function is configured to open or close an iris to control access to the reservoir.

11. A drug delivery system comprising:
a reservoir comprising a drug formulation;
a drive mechanism configured to pump the drug formulation from the reservoir through a delivery needle upon activation; and
a lockout mechanism configured to prevent unauthorized activation of the drive system.

12. The drug delivery system of claim 11, wherein the drug delivery system has a multi-component configuration comprising:
a reusable component; and
a disposable component housing the reservoir comprising the drug formulation.

13. The drug delivery system of claim 12, wherein the lockout mechanism keeps the drive mechanism locked until the reusable component and the disposable component are coupled.

14. The drug delivery system of claim 13, wherein the lockout mechanism comprises a magnet and a magnetic detection element that are brought into proximity upon coupling of the reusable component and the disposable component.

15. The drug delivery system of claim 14, wherein the magnet is an electromagnet housed within the reusable component and the magnetic detection element is housed within the disposable component, optionally wherein coupling of the reusable component and the disposable component causes an electromagnetic field produced by the electromagnet to act upon the magnetic detection element, thereby unlocking the drive mechanism for authorized activation to administer the drug formulation.

16. The drug delivery system of claim 13, wherein the lockout mechanism is configured to provide active electronic control or passive mechanical control over activation of the drive mechanism, optionally wherein the lockout mechanism configured to provide active electronic control unlocks the drive mechanism using RFID, Near Field Communication (NFC), or radio frequency.

17. The drug delivery system of claim 13, wherein the drug delivery system comprises an electronic module configured to provide active control over activation of the drive mechanism.

18. The drug delivery system of claim 17, wherein the electronic module is configured to control the drive mechanism based on receipt of a signal indicating authorized activation, optionally wherein the signal indicating authorized activation is provided by a wireless signal from a computing device.

19. The drug delivery system of claim 18, wherein the computing device is a desktop computer, a laptop computer, a tablet, or a smartphone.

20. The drug delivery system of claim 11, wherein the drug delivery system comprises a wearable pump.

21. The drug delivery system of claim 13, wherein the lockout mechanism is a completely mechanical mechanism providing control over activation of the drive mechanism without an electronic module.

22.-48. (canceled)
